(19) United States
(12) Patent Application Publication
(10) Pub. No.: US 2026/0347417 A1
(43) Pub. Date: Nov. 5, 2026

(54) NPY2 RECEPTOR AGONISTS

(71) Applicant: Boehringer Ingelheim International GmbH, Ingelheim am Rhein (DE)

(72) Inventors: Peter Wilhelm HAEBEL, Mittelbiberach (DE); Albert BRENNAUER, Riedlingen (DE); Charlotte Stahl MADSEN, Malov (DK); Soren Ljungberg PEDERSEN, Borup (DK); Stefan PETERS, Biberach an der Riss (DE)

(21) Appl. No.: 19/456,389

(22) Filed: Jan. 22, 2026

Related U.S. Application Data
(63) Continuation of application No. 18/751,723, filed on Jun. 24, 2024, now Pat. No. 12,559,529.
Application No. 18/751,723 is a division of application No. 18/068,641, filed on Dec. 20, 2022, now Pat. No. 12,054,525.
Application No. 18/068,641 is a continuation of application No. 17/093,680, filed on Nov. 10, 2020, now abandoned.

(30) Foreign Application Priority Data
Nov. 11, 2019 (EP) 19208394.7

(51) Int. Cl. C07K 14/47; A61K 38/00

(52) U.S. Cl. CPC C07K 14/47; A61K 38/00

(57) ABSTRACT

The invention relates to PYY analogues having alanine at position 4, lysine at position 7, QRY as the C-terminal end and a half-life extending group. The analogues of the invention are soluble around pH 6 and 7. The invention also relates to pharmaceutical compositions comprising such PYY analogues, and to the medical use of the analogues.

### RELATED APPLICATION DISCLOSURE

This application is a continuation of U.S. application Ser. No. 18/751,723, pending, which in turn is a divisional of U.S. application Ser. No. 18/068,641 filed Dec. 20, 2022, now U.S. Pat. No. 12,054,525, which claims the benefit of U.S. application Ser. No. 17/093,680 filed Nov. 10, 2020, abandoned, which claims the benefit of EP Provisional Application No. EP 19208394.7 filed Nov. 11, 2019, each of which is hereby incorporated by reference herein in its entirety.

### SEQUENCE LISTING

The instant application contains a Sequence Listing which has been submitted electronically in XML format and is hereby incorporated by reference in its entirety. Said XML copy, created on Apr. 14, 2023, is named 01-3375-US-2.xml and is 692,949 bytes in size.

### FIELD OF THE INVENTION

The present invention relates to PYY analogues that are neuropeptide Y2 (NPY2) receptor agonists, and to their medical use in the treatment and/or prevention of a variety of diseases, conditions or disorders, such as treatment and/or prevention of excess food intake, excess body weight, obesity, metabolic diseases, and other conditions or disorders related to excess body weight or obesity, e.g. diabetes and cardiovascular diseases.

### BACKGROUND OF THE INVENTION

Overweight and obesity are defined as abnormal or excessive fat accumulation that presents a risk to health. In this regard, overweight and obesity are major risk factors for a number of chronic diseases, including type 2 diabetes, cardiovascular diseases and cancer. According to the WHO overweight and obesity are no longer considered a problem limited to high income countries but are now dramatically on the rise in low- and middle-income countries. WHO's Global Health Observatory indicate that, in 2016, 39% of women or men aged 18 and over were overweight and 11% of men and 15% of women were obese.

Despite long-standing efforts, the number of overweight and obese patients is still growing. First line therapy for overweight and obese patients comprise diet and exercise but often are not sufficiently efficacious. Second line treatment options are bariatric surgery and pharmacotherapy. Available pharmacological treatments seem to lack in efficacy and/or safety, and only a limited number of approved therapies are available in the US and in Europe.

Therefore, there is still a high medical need for more efficacious and safe treatment options.

NPY (Neuropeptide Y; SEQ ID No:1—human sequence), PYY (Peptide YY; SEQ ID No: 2—human sequence), and PP (Pancreatic Polypeptide; SEQ ID No:3—human sequence) are naturally secreted homologous, 36 amino acid, C-terminally amidated peptides and belong to the PP-fold family of peptides.

Sequence of hPYY (3-36):

(SEQ ID No: 4)
Ile Lys Pro Glu Ala Pro Gly Glu Asp Ala Ser Pro
Glu Glu Leu Asn Arg Tyr Tyr Ala Ser Leu Arg His
Tyr Leu Asn Leu Val Thr Arg Gln Arg Tyr

PYY is cleaved to PYY(3-36) by dipeptidyl peptidase IV (DPP IV). PYY(3-36) displays increased selectivity for the neuropeptide Y2 receptor over neuropeptide Y1, Y4 and Y5 receptors as compared to PYY(1-36), albeit some Y1 and Y5 affinity is retained. It is believed that PYY or PYY(3-36) exhibits the feeding suppressive action via activation of the neuropeptide Y2 receptor (Inhibition of Food Intake in Obese Subjects by Peptide YY₃₋₃₆, N Engl J Med 2003; 349; 941-8).

However, PYY and also PYY(3-36) have a short half-life in the body and show undesirable chemical or physical properties, e.g. low stability. Further, the pharmacologic effect, e.g. its efficacy as body weight lowering agent, seems limited.

WO2014/178018 discloses PYY analogues and their ability to reduce food intake in mice. WO2011/033068 and WO2011/058165 disclose long acting Y2 receptor agonists. WO2015/071355, WO2016/198682 and WO2020/092191 relate to PYY compounds, which are selective Y2 receptor agonists. PYY compounds are disclosed comprising a covalently attached substituent or modifying group also referred therein as a protracting moiety.

There is a need in the art for further (long acting) PYY analogues selectively acting on the NPY2 receptor. For example, it would be desirable to increase further the solubility of PYY analogues, preferably to increase the solubility around pH 7 and/or around pH 6. This would increase the formulation options for a ready-to-use application and potentially allow combinations with other (peptide) therapeutics to improve their efficacy.

It has been found that the PYY analogues of the present invention generally are soluble around pH 6 and pH 7.

### BRIEF SUMMARY OF THE INVENTION

In a first aspect, the invention provides a PYY analogue, wherein the analogue comprises
i) alanine at the position corresponding to position 4 of hPYY(1-36)
ii) lysine at the position corresponding to position 7 of hPYY(1-36)
iii) the sequence QRY at its C-terminal end,
and wherein a half-life extending group is attached to the epsilon amino group of the lysine at position 7 or of a lysine at positions 6, 10, 11, 14, 17, 21, 22 or 30, or to the carboxylic acid group of the side chain of a aspartate or a glutamate at positions 14 or 30.

In some embodiments, the PYY analogue comprises
i) alanine at the position corresponding to position 4 of hPYY(1-36)
ii) lysine at the position corresponding to position 7 of hPYY(1-36)
iii) glutamate at the position corresponding to position 9 of hPYY(1-36)
iv) tyrosine at the position corresponding to position 20 of hPYY(1-36)
v) tryptophan at the position corresponding to position 30 of hPYY(1-36)
vi) leucine at the position corresponding to position 31 of hPYY(1-36)
vii) the sequence RQRY (SEQ ID NO: 258) at its C-terminal end,
and wherein a half-life extending group is attached to the epsilon amino group of the lysine at position 7 or of a lysine at positions 6, 10, 11, 14, 17, 21 or 22, or to the carboxylic acid group of the side chain of a aspartate or a glutamate at position 14.

In some embodiments, the PYY analogue comprises
i) alanine at the position corresponding to position 4 of hPYY(1-36)
ii) lysine at the position corresponding to position 7 of hPYY(1-36)
iii) glutamate at the position corresponding to position 9 of hPYY(1-36)
iv) tyrosine at the position corresponding to position 20 of hPYY(1-36)
v) the sequence WLTRQRY (SEQ ID NO: 259) at its C-terminal end,
and wherein a half-life extending group is attached to the epsilon amino group of the lysine at position 7 or of a lysine at positions 6, 10, 11, 14, 17, 21, or 22, or to the carboxylic acid group of the side chain of a aspartate or a glutamate at position 14.

In some embodiments, the PYY analogue is in accordance to one of the above embodiments and bears a half-life extending group, which is attached to the epsilon amino group of the lysine at position 7.

In some embodiments of the present invention, the PYY analogue is a compound having the formula:

R¹—Z—R²,
wherein R¹ is hydrogen, —C(O)C₁₋₆ alkyl, —C(O)C₆H₆, —C(O)C₃₋₆ cycloalkyl, —C(O)C₁₋₆ alkyl-C₃₋₆ cycloalkyl, or C₁₋₆ alkyl or C₁₋₆ alkyl-C₃₋₆ cycloalkyl:
R² is OH or NHR³, wherein R³ is hydrogen or C₁₋₃ alkyl; and
Z is an amino acid sequence of formula III (SEQ ID NO: 256):

Ala-Pro-X6-Lys-Pro-X9-X10-X11-X12-X13-X14-X15-X16-X17-X18-X19-Tyr-X21-Val-X23-Leu-Arg-His-Tyr-Tyr-Asn-Trp-Leu-Thr-Arg-Gln-Arg-Tyr (III)
wherein
X6 is selected from the group consisting of Ala and Glu;
X9 is selected from the group consisting of Glu and Gly;
X10 is selected from the group consisting of Ala and Glu;
X11 is selected from the group consisting of Ala, Asp, Glu and Pro;
X12 is selected from the group consisting of Ala and Ser;
X13 is selected from the group consisting of Ala, Glu, Ser, Thr and Pro;
X14 is selected from the group consisting of Ala, Glu and Pro;
X15 is selected from the group consisting of Ala and Glu,
X16 is selected from the group consisting of Ala and Glu;
X17 is selected from the group consisting of Ile, Leu, Thr and Val;
X18 is selected from the group consisting of Glu and Gln;
X19 is selected from the group consisting of Ala, Glu, Arg, Lys, and Gln;
X21 is selected from the group consisting of Glu and Tyr;
X23 is selected from the group consisting of Ala, Glu, Ser and Thr;
and wherein a half-life extending group is attached to the epsilon amino group of the lysine at position 7.

In some embodiments, the half-life extending group consists of a lipophilic substituent X and a linker U, wherein the linker U is attached to the amino acid side chain and X is attached to U, and the linker U consists of one, two or three sub-moieties (U1, U2, U3).

In some embodiments, the lipophilic substituent X is selected from the group consisting of 15-carboxy-pentadecanoyl, 17-carboxy-heptadecanoyl (C18DA) and 19-carboxy-nonadecanoyl, and the linker U consists of one, two or three sub-moieties independently selected from the group consisting of Gly, Glu, γ-Glu, ε-Lys, Ser, and OEG, or independently selected from the group consisting of γ-Glu, and OEG.

In some embodiment the PYY analogue is selected from the compounds 1 to 244 described herein.

In some embodiments the PYY analogue is in the form of a salt, preferably in the form of a pharmaceutically acceptable salt.

The invention further provides a composition comprising a PYY analogue as described herein.

The present invention further provides a PYY analogue for use in a method of medical treatment, e.g. for use in the treatment of obesity and various obesity-related conditions, diseases, or disorders such as type 2 diabetes, NAFLD or NASH.

The invention provides a PYY analogue of the invention for use in a method of treating, inhibiting or reducing weight gain, promoting weight loss and/or reducing excess body weight.

### DETAILED DESCRIPTION OF THE INVENTION

### Terms, Definitions and Conventions

Terms not specifically defined herein should be given the meanings that would be given to them by one of skill in the art in light of the disclosure and the context. As used in the specification, however, unless specified to the contrary, the following terms have the meaning indicated and the following conventions are adhered to.

Throughout this specification, amino acid positions of the PYY analogues are numbered according to the corresponding position in native human PYY having the sequence shown above.

### PYY Analogues

A PYY analogue is a peptide comprising an amino acid sequence corresponding to the amino acid sequence of hPYY (1-36), hPYY(3-36), or hPYY(4-36). In other words a PYY analogue is a peptide, whose structure is related to PYY, in which one or more amino acid residues have been modified when compared to hPYY (1-36), hPYY(3-36), or hPYY(4-36). Possible modifications are substitutions, insertions, or deletions of amino acids at specific positions. A PYY analogue of the invention relates to a peptide that has retained a certain binding affinity (Ki) towards the hNPY2 receptor.

The term “PYY analogue” comprises the peptide itself, i.e., in a non-ionized state, as well as the peptide in ionized state (e.g. when one or more side chains of its amino acids are ionized, i.e. (de)protonated). A PYY analogue in a non-ionized state is also referred to herein as a non-salt form of the PYY analogue.

The term “PYY analogue” may also refer to peptides in which a half-life extending group is attached to one or more amino acids of the peptide. In such cases, a side chain of an amino acid bears a covalently attached half-life extending group.

The term “PPY analogue” also comprises pharmaceutically acceptable salt forms of the PPY analogue, e.g., when the PPY analogue is in an ionized state.

PYY compounds or PYY analogues of the invention may be described by reference to i) the number of the amino acid residue in hPYY(1-36) (SEQ ID NO:2) which corresponds to the amino acid residue which is changed (i.e., the corresponding position in hPYY(1-36), and to ii) the actual change.

The expressions “a position equivalent to” or “corresponding position” are used to characterise the site of change in a variant PYY sequence by reference to hPYY(1-36).

In general throughout the application, when referring to a particular position of a PYY analogue, the position referred to is the position of the PYY analogue corresponding to that particular position of hPYY(1-36).

In the sequence listing, the first amino acid residue of a given sequence is assigned no. 1. This means that e.g. the first amino acid residue of hPYY(3-36), which is isoleucine, is assigned no. 3 in the sequence listings. This position is also referred to as the position corresponding to position 3 of hPYY(1-36).

As used herein, the term “pharmaceutically acceptable salt” is intended to indicate a salt which is not harmful to a patient or subject to which the salt in question is administered. It may suitably be a salt chosen, e.g., among acid addition salts and basic salts. As used herein, “pharmaceutically acceptable salt” refer to derivatives of the disclosed analogues or compounds wherein the parent analogue or compound is modified by making acid or base salts thereof. Examples of acid addition salts include chloride salts, citrate salts and acetate salts. Examples of basic salts include salts, where the cation is selected among alkali metal cations, such as sodium or potassium ions, alkaline earth metal cations, such as calcium or magnesium ions, as well as substituted ammonium ions, such as ions of the type N(R¹)(R²)(R³)(R⁴)⁺, where R¹, R², R³ and R⁴ independently will typically designate hydrogen or optionally substituted C₁₋₆-alkyl. Other examples of pharmaceutically acceptable salts are described in “Remington's Pharmaceutical Sciences”, 17th edition, Alfonso R. Gennaro (Ed.), Mark Publishing Company, Easton, PA, USA, 1985 (and more recent editions thereof), in the “Encyclopaedia of Pharmaceutical Technology”, 3^{rd} edition, James Swarbrick (Ed.), Informa Healthcare USA (Inc.), NY, USA, 2007, and in J. Pharm. Sci. 66: 2 (1977).

The term “agonist” as employed in the context of the invention refers to a substance that activates the receptor type in question, typically by binding to it (i.e. as a ligand).

Each embodiment of the invention described herein may be taken alone or in combination with one or more other embodiments of the invention.

Throughout the present specification, unless naturally occurring amino acids are referred to by their full name (e.g. alanine, arginine, etc.), they are designated by their conventional three-letter or single-letter abbreviations (e.g. Ala or A for alanine, Arg or R for arginine, etc.).

Unless otherwise indicated, reference is made to the L-isomeric forms of the amino acids in question.

Additional abbreviations include the following:
Hyp: 4-hydroxyproline, e.g. (2S,4R)-4-hydroxyproline [also denoted (4R)-4-hydroxy-L-proline]
γ-Glu: γ-glutamic acid [also denoted gGlu]

The term “C₁₋ₙ alkyl”, wherein n is an integer selected from 2, 3, 4, 5 or 6, either alone or in combination with another radical, denotes an acyclic, saturated, branched or linear hydrocarbon radical with 1 to n C atoms. For example the term C₁₋₅-alkyl embraces the radicals H₃C—, H₃C—CH₂—, H₃C—CH₂—CH₂—, H₃C—CH(CH₃)—, H₃C—CH₂—CH₂—CH₂—, H₃C—CH₂—CH(CH₃)—, H₃C—CH(CH₃)—CH₂—, H₃C—C(CH₃)₂—, H₃C—CH₂—CH₂—CH2-CH2-, H₃C—CH₂—CH₂—CH(CH₃)—, H₃C—CH₂—CH(CH₃)—CH₂—, H₃C—CH(CH₃)—CH₂—CH₂—, H₃C—CH₂—C(CH₃)₂—, H₃C—C(CH₃)₂—CH₂—, H₃C—CH(CH₃)—CH(CH₃)— and H₃C—CH₂—CH(CH₂CH₃)—.

The term “C₃₋ₙ-cycloalkyl”, wherein n is an integer from 4 to n, either alone or in combination with another radical denotes a cyclic, saturated, unbranched hydrocarbon radical with 3 to n C atoms. For example, the term C3-6-cycloalkyl includes cyclopropyl, cyclobutyl, cyclopentyl and cyclohexyl.

“C(O)” or “C(═O)” refers to a carbonyl group.

### Nomenclature of Compounds:

As an example,

(SEQ ID NO: 80)
iVal-APEK(C18DA-gGlu-OEG1-OEG2-)PGEDASPEELQRYYVSL
RHYYNALTRQRY-NH₂,

wherein iVal represents 3-methylbutanoyl (—C(O)CH₂CH(CH₃)₂), C18DA represents 17-carboxyheptadecanoyl,
gGlu represents L-γ-glutamyl, connected via its amino-group to C18DA and via its ycarboxy-group to OEG1, and
OEG1 represents 2-[2-(2-aminoethoxy)ethoxy]acetoyl, connected via its amino-group to gGlu and via its carboxy-group to OEG2, and
OEG2 represents 2-[2-(2-aminoethoxy)ethoxy]acetoyl, connected via its amino-group to OEG1 and via its carboxy-group to F-amino-group of lysine (K),
completely defines the PYY analogue of the following structure (SEQ ID NO: 80):

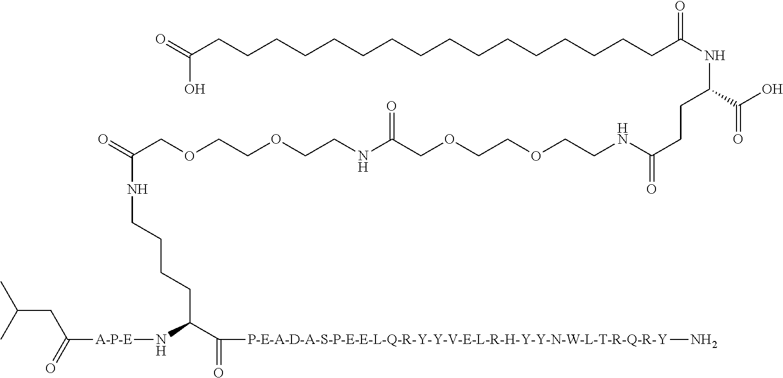

Alternatively, the same compound can be defined in the following way:
N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,18Q,22V,28Y,30A,31L]hPYY(4-36).

The terms “treatment” and grammatical variants thereof (e.g. “treated”, “treating”, “treat”) as employed in the present context refer to an approach for obtaining beneficial or desired clinical results. For the purposes of this invention, beneficial or desired clinical results include, but are not limited to, alleviation of symptoms, diminishment of extent of disease, stabilization (i.e. not worsening) of state of disease, delay or slowing of disease progression, amelioration or palliation of the disease state, and remission (whether partial or total), whether detectable or undetectable. “Treatment” can also mean prolonging survival relative to expected survival time if not receiving treatment. A subject (e.g. a human) in need of treatment may thus be a subject already afflicted with the disease or is disorder in question. The term “treatment” includes inhibition or reduction of an increase in severity of a pathological state or symptoms (e.g. weight gain or hyperglycemia) relative to the absence of treatment and is not necessarily meant to imply complete cessation of the relevant disease, disorder or condition.

The terms “prevention” and grammatical variants thereof (e.g., “prevented”, “preventing”, “prevent”) as employed in the present context refer to an approach for hindering or preventing the development of, or altering the pathology of, a condition, disease or disorder. Accordingly, “prevention” may refer to prophylactic or preventive measures. For the purposes of this invention, beneficial or desired clinical results include, but are not limited to, prevention or slowing of symptoms, progression or development of a disease, whether detectable or undetectable. A subject (e.g. a human) in need of “prevention” may thus be a subject not yet afflicted with the disease or disorder in question. The term “prevention” thus includes inhibiting or slowing the onset of disease relative to the absence of treatment and is not necessarily meant to imply permanent prevention of the relevant disease, disorder or condition.

### Half-Life Extending Group

As described herein, a half-life extending group is covalently attached to a functional group of a side chain of an amino acid of the PYY analogue. The half-life extending group comprises or consists of a lipophilic substituent (X) and optionally a linker (U), wherein one end of the linker U (if present) is attached to an amino acid of the PYY analogue and the other end is connected to the lipophilic substituent (—U—X).

Without wishing to be bound by any particular theory, it is thought that such lipophilic substituents (and other classes of half-life extending moieties) bind albumin and other plasma components in the blood stream, thereby shielding the compound of the invention from renal filtration as well as enzymatic degradation and thus possibly enhancing the half-life of the compound in vivo. The lipophilic substituent may also modulate the potency of the compound as an agonist to the NPY2 receptor or other receptors of the NPY receptor family.

The lipophilic substituent X is attached to the linker U via an ester, ether, a sulfonyl ester, a thioester, an amide, an amine, triazole or a sulfonamide. Accordingly, it will be understood that preferably the lipophilic substituent X includes an acyl group, a sulfonyl group, an alkyne, an azide, an N atom, an O atom or an S atom, which forms part of the ester, sulfonyl ester, thioester, triazole, amide, amine or sulfonamide. Preferably, an acyl group, or an O or N atom in the lipophilic substituent X forms part of an amide or ester with the linker U.

The half-life extending group (the linker U thereof, if present) is attached to an amino acid residue of the PYY analogue via an ester, a sulfonyl ester, a thioester, an amide, an amine or a sulfonamide. Accordingly, it will be understood that preferably the half-life extending group (the linker U thereof, if present) includes an acyl group, a sulfonyl group, an N atom, an O atom or an S atom which forms part of the ester, sulfonyl ester, thioester, amide, amine or sulfonamide. Preferably, an acyl group, or an O or N atom in the linker U forms part of an amide or ester with the amino acid residue.

The lipophilic substituent X may comprise a hydrocarbon chain having from 10 to 24 C atoms, e.g. from 14 to 22 C atoms, e.g. from 16 to 20 C atoms. Preferably, it has at least 14 C atoms, and preferably has 20 C atoms or fewer. For example, the hydrocarbon chain may contain 14, 15, 16, 17, 18, 19 or 20 carbon atoms. The hydrocarbon chain may be linear or branched and may be saturated or unsaturated. Furthermore, it can include a functional group at the end of the hydrocarbon chain, e.g. a carboxylic acid group, a sulphonic acid group, or a tetrazole group. From the discussion above it will also be understood that the hydrocarbon chain is preferably substituted with a moiety, which forms part of the attachment to an amino acid residue of the PYY analogue or to the linker U, for example an acyl group, a sulfonyl group, an N atom, an O atom or an S atom.

Most preferably, the hydrocarbon chain is substituted with an acyl group (for the attachment to the linker U), and accordingly the hydrocarbon chain may be part of an alkanoyl group, for example a dodecanoyl, 2-butyloctanoyl, tetradecanoyl, hexadecanoyl, heptadecanoyl, octadecanoyl, nonadecanoyl or eicosanoyl group. These hydrocarbon chains substituted with an acyl group at one end may further be functionalized with a carboxylic acid group at the other end of the chain. Examples of functionalized hydrocarbon chains (e.g. lipophilic substituents X) are 15-carboxy-pentadecanoyl, 17-carboxy-heptadecanoyl and 19-carboxy-nonadecanoyl.

In certain embodiments, the linker moiety U may itself comprise one, two, three or more linked sub-moieties (U¹, U², U³, etc). In some of these embodiments the linker may comprise one or more (e.g. one, two or three) linked amino acid residues, which may each independently be a residue of any naturally occurring or non-naturally occurring amino acid. For example, the linker may comprise one, two or three linked amino acid residues, each of which may independently be a residue of Gly, Pro, Ala, Val, Leu, Ile, Cys, Phe, Tyr, His, Lys, Arg, Gln, Asn, α-Glu, γ-Glu, ε-Lys, Asp, β-Asp, Ser, Thr, Aib, AEA (2-(2-aminoethoxy)acetic acid), AEEEA (2-{2-[2-(2-aminoethoxy)ethoxy]ethoxy}acetic acid), H₂N-dPEG(4)-COOH (15-amino-4,7,10,13-tetraoxa-pentadecanoic acid), H₂N-dPEG(6)COOH (1-amino-3,6,9,12,15,18-hexaoxahenicosan-21-oic acid), H₂N-dPEG(12)-COOH (1-amino-3,6,9,12,15,18,21,24,27,30,33,36-dodecaoxanonatriacontan-39-oic acid), OEG-OEG (2-[2-(2-{2-[2-(2-aminoethoxy)ethoxy]acetamido}ethoxy)ethoxy]acetic acid), HEbes (3-({2-[2-(2-aminoethoxy)ethoxy]ethyl}carbamoyl)propanoic acid), H-DOOA-DIGOH (2-[({2-[2-(2-aminoethoxy)ethoxy]ethyl}carbamoyl)methoxy]acetic acid), H-TTDDIG-OH (2-{[(3-{2-[2-(3-aminopropoxy)ethoxy]ethoxy}propyl)carbamoyl]methoxy}acetic acid), H-TTDS-OH (3-[(3-{2-[2-(3-aminopropoxy)ethoxy]ethoxy}propyl)carbamoyl]propanoic acid), or 8Ado (i.e. 8-amino-3,6-dioxaoctanoyl also denoted OEG herein).

References to γ-Glu, ε-Lys, and β-Asp indicate residues of amino acids which participate in bonds via their side chain carboxyl or amine functional groups. Thus γ-Glu, and β-Asp participate in bonds via their alpha amino and side chain carboxyl groups, while ε-Lys participates via its carboxyl and side chain amino groups. In the context of the present invention, γ-Glu, gGlu and isoGlu are used interchangeably.

In certain embodiments, the linker U comprises or consists of one, two or three independently selected sub-moieties (U¹, U², U³) selected from the group consisting of Ala, Glu, γ-Glu, Gly, ε-Lys, Ser, OEG and OEG-OEG.

Linkers consisting of γ-Glu, γ-Glu-γ-Glu, γ-Glu-OEG, OEG-OEG, γ-Glu-γ-Glu-OEG, γ-Glu-OEG-OEG may be preferred.

### Synthesis of PYY Analogues

The invention provides a method of synthesis of a PYY analogue of the invention. The PYY analogues may be manufactured by standard synthetic methods, including standard solid-phase or liquid-phase methodology. Peptides are assembled either stepwise or by merging fragments, and optionally isolated and purified yielding the final peptide product. Synthesis examples are described in numerous publications, including Fields, G. B. et al., “Principles and Practice of Solid-Phase Peptide Synthesis” in Synthetic Peptides, Grant G. A. (ed.), Oxford University Press (2^{nd} edition, 2002).

### Embodiments

In a first aspect, the invention provides a PYY analogue, wherein the analogue comprises
i) alanine at the position corresponding to position 4 of hPYY(1-36)
ii) lysine at the position corresponding to position 7 of hPYY(1-36)
iii) the sequence QRY at its C-terminal end,
and wherein a half-life extending group is attached to the epsilon amino group of the lysine at position 7 or of a lysine at positions 6, 10, 11, 14, 17, 21, 22 or 30, or to the carboxylic acid group of the side chain of a aspartate or a glutamate at positions 14 or 30.

In some embodiments, the PYY analogue comprises glutamate at the position corresponding to position 9 of hPYY(1-36).

In some embodiments, the PYY analogue comprises tyrosine at the position corresponding to position 20 of hPYY(1-36).

In some embodiment, the PYY analogue comprises tryptophan at the position corresponding to position 30 of hPYY(1-36).

In some embodiments, the PYY analogue comprises leucine at the position corresponding to position 31 of hPYY(1-36).

In some embodiments, the PYY analogue comprises arginine or lysine at the position corresponding to position 33 of hPYY(1-36).

In some embodiments, the PYY analogue bears a half-life extending group, which is attached to the epsilon amino group of the lysine at position 7.

In some embodiments of the present invention, the half-life extending group consists of a lipophilic substituent X and a linker U, wherein the linker U is attached to the amino acid side chain, and X is attached to U.

In some embodiments, the lipophilic substituent X is selected from the group consisting of 15-carboxy-pentadecanoyl, 17-carboxy-heptadecanoyl (C18DA) and 19-carboxy-nonadecanoyl.

In some embodiments the linker U comprises one or more sub-moieties, wherein at least one of the sub-moieties is OEG.

In some embodiments the linker U consists of one, two or three sub-moieties (U1, U2, U3), wherein the sub-moiety is independently selected from the group consisting of Gly, Glu, γ-Glu, ε-Lys, Ser and OEG.

In some embodiments, the PYY analogue comprises
i) alanine at the position corresponding to position 4 of hPYY(1-36)
ii) lysine at the position corresponding to position 7 of hPYY(1-36)
iii) glutamate at the position corresponding to position 9 of hPYY(1-36)
iv) tyrosine at the position corresponding to position 20 of hPYY(1-36)
v) tryptophan at the position corresponding to position 30 of hPYY(1-36)
vi) leucine at the position corresponding to position 31 of hPYY(1-36)
vii) the sequence RQRY (SEQ ID NO: 258) at its C-terminal end,
and wherein a half-life extending group is attached to the epsilon amino group of the lysine at position 7 or of a lysine at positions 6, 10, 11, 14, 17, 21 or 22, or to the carboxylic acid group of the side chain of a aspartate or a glutamate at position 14.

In some embodiments, the PYY analogue comprises threonine at the position corresponding to position 32 of hPYY(1-36).

In some embodiments, the PYY analogue comprises the sequence WLTRQRY (SEQ ID NO: 259) at its C-terminal end.

In some embodiments, the PYY analogue bears a half-life extending group, which is attached to the epsilon amino group of the lysine at position 7.

In some embodiments, the PYY analogue comprises
i) alanine at the position corresponding to position 4 of hPYY(1-36)
ii) lysine at the position corresponding to position 7 of hPYY(1-36)
iii) glutamate at the position corresponding to position 9 of hPYY(1-36)
iv) tyrosine at the position corresponding to position 20 of hPYY(1-36)
v) the sequence WLTRQRY (SEQ ID NO: 259) at its C-terminal end,
and wherein a half-life extending group is attached to the epsilon amino group of the lysine at position 7 or of a lysine at positions 6, 10, 11, 14, 17, 21, or 22, or to the carboxylic acid group of the side chain of a aspartate or a glutamate at position 14.

In some embodiments, the PYY analogue comprises arginine at the position corresponding to position 25 of hPYY(1-36).

In some embodiments, the PYY analogue bears a half-life extending group, which is attached to the epsilon amino group of the lysine at position 7.

In some embodiments, exactly one half-life extending group is attached to the PYY analogue, said half-life extending group being attached to the epsilon amino group of the lysine at position 7.

In some embodiments of the present invention, the PYY analogue is a compound having the formula:

R¹—Z—R²,
wherein R¹ is hydrogen, —C(O)C₁₋₆ alkyl, —C(O)C₆H₆, —C(O)C₃₋₆ cycloalkyl, —C(O)C₁₋₆ alkyl-C₃₋₆ cycloalkyl, C₁₋₆ alkyl or C₁₋₆ alkyl-C₃₋₆ cycloalkyl:
R² is OH or NHR³, wherein R³ is hydrogen or C₁₋₃ alkyl; and
Z is a peptide comprising an amino acid sequence of formula I (SEQ ID NO: 254):

Ala-X5-X6-Lys-X8-X9-X10-X11-X12-X13-X14-X15-X16-X17-X18-X19-Tyr-X21-X22-X23-X24-Arg-X26-X27-X28-X29-X30-X31-X32-Arg-Gln-Arg-Tyr (I)
wherein
X5 is selected from the group consisting of Pro and Hyp;
X6 is selected from the group consisting of Ala and Glu;
X8 is selected from the group consisting of Ala, Ile, Pro, Thr, Val and Hyp;
X9 is selected from the group consisting of Glu, Gly, Gln and Pro;
X10 is selected from the group consisting of Ala and Glu;
X11 is selected from the group consisting of Ala, Asp, Glu and Pro;
X12 is selected from the group consisting of Ala, Gly, Ser, Thr and Val;
X13 is selected from the group consisting of Ala, Glu, Ser, Gln, Thr and Pro;
X14 is selected from the group consisting of Ala, Glu, Gly, Pro and Hyp;
X15 is selected from the group consisting of Ala, Glu and Ser;
X16 is selected from the group consisting of Ala, Glu and Ser;
X17 is selected from the group consisting of Ala, Ile, Leu, Thr and Val;
X18 is selected from the group consisting of Glu and Gln;
X19 is selected from the group consisting of Ala, Glu, Arg, Lys and Gln;
X21 is selected from the group consisting of Ala, Glu, Gln and Tyr;
X22 is selected from the group consisting of Ile, Ser, Thr and Val;
X23 is selected from the group consisting of Ala, Glu, Ser and Thr;
X24 is selected from the group consisting of Ala, Ile, Leu, Thr and Val;
X26 is selected from the group consisting of Ala, His and Lys;
X27 is selected from the group consisting of Gln and Tyr;
X28 is selected from the group consisting of His, Trp and Tyr;
X29 is selected from the group consisting of Asn, Trp and Tyr;
X30 is selected from the group consisting of Ala, His, Trp, and Tyr;
X31 is selected from the group consisting of Ala, Ile, Leu and Thr;
X32 is selected from the group consisting of Gln, Leu and Thr;
wherein one to three amino acids of X5, X6, X8-X19, X21-X24 and X26-X32 may be absent,
and wherein a half-life extending group is attached to the epsilon amino group of the lysine at position 7.

In a further embodiment the PYY analogue is according to the previous embodiment, wherein none of X5, X6, X8-X19, X21-X24 and X26-X32 is absent.

In a further embodiment R¹ is —C(O)C₁₋₄ alkyl, —C(O)C₃₋₅ cycloalkyl, —C(O)C₁₋₃ alkylC₃₋₄ cycloalkyl, C₁₋₄ alkyl or C₁₋₃ alkyl-C₃₋₄ cycloalkyl.

In a further embodiment R¹ is —C(O)CH₂CH(CH₃)₂, —C(O)CH₂-cyclobutyl, —C(O)CH₂-cyclopropyl.

It has been found that the PYY analogues of the present invention—bearing alanine at position 4 and lysine at position 7—generally are soluble around pH 6 and pH 7.

Peptide therapeutics are usually provided as pharmaceutical liquid formulation in a prefilled ready-to-use injection device. These peptide formulations for subcutaneous administration have limited application volumes. Therefore, good solubility of the peptides is a requirement for the application in a ready-to-use injection device.

A further important aspect is the long-term stability and solubility of the peptides in the liquid formulation. A property of fundamental importance for physical stability is the intrinsic solubility (at a given pH value).

A broad pH range, within which a peptide therapeutic is reasonably soluble (solubility window), is also desirable as it allows more flexibility for pharmaceutical formulation development. This flexibility might be desirable as other factors, such as chemical stability, are also pH dependent. In general, a peptide formulation around pH 6.0 is believed to show reduced rates of oxidation (e.g. Cys oxidation, disulphide crosslinking, and oxidation of Trp residues), deamination and aspartate isomerization as compared to a formulation at pH 7. For instance, Bak et al. (A. Bak, D. Leung, S. E. Barrett, S. Forster, E. D. Minnihan, A. W. Leithead, J. Cunningham, N. Toussaint, L. S. Crocker, The AAPS Journal, Vol. 17, No. 1, 2015, p. 144-155) states that oxidation propensity generally lessens at lower pH and suggests maintenance at pH<7 as a strategy for mitigation risks related to oxidation. Therefore, it might be desirable to have the option to formulate around pH 6 in case chemical stability of peptides containing asparagine, aspartate or glutamine, tryptophan, cysteine or methionine is an issue.

Efficacy of obesity therapeutics is limited. However, efficacy might be improved by combining different therapeutic principles. NPY2 receptor agonists seem attractive partners for combination with other weight reducing therapeutics. For example, NPY2 receptor agonists show enhanced weight loss efficacy with GLP-1 receptor agonists (e.g. WO2005/077072, WO2014/178018, WO2018/081370) or amylin (e.g. WO2006/066024, WO2009/064298). Native amylin and many amylin (or calcitonin) analogues comprise a disulphide bridge. Therefore, a fix-dose combination of a PYY analogue with an amylin analogue might benefit from the opportunity to formulate at a lower pH where the disulphide bridge of the amylin analogue potentially shows improved stability (e.g. due to reduced intramolecular disulphide bond crosslinking reactions). This illustrates that the advantage to be able to formulate at a pH below 7 may not (only) be due to a higher stability of the NPY analogue but may lie therein that the combination partner shows improved stability (or solubility) at a lower pH.

Therefore, to enable co-formulation development with a diverse range of partners, it is highly desirable to identify NPY2 receptor agonists with a wide solubility window.

Increased solubility, however, should not come at the cost of reduced activity or potency. It was surprisingly found that the alanine at position 4 increases solubility around pH 6 in the PYY analogues of the invention with no or only small negative effects on activity or potency or other important properties (e.g. chemical or physical stability).

Long in-vivo half-life is also a beneficial property for agents to reduce food intake in overweight or obese patients. Compounds with a long-acting profile (as compared to the very short in-vivo half-life of native (human) PYY) reducing the frequency of administration are desirable.

In one aspect, the invention relates to PYY analogues being NPY2 receptor agonists. In one aspect, the invention relates to PYY analogues showing selectivity towards the NPY receptor subtype Y2 as compared to Y receptor subtypes Y1, Y4 and Y5.

In one aspect, the invention relates to PYY analogues with extended half-live, e.g. with longer half-life than the half-life of hPYY(3-36). For example, the PYY analogues of the invention are suitable for once weekly administration.

Additionally or alternatively, the invention relates to PYY analogues having high chemical and/or physical stability, e.g. around pH 6 or pH 7.

Further, more specific embodiments are defined below:

In a further embodiment of the present invention, the PYY analogue is a compound having the formula:

R¹—Z—R²,
wherein R¹ is hydrogen, —C(O)C₁₋₆ alkyl, —C(O)C₆H₆, —C(O)C₃₋₆ cycloalkyl, —C(O)C₁₋₆ alkyl-C₃₋₆ cycloalkyl, or C₁₋₆ alkyl or C₁₋₆ alkyl-C₃₋₆ cycloalkyl:
R² is OH or NHR³, wherein R³ is hydrogen or C₁₋₃ alkyl; and
Z is an amino acid sequence of formula II (SEQ ID NO: 255):

Ala-X5-X6-Lys-X8-X9-X10-X11-X12-X13-X14-X15-X16-X17-X18-X19-Tyr-X21-X22-X23-X24-Arg-X26-Tyr-X28-X29-X30-X31-X32-Arg-Gln-Arg-Tyr (II)
wherein
X5 is selected from the group consisting of Pro and Hyp;
X6 is selected from the group consisting of Ala and Glu;
X8 is selected from the group consisting of Ala, Pro and Hyp;
X9 is selected from the group consisting of Gln, Gly, Glu and Pro;
X10 is selected from the group consisting of Ala and Glu;
X11 is selected from the group consisting of Ala, Asp, Glu and Pro;
X12 is selected from the group consisting of Ala, Gly and Ser;
X13 is selected from the group consisting of Ala, Glu, Ser, Thr and Pro;
X14 is selected from the group consisting of Ala, Glu, Pro and Hyp;
X15 is selected from the group consisting of Ala, Glu and Ser;
X16 is selected from the group consisting of Ala, Glu and Ser;
X17 is selected from the group consisting of Ile, Leu, Thr and Val;
X18 is selected from the group consisting of Gln and Glu;
X19 is selected from the group consisting of Ala, Glu, Arg, Lys and Gln
X21 is selected from the group consisting of Ala, Glu, Gln and Tyr;
X22 is selected from the group consisting of Ile, Ser, Thr and Val;
X23 is selected from the group consisting of Ala, Glu, Ser and Thr;
X24 is selected from the group consisting of Ala, Ile, Leu, Thr and Val;
X26 is selected from the group consisting of Ala, His and Lys;
X28 is selected from the group consisting of Trp and Tyr;
X29 is selected from the group consisting of Asn and Tyr;
X30 is selected from the group consisting of His, Trp, and Tyr;
X31 is selected from the group consisting of Ala, Ile and Leu;
X32 is selected from the group consisting of Gln and Thr;
and wherein a half-life extending group is attached to the epsilon amino group of the lysine at position 7.

In some embodiments in line with the embodiment described immediately above, the half-life extending group consists of a lipophilic substituent X and a linker U, wherein the linker U is attached to the amino acid side chain, and X is attached to U, and wherein the lipophilic substituent X is selected from the group consisting of 15-carboxy-pentadecanoyl, 17-carboxy-heptadecanoyl (C18DA) and 19-carboxy-nonadecanoyl, and the linker U consists of one, two or three sub-moieties (U1, U2, U3), wherein the sub-moiety is independently selected from the group consisting of Gly, Glu, γ-Glu, ε-Lys, Ser and OEG.

In some embodiments of the present invention, the PYY analogue is a compound having the formula:

R′—Z—R²,
wherein R¹ is hydrogen, —C(O)C₁₋₆ alkyl, —C(O)C₆H₆, —C(O)C₃₋₆ cycloalkyl, —C(O)C₁₋₆ alkyl-C₃₋₆ cycloalkyl, or C₁₋₆ alkyl or C₁₋₆ alkyl-C₃₋₆ cycloalkyl:
R² is OH or NHR³, wherein R³ is hydrogen or C₁₋₃ alkyl; and
Z is an amino acid sequence of formula III (SEQ ID NO: 256):

Ala-Pro-X6-Lys-Pro-X9-X10-X11-X12-X13-X14-X15-X16-X17-X18-X19-Tyr-X21-Valis X23-Leu-Arg-His-Tyr-Tyr-Asn-Trp-Leu-Thr-Arg-Gln-Arg-Tyr (III)
wherein
X6 is selected from the group consisting of Ala and Glu;
X9 is selected from the group consisting of Glu and Gly;
X10 is selected from the group consisting of Ala and Glu;
X11 is selected from the group consisting of Ala, Asp, Glu and Pro;
X12 is selected from the group consisting of Ala and Ser;
X13 is selected from the group consisting of Ala, Glu, Ser, Thr and Pro;
X14 is selected from the group consisting of Ala, Glu and Pro;
X15 is selected from the group consisting of Ala and Glu,
X16 is selected from the group consisting of Ala and Glu;
X17 is selected from the group consisting of Ile, Leu, Thr and Val;
X18 is selected from the group consisting of Glu and Gln;
X19 is selected from the group consisting of Ala, Glu, Arg, Lys, and Gln;
X21 is selected from the group consisting of Glu and Tyr;
X23 is selected from the group consisting of Ala, Glu, Ser and Thr;
and wherein a half-life extending group is attached to the epsilon amino group of the lysine at position 7.

In further embodiments, the PYY analogue is a compound according to formula I to III, wherein at least two amino acids from X6, X10, X11, X13 and X23 are selected from the group consisting of Asp and Glu.

In further embodiments, the PYY analogue is a compound according to any of the previous embodiments, wherein only one of X6, X10 and X15 is Ala.

In a further embodiment of the present invention, the PYY analogue is a compound having the formula:

R¹—Z—R²,
wherein R¹ is hydrogen, —C(O)C₁₋₆ alkyl, —C(O)C₆H₆, —C(O)C₃₋₆ cycloalkyl, —C(O)C₁₋₆ alkyl-C₃₋₆ cycloalkyl, or C₁₋₆ alkyl or C₁₋₆ alkyl-C₃₋₆ cycloalkyl:
R² is OH or NHR³, wherein R³ is hydrogen or C₁₋₃ alkyl; and
Z is an amino acid sequence of formula IV (SEQ ID NO: 257):

Ala-Pro-X6-Lys-Pro-Glu-X10-X11-Ala-X13-X14-Glu-Glu-X17-Gln-X19-Tyr-Tyr-Val-X23-Leu-Arg-His-Tyr-Tyr-Asn-Trp-Leu-Thr-Arg-Gln-Arg-Tyr (IV)
wherein
X6 is selected from the group consisting of Ala and Glu;
X10 is selected from the group consisting of Ala and Glu;
X11 is selected from the group consisting of Ala, Asp and Glu;
X13 is selected from the group consisting of Ala, Glu, Ser, and Thr;
X14 is selected from the group consisting of Ala, Pro and Hyp;
X17 is selected from the group consisting of Ile and Leu;
X19 is selected from the group consisting of Arg, Lys, and Gln;
X23 is selected from the group consisting of Ala, Glu, and Ser;
and wherein a half-life extending group is attached to the epsilon amino group of the lysine at position 7.

In a further embodiment, the PYY analogue is a compound according to formula IV, wherein at least 2 amino acid from X6, X10, X11, X13 and X23 are selected from Asp or Glu.

In further embodiments, the PYY analogue is a compound according to formula IV, wherein only one of X6, and X10 is Ala.

In further embodiments, the PYY analogue is a compound according to any of the previous embodiments, wherein X6 is Ala.

In further embodiments, R¹ is hydrogen, —C(O)C₁₋₆ alkyl, —C(O)C₃₋₆ cycloalkyl, or C₁₋₆ alkyl. In further embodiments, R¹ is hydrogen or —C(O)C₁₋₆ alkyl.

In further embodiments, R¹ is hydrogen or —C(O)C₁₋₄ alkyl.

In more specific embodiments, R¹ is —C(O)CH₂CH(CH₃)₂.

In more specific embodiments, R² is NH₂.

In some embodiments of the present invention, the PYY analogue is a compound having the formula:

R¹—Z—R²,
wherein R¹ and R² are defined as in any of the definitions above; and
Z is an amino acid sequence selected from Table 1:

TABLE 1

| | | SEQ ID |
|---|---|---|
| Compound | Sequence | NO |
| 1 | APEKPEEDAQPEELQEYYVSLRHYYNWLTRQRY | 5 |
| 2 | AHypEKPEEDASPEELQRYYVSLRHYYNWLTRQRY | 6 |
| 3 | APEKPEEEASPAELQRYYVSLRHYYNWLTRQRY | 7 |
| 4 | APEKPEEDASPEELQRYYVSLRHYYNWLQRQRY | 8 |
| 5 | APEKPEEEVSPEELQRYYVSLRHYYNWLTRQRY | 9 |
| 6 | APEKPEEEASPEELQRYYVSARHYYNWLTRQRY | 10 |
| 7 | APEKPEEDASPEELQAYYVSLRHYYNWLTRQRY | 11 |
| 8 | APEKPEEDASEEELQQYYVSLRHYYNWLTRQRY | 12 |
| 9 | APEKPEEDASPEEIQRYYVELRHYYNWLTRQRY | 13 |
| 10 | APEKPEADASPEETQRYYVSLRHYYNWLTRQRY | 14 |
| 11 | APEKPEEDASPEEIQQYYVSLRHYYNWLTRQRY | 15 |
| 12 | APEKPEEDAPGEELQRYYVSLRHYYNWLTRQRY | 16 |
| 13 | APEKPEEDAAPEELQQYYVSLRHYYNWLTRQRY | 17 |
| 14 | APEKPEADATPEELQRYYVSLRHYYNWLTRQRY | 18 |
| 15 | APEKPEEDASPEEIQRYEVSLRHYYNWLTRQRY | 19 |
| 16 | APEKPEEDASPEETQRYYVTLRHYYNWLTRQRY | 20 |
| 17 | APEKPEEDASPAELQRYYVSLRHYYNWLTRQRY | 21 |
| 18 | APEKPEEDAEPEELQQYYVSLRHYYNWLTRQRY | 22 |
| 19 | APEKPEADASPEELQQYYVSLRHYYNWLTRQRY | 23 |
| 20 | APEKPEEDAAPEELQRYYVELRHYYNWLTRQRY | 24 |
| 21 | APEKPEEDASPEELQKYYVSLRHYYNWLTRQRY | 25 |
| 22 | APEKPEEDASPEELQRYYTSLRHYYNWLTRQRY | 26 |
| 23 | APAKPEEDASPEEIQRYYVSLRHYYNWLTRQRY | 27 |
| 24 | APEKPEEDASPEELQRYYVSLRKYYNWLTRQRY | 28 |
| 25 | APEKPEEEASAEELQRYYVSLRHYYNWLTRQRY | 29 |
| 26 | APEKPPEDATPEELQRYYVSLRHYYNWLTRQRY | 30 |
| 27 | APEKPPEDAEPEELQRYYVSLRHYYNWLTRQRY | 31 |
| 28 | APEKPEEDTSPEELQRYYVELRHYYNWLTRQRY | 32 |
| 29 | APEKPEAPASPEELQRYYVELRHYYNWLTRQRY | 33 |
| 30 | APEKPEADASEEELQRYYVSLRHYYNWLTRQRY | 34 |
| 31 | APEKPEEDASPEETQRYYVALRHYYNWLTRQRY | 35 |
| 32 | APEKPEEDTSPEELQRYEVSLRHYYNWLTRQRY | 36 |
| 33 | APEKPEEPASPEELQRYYVELRHYYNWLTRQRY | 37 |
| 34 | APEKPEEDASPEELQRYYVSTRHYYNWLTRQRY | 38 |
| 35 | APEKPEEDAAPEETQRYYVSLRHYYNWLTRQRY | 39 |
| 36 | APEKAEEDAEPEELQRYYVSLRHYYNWLTRQRY | 40 |
| 37 | APEKPEEDASPEETQRYEVSLRHYYNWLTRQRY | 41 |
| 38 | APEKPPEDASPEELQRYYVALRHYYNWLTRQRY | 42 |
| 39 | APEKPPADAEPEELQRYYVSLRHYYNWLTRQRY | 43 |
| 40 | APEKPEEDATPEETQRYYVSLRHYYNWLTRQRY | 44 |
| 41 | APEKPEEEATPEELQRYYVSLRHYYNWLTRQRY | 45 |
| 42 | APEKPEEAASPEELQRYYVSTRHYYNWLTRQRY | 46 |
| 43 | APEKPEAPASPEELQRYYVSLRHYYNWLTRQRY | 47 |
| 44 | APEKPEEEASPEELQRYYVSLRHYYNWITRQRY | 48 |
| 45 | APEKPEEEASPESLQRYYVSLRHYYNWLTRQRY | 49 |
| 46 | APEKPEEEASPEELQRYYVSLRHYYNWLTRQRY | 50 |
| 47 | APEKPPADASPEELQRYYVSLRHYYNWLTRQRY | 51 |
| 48 | APEKPEEDTEPEELQRYYVSLRHYYNWLTRQRY | 52 |
| 49 | APEKPEEDASPEELQQYYVELRHYYNWLTRQRY | 53 |
| 50 | APEKPEEDASAEELQRYYVSLRHYYNWITRQRY | 54 |
| 51 | APEKAEEEASPEELQRYYVSLRHYYNWLTRQRY | 55 |
| 52 | APEKAEEDASEEELQRYYVSLRHYYNWLTRQRY | 56 |
| 53 | APEKPEADASPEEIQRYYVSLRHYYNWLTRQRY | 57 |
| 54 | APEKPEEDASPEELQRYYVSLRHYYNWLTRQRY | 58 |
| 55 | APEKPEEDASPEEAQRYYVSLRHYYNWLTRQRY | 59 |
| 56 | APEKPEADAEPEELQRYYVSLRHYYNWLTRQRY | 60 |
| 57 | APEKPEEDASAEEIQRYYVSLRHYYNWLTRQRY | 61 |
| 58 | APEKPEEDASPEELQQYYVSLRHYYNWLTRQRY | 62 |
| 59 | APEKPEEDASPEETEQYYVSLRHYYNWLTRQRY | 63 |
| 60 | APEKPEEAASPEELEQYYVSLRHYYNWLTRQRY | 64 |
| 61 | APEKPPEDASPEEIQRYYVSLRHYYNWLTRQRY | 65 |
| 62 | APEKPGEDASPEELERYYVSLRHYYNWLTRQRY | 66 |
| 63 | APEKPEEDASPEELQRYYVSLRAYYNWLTRQRY | 67 |
| 64 | APEKPGEDASPEELQRYYVSLRHYYNWLTRQRY | 68 |
| 65 | APEKPEEDASPEELQRYYVEIRHYYNWLTRQRY | 69 |
| 66 | APEKPEEDASAEETQRYYVSLRHYYNWLTRQRY | 70 |
| 67 | APEKPEEDSAPEELQRYYVSLRHYYNWLTRQRY | 71 |
| 68 | APEKPEEEASPEELQAYYVSLRHYYNWLTRQRY | 72 |
| 69 | APEKPEEDSSAEELQRYYVSLRHYYNWLTRQRY | 73 |
| 70 | APEKPEEAASPEELQRYEVSLRHYYNWLTRQRY | 74 |
| 71 | APEKPEEEASPSELQRYYVSLRHYYNWLTRQRY | 75 |
| 72 | APEKPEEEGSPEELQRYYVSLRHYYNWLTRQRY | 76 |
| 73 | APEKPEEEASPEELQRYYVSLRAYYNWLTRQRY | 77 |
| 74 | APEKPEEDASAEELQRYYVALRHYYNWLTRQRY | 78 |
| 75 | APEKPEEDASAEELQRYYVTLRHYYNWLTRQRY | 79 |
| 76 | APEKPGEDASPEELQRYYVSLRHYYNALTRQRY | 80 |
| 77 | APEKPEEDASEEELQRYYVELRHYYNWLTRQRY | 81 |
| 78 | APEKPEEDAEPEELQQYYVTLRHYYNWLTRQRY | 82 |
| 79 | APEKPEEEASEEELQRYYVSLRHYYNWLTRQRY | 83 |
| 80 | APEKPEEEASPEETQRYYVSLRHYYNWLTRQRY | 84 |
| 81 | APEKPEEDASPEELQRYYVSIRHYYNWLTRQRY | 85 |
| 82 | APEKPEEDASPEALQRYYVALRHYYNWLTRQRY | 86 |
| 83 | APEKPEADASPAELQRYYVSLRHYYNWLTRQRY | 87 |
| 84 | APEKPEEDASPEEIQRYYVSLRHYYNWLTRQRY | 88 |
| 85 | APEKPEEDASPEELQRYAVSLRHYYNWLTRQRY | 89 |
| 86 | APEKPEADASPEELQRYEVSLRHYYNWLTRQRY | 90 |
| 87 | APEKPEEDSSPAELQRYYVSLRHYYNWLTRQRY | 91 |
| 88 | APEKPPEDASPEELQRYYVELRHYYNWLTRQRY | 92 |
| 89 | APEKPEEEAEPEELQRYYVSLRHYYNWLTRQRY | 93 |
| 90 | APEKPEEDASPEELQRYEVSLRHYYNWLTRQRY | 94 |
| 91 | APEKPEEDAPAEELQRYYVELRHYYNWLTRQRY | 95 |
| 92 | APEKPEEDTSPEELQRYYVSLRHYYNWLTRQRY | 96 |
| 93 | APEKPEEDASAEELQKYYVSLRHYYNWLTRQRY | 97 |
| 94 | APEKPEEDASPEETQRYYVELRHYYNWLTRQRY | 98 |
| 95 | APEKPEEDASPAEIQRYYVSLRHYYNWLTRQRY | 99 |
| 96 | APEKPEADASPEELQRYYVSLRHYYNWLTRQRY | 100 |
| 97 | APEKPEEDASPEELQRYYVALRHYYNWLTRQRY | 101 |
| 98 | APEKPEEDASPEELQRYYSALRHYYNWLTRQRY | 102 |
| 99 | APEKPEEDAEAEELQRYYVSLRHYYNWLTRQRY | 103 |
| 100 | APAKPPEDASPEELQRYYVSLRHYYNWLTRQRY | 104 |
| 101 | APEKPEEDATPEELQRYYVELRHYYNWLTRQRY | 105 |
| 102 | APEKPEEDASPEELQRYYVTLRHYYNWLTRQRY | 106 |
| 103 | APAKPEEDASEEELQRYYVSLRHYYNWLTRQRY | 107 |
| 104 | APEKPEEDASAEELQRYYVSLRHYYNWLTRQRY | 108 |
| 105 | APEKPEEDAEPEELQEYYVSLRHYYNWLTRQRY | 109 |
| 106 | APAKPEEDATPEELQRYYVSLRHYYNWLTRQRY | 110 |
| 107 | APEKPEEDASPEELQKYYVALRHYYNWLTRQRY | 111 |
| 108 | APEKPEEDASPEELQRYYVELRHYYNWLTRQRY | 112 |
| 109 | APEKPEEAASPEELQRYYVSLRHYYNWLTRQRY | 113 |
| 110 | APEKPEEDAEPEEIQRYYVSLRHYYNWLTRQRY | 114 |
| 111 | APEKPEEDAEPEELQRYYVELRHYYNWLTRQRY | 115 |
| 112 | APEKPEEDATPEELQRYYVSLRHYYNWLTRQRY | 116 |
| 113 | APEKAEEDATPEELQRYYVSLRHYYNWLTRQRY | 117 |
| 114 | APEKPEEDASPEELQRYYVSLRHYHWYLTRQRY | 118 |
| 115 | APEKPEEEASPEALQRYYVSLRHYYNWLTRQRY | 119 |
| 116 | APEKPEEDAAAEELQRYYVSLRHYYNWLTRQRY | 120 |
| 117 | APAKPEEDASPEELQRYYVALRHYYNWLTRQRY | 121 |
| 118 | APEKPEEDASAAELQRYYVSLRHYYNWLTRQRY | 122 |
| 119 | APEKPEEEASPEELQRYYVTLRHYYNWLTRQRY | 123 |
| 120 | APEKPGEDAEEEELQEYYVSLRHYYNWLTRQRY | 124 |
| 121 | APAKPEADASPEELQRYYVSLRHYYNWLTRQRY | 125 |
| 122 | APEKPEEDAAPEELQRYYVSLRHYYNWLTRQRY | 126 |
| 123 | APEKPEEDASPEELQRYYVSLRHYWYHLTRQRY | 127 |
| 124 | APEKPEEDAAPEEIQRYYVSLRHYYNWLTRQRY | 128 |
| 125 | APEKPGEDASPEELQRYYVSLRHYYNWATRQRY | 129 |
| 126 | APAKPEEDSSPEELQRYYVSLRHYYNWLTRQRY | 130 |
| 127 | APAKPEEDASAEELQRYYVSLRHYYNWLTRQRY | 131 |
| 128 | APEKPEEDAPAEEIQRYYVSLRHYYNWLTRQRY | 132 |
| 129 | APEKPEEDASPEETQRYYTALRHYYNWLTRQRY | 133 |
| 130 | APEKPEEDTSAEELQRYYVSLRHYYNWLTRQRY | 134 |
| 131 | APEKAEEDASPEELQRYYVELRHYYNWLTRQRY | 135 |
| 132 | APEKPEEDASPEELQKYYVTLRHYYNWLTRQRY | 136 |
| 133 | APEKPEEDASPEEVQRYYVELRHYYNWLTRQRY | 137 |
| 134 | APEKPEEDASPEEIQRYYTELRHYYNWLTRQRY | 138 |
| 135 | APEKPEADASPEELQRYYVELRHYYNWLTRQRY | 139 |
| 136 | APEKPEADSSPEELQRYYVSLRHYYNWLTRQRY | 140 |
| 137 | APEKPEEDASPEELQRYYVSLRHYYNWITRQRY | 141 |
| 138 | APAKPEEDASPEELQQYYVSLRHYYNWLTRQRY | 142 |
| 139 | APEKPEEESSPEELQRYYVSLRHYYNWLTRQRY | 143 |
| 140 | APEKPGEDASPEELEQYYVSLRHYYNWLTRQRY | 144 |
| 141 | APEKPEEAASPEELQRYYVSARHYYNWLTRQRY | 145 |
| 142 | APEKPPEEASPEELQRYYVSLRHYYNWLTRQRY | 146 |
| 143 | APEKPEEDATPEELQRYYVALRHYYNWLTRQRY | 147 |
| 144 | APEKAEEDASPEELQQYYVSLRHYYNWLTRQRY | 148 |
| 145 | APEKPEEDSSPEEIQRYYVSLRHYYNWLTRQRY | 149 |
| 146 | APEKPEEEASPEELQRYYVSTRHYYNWLTRQRY | 150 |
| 147 | APEKPEEDASPEETQKYYVSLRHYYNWLTRQRY | 151 |
| 148 | APEKPEEDAPAEELQRYYVSLRHYYNWLTRQRY | 152 |
| 149 | APEKPEEAASPEELQRYYVELRHYYNWLTRQRY | 153 |
| 150 | APEKPEEDAEPEELQKYYVSLRHYYNWLTRQRY | 154 |
| 151 | APEKPEAEASPEELQRYYVSLRHYYNWLTRQRY | 155 |
| 152 | APEKAEEDASPEEIQRYYVSLRHYYNWLTRQRY | 156 |
| 153 | APEKVEEEASPEELQRYYVSLRHYYNWLTRQRY | 157 |
| 154 | APEKPEEEASPEELQRYEVSLRHYYNWLTRQRY | 158 |
| 155 | APEKPEEDASPEETQRYYVSLRHYYNWLTRQRY | 159 |
| 156 | APEKPEEDASEEELEQYYVSLRHYYNWLTRQRY | 160 |
| 157 | APAKPEEDASPEETQRYYVSLRHYYNWLTRQRY | 161 |
| 158 | APEKPEEDASPEELQRYYVSLRHYWNYLTRQRY | 162 |
| 159 | APEKPEADASPEELEQYYVSLRHYYNWLTRQRY | 163 |
| 160 | APEKPEADAEPEELQRYYTSLRHYYNWLTRQRY | 164 |
| 161 | APEKPEEDATPEELQQYYVSLRHYYNWLTRQRY | 165 |
| 162 | APEKPEEDASPEELQRYEVSLRAYYNWLTRQRY | 166 |
| 163 | APEKPEEAASPEELQRYYVALRHYYNWLTRQRY | 167 |
| 164 | APEKPEEDASEEELQEYYVSLRHYYNWLTRQRY | 168 |
| 165 | APEKPEEDASPEELQRYQVSLRHYYNWLTRQRY | 169 |
| 166 | APEKPPADASPEELQRYYVELRHYYNWLTRQRY | 170 |
| 167 | APEKPEEDASPEELQQYEVSLRHYYNWLTRQRY | 171 |
| 168 | APEKPEEDAEPEELQRYYVSLRHYYNWITRQRY | 172 |
| 169 | APEKPEEEASPEELQRYYVALRHYYNWLTRQRY | 173 |
| 170 | APEKPEEEASPEELEQYYVSLRHYYNWLTRQRY | 174 |
| 171 | APEKPEEDATPEEIQRYYVSLRHYYNWLTRQRY | 175 |
| 172 | APEKPEEDAEPEELQRYYVALRHYYNWLTRQRY | 176 |
| 173 | APAKPEEDASPEELQRYYVELRHYYNWLTRQRY | 177 |
| 174 | APAKPEEDAEPEELQRYYVSLRHYYNWLTRQRY | 178 |
| 175 | APEKPEEDAAPEELQRYYVALRHYYNWLTRQRY | 179 |
| 176 | APEKPEEDASEEELQRYYVSLRHYYNWLTRQRY | 180 |
| 177 | APEKPEEDSSPEELQRYYVELRHYYNWLTRQRY | 181 |
| 178 | APEKPEEDATPEETERYYVSLRHYYNWLTRQRY | 182 |
| 179 | APAKPEEDASPAELQRYYVSLRHYYNWLTRQRY | 183 |
| 180 | APEKPEEDASPEETQQYYVSLRHYYNWLTRQRY | 184 |
| 181 | APEKPEADASPEELQRYYVALRHYYNWLTRQRY | 185 |
| 182 | APEKPEEDGSPEELQRYYVELRHYYNWLTRQRY | 186 |
| 183 | APEKPPEDASPEELQRYYVSLRHYYNWLTRQRY | 187 |
| 184 | APEKPEEAASPEELQRYYVTLRHYYNWLTRQRY | 188 |
| 185 | APEKPEEEASPEELQRYYVELRHYYNWLTRQRY | 189 |
| 186 | APEKPEEDASAEELQRYYVELRHYYNWLTRQRY | 190 |
| 187 | APEKPEEDASPEELQRYYVSLRHQYNWLTRQRY | 191 |
| 188 | APEKPEEDAEPEELQRYYVSLRHYYNWLTRQRY | 192 |
| 189 | APEKPEEDASEAELQRYYVSLRHYYNWLTRQRY | 193 |
| 190 | APEKPEEEAPPEELQRYYVSLRHYYNWLTRQRY | 194 |
| 191 | APEKPEEDASPEELQRYYISLRHYYNWLTRQRY | 195 |
| 192 | APEKPEEDAAPAELQRYYVSLRHYYNWLTRQRY | 196 |
| 193 | APEKPEEDASEEEIQRYYVSLRHYYNWLTRQRY | 197 |
| 194 | AHypEKPEEDASPEELQRYYVSLRHYYNWTLRQRY | 198 |
| 195 | APEKPEEDASPEALQRYYVELRHYYNWLTRQRY | 199 |
| 196 | APAKPPEDAEPEELQRYYVSLRHYYNWLTRQRY | 200 |
| 197 | APAKPEADASPEELQRYYVELRHYYNWLTRQRY | 201 |
| 198 | APEKPEEDAEPEALQRYYVSLRHYYNWLTRQRY | 202 |
| 199 | APEKPEEDAEPEETQRYYVSLRHYYNWLTRQRY | 203 |
| 200 | APEKHypEEDASPEELQRYYVSLRHYYNWLTRQRY | 204 |
| 201 | APEKPQEDAEPEELQEYYVSLRHYYNWLTRQRY | 205 |
| 202 | APEKPEEDAAPEELQKYYVSLRHYYNWLTRQRY | 206 |
| 203 | APAKPEEDASPEELQRYYVSLRHYYNWLTRQRY | 207 |
| 204 | APEKPEEEASPEEVQRYYVSLRHYYNWLTRQRY | 208 |
| 205 | APEKPEEEASPEELQKYYVSLRHYYNWLTRQRY | 209 |
| 206 | APEKPEEDASPEELEQYEVSLRHYYNWLTRQRY | 210 |
| 207 | APAKPEADAEPEELQRYYVSLRHYYNWLTRQRY | 211 |
| 208 | APEKPEEDASEEETQRYYVSLRHYYNWLTRQRY | 212 |
| 209 | APEKPEEDSEPEELQRYYVSLRHYYNWLTRQRY | 213 |
| 210 | APEKIEEEASPEELQRYYVSLRHYYNWLTRQRY | 214 |
| 211 | APEKPEEDATAEELQRYYVSLRHYYNWLTRQRY | 215 |
| 212 | APEKPEEDTAPEELQRYYVSLRHYYNWLTRQRY | 216 |
| 213 | APEKPEEETSPEELQRYYVSLRHYYNWLTRQRY | 217 |
| 214 | APEKPEEDASEEELQRYYVALRHYYNWLTRQRY | 218 |
| 215 | APEKPEEDASPAELQRYYVELRHYYNWLTRQRY | 219 |
| 216 | APAKPEEDASPEELEQYYVSLRHYYNWLTRQRY | 220 |
| 217 | APEKPEEDASPAELQRYYVALRHYYNWLTRQRY | 221 |
| 218 | APEKPEEDSSPEELQRYYVALRHYYNWLTRQRY | 222 |
| 219 | APEKPEEEAAPEELQRYYVSLRHYYNWLTRQRY | 223 |
| 220 | APEKPEEAASAEELQRYYVSLRHYYNWLTRQRY | 224 |
| 221 | APEKPEEDAPEEELQRYYVSLRHYYNWLTRQRY | 225 |
| 222 | APEKPEEDASAEELQQYYVSLRHYYNWLTRQRY | 226 |
| 223 | APEKPEEAAEPEELQRYYVSLRHYYNWLTRQRY | 227 |
| 224 | APEKPGEEASPEELEQYYVSLRHYYNWLTRQRY | 228 |
| 225 | APEKPGEDAEPEELEQYYVSLRHYYNWLTRQRY | 229 |
| 226 | APEKPEEAASPEETQRYYVSLRHYYNWLTRQRY | 230 |
| 227 | APEKAEEDASPEELEQYYVSLRHYYNWLTRQRY | 231 |
| 228 | APEKPEEDASPEELQQYYVSLRAYYNWLTRQRY | 232 |
| 229 | APEKPEEDASPEELQRYYVSARHYYNWLTRQRY | 233 |
| 230 | APEKPEEDASPEEIQRYYVALRHYYNWLTRQRY | 234 |
| 231 | APEKPEEDATPEELQKYYVSLRHYYNWLTRQRY | 235 |
| 232 | APAKPPEDASPEELQRYYVELRHYYNWLTRQRY | 236 |
| 233 | APEKPEEAASPEELQQYYVSLRHYYNWLTRQRY | 237 |
| 234 | APAKPEEEASPEELQRYYVSLRHYYNWLTRQRY | 238 |
| 235 | APEKPEEDASHypEELQRYYVSLRHYYNWLTRQRY | 239 |
| 236 | APEKPEEEAPAEELQRYYVSLRHYYNWLTRQRY | 240 |
| 237 | APEKPEEDASPEELQRYYVEVRHYYNWLTRQRY | 241 |
| 238 | APEKTEEEASPEELQRYYVSLRHYYNWLTRQRY | 242 |
| 239 | APEKPEEDASEEELQRYEVSLRHYYNWLTRQRY | 243 |
| 240 | APEKPEEDASPEALQRYYVSLRHYYNWLTRQRY | 244 |
| 241 | APEKPEEEASPEEIQRYYVSLRHYYNWLTRQRY | 245 |
| 242 | APEKPEADASAEELQRYYVSLRHYYNWLTRQRY | 246 |
| 243 | APEKPEEEASPEELQQYYVSLRHYYNWLTRQRY | 247 |
| 244 | APEKPEEDAEPAELQRYYVSLRHYYNWLTRQRY | 248 |

and wherein a half-life extending group is attached to the epsilon amino group of the lysine at position 7 or of a lysine at positions 6, 10, 11, 14, 17, 21 or 22, or to the carboxylic acid group of the side chain of an aspartate or a glutamate at position 14.

In an embodiment of the present invention, the PYY analogue is a compound having the formula:

R¹—Z—R²
wherein R¹ and R² are as defined above;
Z is an amino acid sequence of selected from Table 1; and
wherein a half-life extending group is attached to the epsilon amino group of the lysine at position 7 and consists of a lipophilic substituent X and a linker U,
wherein the linker U is attached to the amino acid side chain and X is attached to U, and the linker U consists of one, two or three sub-moieties (U1, U2, U3).

In some embodiments, the lipophilic substituent, X is selected from the group consisting of 15-carboxy-pentadecanoyl, 17-carboxy-heptadecanoyl (C18DA) and 19-carboxy-nonadecanoyl.

In some embodiments the linker U consists of one, two or three sub-moieties (U¹, U², U³) independently selected from the group consisting of Gly, Glu, γ-Glu, ε-Lys, Ser and OEG, or independently selected from the group consisting of γ-Glu and OEG.

In certain embodiments, the linker U is selected from the group consisting of γ-Glu, γ-Glu-γ-Glu, γ-Glu-OEG, OEG-OEG, γ-Glu-γ-Glu-OEG, γ-Glu-OEG-OEG.

In specific embodiments, the half-life extending group is C18DA-γ-Glu-OEG-OEG-, i.e.

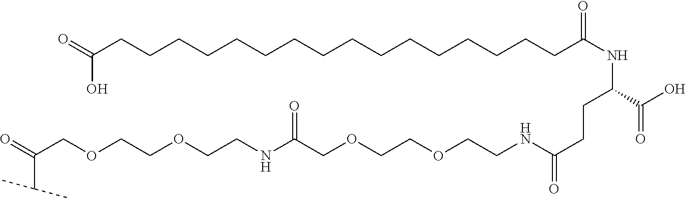

In some embodiments of the present invention, the PYY analogue is a compound selected from the group consisting of compound 1 to compound 244 as defined below.

In some embodiments, the PYY analogue has a maximum of 15 amino acid modifications as compared to hPYY(3-36).

In some embodiments, the PYY analogue has a maximum of 14 amino acid modifications as compared to hPYY(3-36).

In some embodiments, the PYY analogue has a maximum of 13 amino acid modifications as compared to hPYY(3-36).

In some embodiments, the PYY analogue has a maximum of 12 amino acid modifications as compared to hPYY(3-36).

In some embodiments, the PYY analogue has a maximum of 11 amino acid modifications as compared to hPYY(3-36).

In some embodiments, the PYY analogue has between 7 and 15 (i.e. 7, 8, 9, 10, 11, 12, 13, 14 or 15) amino acid modifications as compared to hPYY(3-36).

In some embodiments, the PYY analogue has between 8 and 14 (i.e. 8, 9, 10, 11, 12, 13 or 14) amino acid modifications as compared to hPYY(3-36).

In some embodiments, the PYY analogue has between 5 and 13 (i.e. 5, 6, 7, 8, 9, 10, 11, 12 or 13) amino acid modifications as compared to hPYY(3-36).

In some embodiments, the PYY analogue has between 7 and 13 (i.e. 7, 8, 9, 10, 11, 12 or 13) amino acid modifications as compared to hPYY(3-36).

In some embodiments, the PYY analogue has between 8 and 13 (i.e. 8, 9, 10, 11, 12 or 13) amino acid modifications as compared to hPYY(3-36).

In some embodiments, the PYY analogue has between 9 and 13 (i.e. 9, 10, 11, 12 or 13) amino acid modifications as compared to hPYY(3-36).

In some embodiments, the PYY analogue has between 10 and 13 (i.e. 10, 11, 12 or 13) amino acid modifications as compared to hPYY(3-36).

In some embodiments, the PYY analogue has between 8 and 12 (i.e. 8, 9, 10, 11 or 12) amino acid modifications as compared to hPYY(3-36).

In some embodiments, the PYY analogue has between 9 and 12 (i.e. 9, 10, 11 or 12) amino acid modifications as compared to hPYY(3-36).

In some embodiments, the PYY analogue has between 10 and 12 (i.e. 10, 11 or 12) amino acid modifications as compared to hPYY(3-36).

In some embodiments, the PYY analogue has between 9 and 11 (i.e. 9, 10 or 11) amino acid modifications as compared to hPYY(3-36).

In some embodiments, the PYY analogue has between 11 and 12 (i.e. 11 or 12) amino acid modifications as compared to hPYY(3-36).

In some embodiments, the PYY analogue or compound of the above-mentioned embodiments is in the form of a salt, preferably in the form of a pharmaceutically acceptable salt.

The PYY analogues of the invention are able to bind to the human NPY2 receptor (hNPY2-R).

Binding to biological receptors can be measured by appropriate assays known in the art. For instance, binding of PYY analogues to the NPY2 receptor can be evaluated by radio-ligand binding competition assays, e.g. as described in Example 1, below.

In some embodiments of compounds of the present invention, the binding affinity (Ki) towards hNPY2 receptor is below 100 nM (e.g. 0.01 to 100 nM).

In some embodiments of compounds of the present invention, the binding affinity (Ki) towards hNPY2 receptor is below 50 nM (e.g. 0.01 to 50 nM).

In some embodiments of compounds of the present invention, the binding affinity (Ki) towards hNPY2 receptor is below 10 nM (e.g. 0.01 to 10 nM).

In some embodiments of compounds of the present invention, the binding affinity (Ki) towards hNPY2 receptor is below 5 nM (e.g. 0.01 to 5 nM).

In some embodiments of compounds of the present invention, the binding affinity (Ki) towards hNPY2 receptor is below 2 nM (e.g. 0.01 to 2 nM).

The PYY analogues of the invention activate the human NPY2 receptor, i.e. they are NPY2 agonists.

In general, it is preferred to use a biological assay which measures intracellular signalling caused by binding of the compound to the relevant receptor. Activation of the NPY2 receptor by compounds of the invention (which behave as agonists of the receptor) reduces cAMP concentrations effecting intracellular signalling pathways. Thus, reduction of cAMP or any other suitable parameter in cells expressing the receptor can be used to monitor agonist activity towards the receptor. The skilled person will be aware of suitable assay formats, and examples are provided below.

EC₅₀ values may be used as a numerical measure of agonist potency at a given receptor. An EC₅₀ value is a measure of the concentration of a compound required to achieve half of that compound's maximal activity in a particular assay, e.g. in the assay as described in Example 2, below.

In some embodiments of compounds of the present invention, the EC₅₀ towards hNPY2 receptor is below 100 nM (e.g. 0.001 to 100 nM).

In some embodiments of compounds of the present invention, the EC₅₀ towards hNPY2 receptor is below 50 nM (e.g. 0.001 to 50 nM).

In some embodiments of compounds of the present invention, the EC₅₀ towards hNPY2 receptor is below 10 nM (e.g. 0.001 to 10 nM).

In some embodiments of compounds of the present invention, the EC₅₀ towards hNPY2 receptor is below 5 nM (e.g. 0.001 to 5 nM).

In some embodiments of compounds of the present invention, the EC₅₀ towards hNPY2 receptor is below 2 nM (e.g. 0.001 to 2 nM).

As mentioned above the PYY analogues or compounds of the present invention are generally soluble around pH 7 and 6. There are several techniques known to the skilled person in the art how to determine solubility. One such experiment is described below under Example 3. If specific solubility in mg/ml is provided herein, it is referred to the solubility determination as in Example 3.

In some embodiments, the solubility of the PYY analogues or compounds of the invention is greater than 1.0 mg/ml around pH 6 (e.g. at pH 6.1±0.2).

In some embodiments, the solubility of the PYY analogues or compounds of the invention is greater than 3.0 mg/ml around pH 6 (e.g. at pH 6.1±0.2).

In some embodiments, the solubility of the PYY analogues or compounds of the invention is greater than 5.0 mg/ml around pH 6 (e.g. at pH 6.1±0.2). In some embodiments, the solubility of the PYY analogues or compounds of the invention is equal to or greater than 7.0 mg/ml around pH 6 (e.g. at pH 6.1±0.2).

In some embodiments, the solubility of the PYY analogues or compounds of the invention is equal to or greater than 8.0 mg/ml around pH 6 (e.g. at pH 6.1±0.2).

In some embodiments, the solubility of the PYY analogues or compounds of the invention is greater than 1.0 mg/ml around pH 7 (e.g. at pH 6.8±0.2).

In some embodiments, the solubility of the PYY analogues or compounds of the invention is greater than 5.0 mg/ml around pH 7 (e.g. at pH 6.8±0.2).

In some embodiments, the solubility of the PYY analogues or compounds of the invention is is greater than 7.0 mg/ml around pH 7 (e.g. at pH 6.8±0.2).

In some embodiments, the solubility of the PYY analogues or compounds of the invention is equal to or greater than 8.0 mg/ml around pH 7 (e.g. at pH 6.8±0.2).

In some embodiments, the solubility of the PYY analogues or compounds of the invention is greater than 1.0 mg/ml around pH 6 (e.g. at pH 6.1±0.2) and around pH 7 (e.g. at pH 6.8±0.2).

In some embodiments, the solubility of the PYY analogues or compounds of the invention is greater than 3.0 mg/ml around pH 6 (e.g. at pH 6.1±0.2) and around pH 7 (e.g. at pH 6.8±0.2).

In some embodiments, the solubility of the PYY analogues or compounds of the invention is greater than 5.0 mg/ml around pH 6 (e.g. at pH 6.1±0.2) and around pH 7 (e.g. at pH 6.8±0.2).

In some embodiments, the solubility of the PYY analogues or compounds of the invention is greater than 6.0 mg/ml around pH 6 (e.g. at pH 6.1±0.2) and around pH 7 (e.g. at pH 6.8±0.2).

In some embodiments the PYY analogues or compounds of the invention have favourable pharmacokinetic properties. In this regard, in some embodiments of the invention, the in-vivo half-life of the PYY analogues or compounds is at least 3 hours in the mouse (NMRI mice, see measurement described in Example 5). In some embodiments, the in-vivo half-life is at least 5 hours in the mouse. In some embodiments, the in-vivo half-life is at least 7 hours in the mouse. In some embodiments, the in-vivo half-life is at least 10 hours in the mouse.

The invention further provides a composition comprising a PYY analogue as described above. The composition may be a pharmaceutical composition, and may comprise a pharmaceutically acceptable carrier, excipient or vehicle.

The invention further provides a method for the synthesis of a PYY analogue as described above. The method may comprise the steps of synthesising the peptide by solid-phase or liquid-phase methodology, and optionally isolating and/or purifying the final product.

### Method of Treatment

The present invention is directed to PYY analogues or a compound according to the above-mentioned embodiments, which are useful in the prevention and/or treatment of a disease and/or condition associated with or modulated by NPY2 receptor activity, including but not limited to the treatment and/or prevention of obesity and various obesity-related conditions, diseases, or co-morbidities, such as type 2 diabetes and NASH (non-alcoholic steatohepatitis).

The compounds described herein find use, inter alia, in preventing weight gain or promoting weight loss. By “preventing” is meant inhibiting or reducing when compared to the absence of treatment, and is not necessarily meant to imply complete cessation of weight gain. The peptides may cause a decrease in food intake and/or increased energy expenditure, and may have a beneficial effect on glucose control and/or on circulating cholesterol levels, being capable of lowering circulating LDL levels and increasing HDL/LDL ratio. Thus, the compounds of the invention can be used for direct or indirect therapy of any condition caused or characterised by excess body weight, such as the treatment and/or prevention of obesity, morbid obesity, obesity linked inflammation, obesity linked gallbladder disease, and obesity related sleep apnea. They may also be used for the prevention of conditions or treatment of obesity associated co-comorbidities caused or characterised by inadequate glucose control or dyslipidaemia (e.g. elevated LDL levels or reduced HDL/LDL ratio), Type 2 diabetes, metabolic syndrome, hypertension, atherogenic dyslipidemia, and cardiovascular diseases such as atherosclerosis, coronary heart disease, peripheral artery disease, stroke or microvascular disease, and cancer. Their effects in these conditions may be as a result of or associated with their effect on body weight, or may be independent thereof.

As mentioned above the PYY analogues or compounds according to the above mentioned embodiments are useful in the reduction of food intake, promotion of weight loss, and inhibition or reduction of weight gain. As a result, they may be used for treatment of a variety of conditions, diseases, or disorders in a subject, including, but not limited to, obesity and various obesity-related conditions, diseases, or co-morbidities, such as type 2 diabetes, hypertension, dyslipidemia, sleep apnea, cardiovascular disease, hepatic steatosis, non-alcoholic fatty liver disease (NAFLD), non-alcoholic steatohepatitis (NASH) and cancer. The subject may be affected by obesity accompanied by at least one weight-related comorbid condition, such as type 2 diabetes, hypertension, dyslipidemia, sleep apnea, cardiovascular disease, hepatic steatosis, NAFLD and NASH. It will be understood that the PYY analogues may thus be administered to subjects affected by conditions characterised by inadequate control of appetite or otherwise over-feeding, such as binge-eating disorder and Prader-Willi syndrome. It will be clear that the analogues can be used for treatment of combinations of the conditions described.

Thus, the invention provides a PYY analogue for use in a method of medical treatment, e.g. for treating, inhibiting or reducing weight gain, promoting weight loss and/or reducing excess body weight. Treatment may be achieved, for example, by control of appetite, feeding, food intake, caloric intake and/or energy expenditure.

The invention also provides a PYY analogue of the invention for use in a method of treating obesity as well as associated diseases, disorders and health conditions, including, but not limited to, morbid obesity, obesity prior to surgery, obesity-linked inflammation, obesity-linked gallbladder disease and obesity related sleep apnea and respiratory problems, degeneration of cartilage, osteoarthritis, and reproductive health complications of obesity or overweight such as infertility. The subject may be affected by obesity accompanied by at least one weight-related co-morbidity, such as type 2 diabetes, hypertension, dyslipidemia, sleep apnea, cardiovascular disease, cancer, hepatic steatosis, NAFLD and NASH.

The invention also provides a PYY analogue of the invention for use in a method of prevention or treatment of conditions mentioned above.

Accordingly, the present invention relates to a PYY analogue or a compound according to the above-mentioned embodiments for use as a medicament.

Furthermore, the present invention relates to the use of a PYY analogue or a compound according to the above-mentioned embodiments for the treatment and/or prevention of a disease and/or condition associated with or modulated by NPY2 receptor activation. Furthermore, the present invention relates to the use of a PYY analogue or a compound according to the above mentioned embodiments for the treatment and/or prevention of obesity and various obesity-related conditions, diseases, or co-morbidities, such as type 2 is diabetes and NASH (non-alcoholic steatohepatitis) and others as mentioned above.

In a further aspect the present invention relates to the use of a PYY analogue or a compound according to the above mentioned embodiments for the preparation of a medicament for the treatment and/or prevention of above-mentioned diseases and conditions.

In a further aspect the present invention relates to methods for the treatment or prevention of above mentioned diseases and conditions, which method comprises the administration of an effective amount of a PYY analogue or a compound according to the above-mentioned embodiments to a human being.

The dose range of the compounds of general formula I applicable per week is usually from 0.01 to 100 mg for humans (subcutaneous administration).

The actual pharmaceutically effective amount or therapeutic dosage will usually depend on factors known by those skilled in the art such as age and weight of the patient, route of administration and severity of disease. In any case, the compounds will be administered at dosages and in a manner, which allows a pharmaceutically effective amount to be delivered based upon patient's unique condition.

### Combination Therapy

A PYY analogue of the invention may be administered as part of a combination therapy together with another active agent for the treatment of the disease or disorder in question, e.g. an anti-obesity agent, an anti-diabetic agent, an agent for treatment of metabolic syndrome, an anti-dyslipidemia agent, an anti-hypertensive agent, a proton pump inhibitor, or an anti-inflammatory agent. In such cases, the two active agents may be given together or separately, e.g. as constituents in the same pharmaceutical composition or formulation, or as separate formulations.

Thus, a peptide of the invention may be used in combination with an anti-obesity agent of known type. The anti-obesity agent may be a GIP or GLP-1 receptor agonist (including is GLP-1 or a GLP-1 analogue, exendin-4 or an exendin-4 analogue, any other GLP-1 receptor agonist including Liraglutide (Saxenda™), Semaglutide, Dulaglutide, Albiglutide, MK-8521, or a glucagon-GLP-1 dual agonist (e.g. HM-12525, SAR-425899, MEDI-0382, NN-9277 or as described in WO2008/101017, WO2008/152403, WO2010/070252, WO2010/070253, WO2010/070255, WO2010/070251, WO2011/006497, WO2011/160630, WO2011/160633, WO2013/092703, WO2014/041195, WO2015/055801, WO2015/055802, WO2016/166289), oxyntomodulin or an oxyntomodulin analog (e.g. TT-401) or a GLP-1/GIP dual agonist (e.g. Tirzepatide or as described in WO2013/164483), or a GLP-1/GIP/glucagon triple agonists (e.g. NN-9423 or as described in WO2015/067716, WO2016/198624, WO2017/116204, WO2017/116205, WO2018/100134, WO2018/100135).

The anti-obesity agent may be amylin or an amylin analogue, e.g. pramlintide, NN-9838, or an amylin (or calcitonin) analogue disclosed in WO2012/168430, WO2012/168431, WO2012/168432, WO2015/040182, WO2015/071229, WO2016/146739, WO2018/046719, or WO2018/172390.

Alternatively, the anti-obesity agent may be Orlistat™, Sibutramine™, phentermine, a melanin concentrating hormone receptor 1 antagonist, CCK, leptin analogue, a GOAT inhibitor, a ghrelin-receptor antagonist, a further neuropeptide Y (NPY) analogue, a NPY4 receptor agonist, a NPY5 receptor antagonist, a cannabinoid receptor 1 antagonist, a beta-3 agonist, a lipase inhibitor, Human prolslet Peptide (HIP), a melanocortin receptor 4 agonist, as well as analogues thereof.

Moreover, a peptide of the invention may have some benefit if administered in combination with an anti-diabetic agent of known type, including, but not limited to, metformin, a sulfonylurea, a glinide, a DPP-IV inhibitor, a glitazone, a GLP-1 receptor agonist (including GLP-1 or a GLP-1 analogue, an exendin-4 or an exendin-4 analogue, any other GLP-1 receptor agonist including Liraglutide (Victoza™), Semaglutide, Dulaglutide, Albiglutide, MK-8521, or a glucagon-GLP-1 dual agonist (e.g. HM-12525, SAR-425899, MEDI-0382, NN-9277 or as described in WO2008/101017, WO2008/152403, WO2010/070252, WO2010/070253, WO2010/070255, WO2010/070251, WO2011/006497, WO2011/160630, WO2011/160633, WO2013/092703, WO2014/041195, WO2015/055801, WO2015/055802, WO2016/166289), oxyntomodulin, or an oxyntomodulin analog (e.g. TT-401), or a SGLT2 inhibitor (i.e. an inhibitor of is sodium-glucose transport, e.g. a gliflozin such as empagliflozin, canagliflozin, dapagliflozin or ipragliflozin), a GPR40 agonist (FFAR1/FFA1 agonist, e.g. fasiglifam), or an insulin or an insulin analogue. Examples of appropriate insulin analogues include, but are not limited to, Lantus™, Novorapid™, Humalog™, Novomix™ Actraphane™ HM, Levemir™ Degludec™ and Apidra™. Other relevant anti-diabetic agents in this connection include GLP-1 receptor agonists, such as exenatide (Byetta™ and Bydureon™ exendin-4) and Byetta LAR™, and lixisenatide (Lyxumia™)

According to more specific embodiments, the PYY analogue of the present invention is administered as part of a combination therapy together with
a GLP-1 receptor agonist selected from the group consisting of Liraglutide, Semaglutide, Dulaglutide and Albiglutide or a glucagon-GLP-1 dual agonist described in WO2011/006497, WO2014/041195, WO2015/055801, WO2015/055802, WO2016/166289 or
an amylin receptor agonist selected from the group consisting of pramlintide or an amylin analogue disclosed in WO2012/168430, WO2012/168431, WO2012/168432, WO2015/040182, WO2016/146739, or WO2018/046719.

A peptide of the invention may further be used in combination with medications targeting cardiovascular diseases treating hypertension, dyslipidemia, inflammation and platelet function. The medication treating hypertension can be selected from the group including, but not limited to, an angiotensin-converting enzyme inhibitor, an angiotensin II receptor blocker, a diuretic, a beta-blocker or a calcium channel blocker.

A peptide of the invention may still further be used in combination with an antidyslipidemia agent of known type, including, but not limited to, a statin, a fibrate, a niacin, a PSCK9 (Proprotein convertase subtilisin/kexin type 9) inhibitor, or a cholesterol absorption inhibitor.

A peptide of the invention may also be used in combination with a proton pump inhibitor (i.e. a pharmaceutical agent possessing pharmacological activity as an inhibitor of H⁺/K⁺-ATPase) of known type, including, but not limited to, an agent of the benzimidazole derivative type or of the imidazopyridine derivative type, such as Omeprazole™, Lansoprazole™, Dexlansoprazole™, Esomeprazole™, Pantoprazole™, Rabeprazole™, Zolpidem™, Alpidem™, Saripidem™ or Necopidem™.

In addition, with regard to anti-inflammatory treatment, a peptide of the invention may be beneficial if administered in combination with an anti-inflammatory agent of known type, including, but not limited to:
steroids and corticosteroids, such as beclomethasone, methylprednisolone, betamethasone, prednisone, dexamethasone, and hydrocortisone;
non-steroidal anti-inflammatory agents (NSAIDs), such as propionic acid derivatives (e.g. alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid and tioxaprofen); acetic acid derivatives (e.g. indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin and zomepirac); fenamic acid derivatives (e.g. flufenamic acid, meclofenamic acid, mefenamic acid, niflumic acid and tolfenamic acid); biphenylcarboxylic acid derivatives (e.g. diflunisal and flufenisal); oxicams (e.g. isoxicam, piroxicam, sudoxicam and tenoxicam); salicylates (e.g. acetylsalicylic acid and sulfasalazine); and pyrazolones (e.g. apazone, bezpiperylon, feprazone, mofebutazone, oxyphenbutazone and phenylbutazone);
COX II inhibitors, such as rofecoxib and celecoxib; preparations of interferon beta (e.g. interferon beta-1a or interferon beta-1b);
and certain other compounds, such as 5-aminosalicylic acid and prodrugs and pharmaceutically acceptable salts thereof.

### Preparation

### General Procedure for Solid Phase Synthesis of Peptides

All peptides were synthesized by standard Fmoc-based solid phase peptide chemistry on a Tentagel S RAM resin (loading 0.23-0.25 mmol/g, bead size 90 μm) supplied by Iris Biotech GmbH or Rapp Polymere GmbH.

The following protected amino acids were used: Fmoc-Ala-OH, Fmoc-Arg(Pbf)-OH, Fmoc-Asn(Trt)-OH, Fmoc-Asp(tBu)-OH, Fmoc-Gln(Trt)-OH, Fmoc-Glu(tBu)-OH, FmocGlu-OtBu, Fmoc-Gly-OH, Fmoc-His(Trt)-OH, Fmoc-Ile-OH, Fmoc-Leu-OH, Fmoc-Lys(Boc)-OH, Fmoc-Lys(Dde)-OH, Fmoc-Phe-OH, Fmoc-Pro-OH, Fmoc-Ser(tBu)-OH, Fmoc-Thr(tBu)-OH, Fmoc-Trp(Boc)-OH, Fmoc-Tyr(tBu)-OH, Fmoc-Val-OH. The L-form of the amino acid building blocks was utilized if not specified otherwise. The modular half-life extending group was built up by solid-phase peptide synthesis (SPPS) using protected building blocks such as, but not limited to, 18-(tert-butoxy)-18-oxooctadecanoic acid (C18DA(tBu)), 2-[2-[2-[[2-[2-[2-(9H-fluoren-9-ylmethoxycarbonylamino)ethoxy]ethoxy]acetyl]amino]ethoxy]ethoxy]acetic acid (Fmoc-OEG-OEG-OH), 2-[2-[2-(9H-fluoren-9-ylmethoxycarbonylamino)ethoxy]ethoxy]acetic acid (Fmoc-OEG-OH) and Fmoc-Glu-OtBu.

The amino acids, Fmoc-Glu-OtBu, Oxyma and DIC, were purchased from standard suppliers, e.g. Bachem, Novabiochem, ABCR, Iris Biotech GmbH, Sigma-Aldrich. 18-(Tert-butoxy)-18-oxooctadecanoic acid (C18DA(tBu)) was supplied by Cool Pharm Ltd. or AstraTech, 2-[2-[2-[[2-[2-[2-(9H-Fluoren-9-ylmethoxycarbonylamino)ethoxy]ethoxy]acetyl]amino]ethoxy]ethoxy]acetic acid (Fmoc-OEG-OEG-OH) was supplied by ABCR GmbH & CO. KG or Iris Biotech GmbH, 2-[2-[2-(9H-fluoren-9-ylmethoxycarbonylamino)ethoxy]ethoxy]acetic acid (Fmoc-OEG-OH) was supplied by Combi Blocks Inc., Iris Biotech GmbH or Hangzhou APIChem Technology Co., Ltd. 3-Methylbutanoic acid was supplied by Sigma-Aldrich GmbH.

Assembly of peptides started from the C-terminus by stepwise chain elongation towards the N-terminus according to the respective sequences until the N-terminal capping group was reached. Deprotection of the side chain of the branching amino acid, e.g. Lys(Dde), was followed by assembly of the half-life extending group.

The PYY analogues were obtained as TFA salts from the cleavage/deprotection or from the HPLC purification. The trifluoroacetate can be exchanged by common procedures, such as resin-ion exchange procedures, e.g. as disclosed in Roux, St. et al. J. Pept. Sci. 2008; 14: 354-359.

### Synthesis Method 1 (S01)

Peptides were synthesized by microwave-assisted solid-phase peptide synthesis (SPPS) on a CEM Liberty Blue Peptide Synthesizer at 0.25 mmol scale on Tentagel S RAM resin using the Fmoc strategy.

Standard coupling of amino acids was performed with 4 eq of suitably protected amino acid in DMF (0.2 mol/l, 5 ml), 4 eq of Oxyma in DMF (1 mol/l, 1 ml) and 8 eq DIC in DMF (1 mol/l, 2 ml) at 90 °C for 4 min. Fmoc-Arg(Pbf)-OH was coupled 2 times at 90 °C for 4 min, Fmoc-His(Trt)-OH was coupled 2 times at 50 °C for 12 min and Fmoc-Glu-OtBu was coupled 4 times at 50 °C for 12 min. Fmoc-OEG-OH, Fmoc-OEG-OEG-OH and C18DA(tBu) were coupled 2 times for 4 min at 90 °C. Capping of the N-terminus was achieved by coupling 3-methylbutanoic acid 3 times at 90 °C for 4 min.

N^{α} Fmoc deprotection was performed with 20% piperidine/DMF (10 ml) for 1 min at 90 °C. Deprotection of the Lys(Dde)-group was carried out 2 times with 5% hydrazine hydrate in DMF (10 ml) for 3 min at 90 °C.

Raw products were washed on resin with DCM and dried prior to cleavage. Cleavage from resin and deprotection was performed with a mixture of 95% TFA/water (10 ml) and triisopropylsilane (250 μl) for 40 min at 42 °C. Crude peptides were precipitated with cold diethyl ether, dissolved in 50% acetonitrile/water and purified by preparative HPLC (P01).

### Synthesis Method 2 (S02)

Peptides were synthesized by SPPS on a MultiSynTech SYRO II at 0.2 mmol scale.

Standard coupling of amino acids was achieved by using 4 eq of suitably protected amino acids dissolved in 0.5 mol/1 Oxyma-DMF solution (0.5 mol/l, 1.6 ml) and 4.5 eq DIC (0.5 mol/1, 1.8 ml) in DMF. Fmoc-Phe-OH was dissolved in 0.5 mol/1 Oxyma-NMP and 4 eq were used for coupling (0.5 mol/1, 1.6 ml).

Coupling time of the first 15 amino acids starting from the C-terminus was 2 h at RT. Subsequent couplings were realized by double coupling (2×2 h at RT). Capping of the N-terminus was achieved by coupling with 3-methylbutanoic acid (2×2 h at RT). Deprotection of Lys(Dde)-group was carried out selectively using 5% hydrazine in DMF (5×5 min at RT, 4 ml) followed by coupling of 4 eq Fmoc-OEG-OEG-OH (3×2 h at RT), 4 eq FmocGlu-OtBu and 4 eq C18DA(tBu) (2×2 h at RT).

N^{α} Fmoc deprotections were performed using 40% piperidine in NMP (4 ml) for 3 min followed by 20% piperidine in NMP (4 ml) for 15 min at RT.

Peptides were cleaved from resin and side-chains deprotected by adding 15 ml 95:2:1:2 TFA:DODT:TES:water for 4 h at RT or 45 min at 45 °C. The peptides were precipitated with cold diethyl ether, dissolved in acetonitrile/water and purified by preparative HPLC(P02).

### Synthesis Method 3 (S03)

Peptides were synthesized using microwave-assisted SPPS on a Biotage Alstra Synthesizer at 0.2 mmol scale.

Standard coupling of amino acids was achieved by using 5 eq of suitably protected amino acid dissolved in 0.5 mol/1 Oxyma-DMF solution (0.5 mol/1, 2 ml) and 5 eq DIC in DMF (0.5 mol/1, 2 ml). Coupling of the first 15 amino acids starting from the C-terminus was achieved by heating for 5 min to 75 °C. Subsequent couplings were realized by double coupling (2×5 min at 75 °C).

N^{α} Fmoc deprotections were carried out using 20% piperidine in DMF for 30 sec followed by 20% piperidine for 3 min at 75 °C.

Special conditions were used for: coupling of Fmoc-His(Trt)-OH (2×12 min at 50 °C) and subsequent Fmoc-deprotection at RT (20% piperidine in DMF for 3 min followed by 20% piperidine in DMF for 10 min); coupling of Fmoc-Glu-OtBu (2×6 min at 75 °C) and Fmoc-Arg(Pbf)-OH (2×5 min at 75 °C); coupling of Fmoc-Asp(tBu)-OH and subsequent Fmoc-deprotection at RT (20% piperidine in DMF for 3 min followed by 20% piperidine in DMF for 10 min); Dde deprotection with 5% hydrazine in DMF (5×5 min at RT); coupling of 4 eq Fmoc-OEG-OEG-OH (3×10 min at 75 °C) and 2.5 eq C18DA(tBu) (2×10 min at 75 °C).

Cleavage from resin and side-chain deprotection was performed by adding 8 ml 95:2:1:2 TFA:DODT:TES:water for 4 hours at RT or 45 min at 45 °C. Crude peptides were precipitated with cold diethyl ether, dissolved in 50% acetonitrile/water and purified by preparative HPLC (P02).

### Purification Method 1 (P01)

Crude peptides were purified by reversed phase chromatography using an Agilent preparative HPLC-MS System with preparative pumps G1361A, a diode array detector G1315B, a mass-spectrometer G1956B and a fraction collector CTC PAL IFC. A Waters XSelect CSH Prep C18 column (130 Å, 5 μm, OBD, 30 mm×150 mm) served as stationary phase. The mobile phase was run with a gradient of buffer A (0.1% TFA in H₂O) and buffer B (0.1% TFA in ACN, gradient: 20-42% over 44 min) at a flow rate of 50 ml/min at 40 °C. The relevant fractions were pooled and lyophilized. The final product was characterized by analytical HPLC-MS (A01).

### Purification Method 2 (P02)

Crude peptides were purified by reversed phase HPLC using a Waters preparative HPLC with C8 column (Reprosil Gold 200 Å, 5 μm, 40 mm×250 mm), preparative pumps (waters 2545), UV/VIS detector (Waters 2489) and a Waters fraction collector III. The mobile phase was run with a gradient of buffer A (0.1% TFA in H₂O) and buffer B (0.1% TFA in ACN, gradient: 35-45% B over 10 min) at a flow rate of 50 ml/min at RT. Relevant fractions were analysed, pooled and lyophilized. The final product was characterized by analytical UPLC-MS (A02).

### Analytical Method 1 (A01)

Peptide purity and mass were determined by analytical HPLC-MS on a Kinetex C8 column (Phenomenex, 100 Å, 2.6 μm, 4.6 mm×150 mm) using a Waters Acquity HPLC System equipped with 3100 Mass Detector. Analysis was performed by gradient elution with buffer A (0.3% TFA in H₂O) and buffer B (0.24% TFA in ACN) at a temperature of 40 °C. Details of the gradient and flow rates are summarized in the table below. Retention times and masses were recorded.

| | | | % Sol | % Sol | | |
|---|---|---|---|---|---|---|
| | | Gradient/ | [Water | [ACN | | |
| | | Solvent | 0.3% | 0.24% | Flow | |
| | | Time | TFA | TFA | [ml/ | Temp |
| Method | A01 | [min] | (v/v)] | (v/v)] | min] | [° C.] |
| Device | Waters | 0.0 | 65.0 | 35.0 | 0.5 | 40.0 |
| description: | Acquity with | | | | | |
| | 3100 MS | | | | | |
| Column: | Kinetex | 15.0 | 45.0 | 55.0 | 0.5 | 40.0 |
| | C8_4.6 × | | | | | |
| | 150 mm_2.6 | | | | | |
| | μm | | | | | |
| Column | Phenomenex | 16.0 | 10.0 | 90.0 | 1.0 | 40.0 |
| producer: | | | | | | |
| | | 17.01 | 65.0 | 35.0 | 1.0 | 40.0 |
| | | 18.0 | 65.0 | 35.0 | 1.0 | 40.0 |

### Analytical Method 2 (A02)

Analytical UPLC-MS was performed on a Waters Acquity class H using a Waters Acquity UPLC C18 column (peptide CSH™ 130A, 1.7 m, 2.1 mm×100 mm) and a gradient flow of buffer C (0.3% TFA in H₂O) and buffer D (10% H₂O+0.3% TFA in ACN; 38-48% D over 14 min) connected to a SQ Detector 2 (ESI, Waters).

### LIST OF ABBREVIATIONS

ACN: acetonitrile
Boc: tert-butyloxycarbonyl
C18DA(tBu): 18-(tert-butoxy)-18-oxooctadecanoic acid
DPBS: Dulbecco's phosphate-buffered saline
DCM: dichloromethane
DIC: diisopropylcarbodiimide
DIPEA: diisopropylethylamine
Dde: (4,4-dimethyl-2,6-dioxocyclohex-1-ylidene)ethyl
DMF: N,N-dimethylformamid
DODT: 3,6-dioxa-1,8-octanedithiol
Fmoc: 9H-fluoren-9-ylmethoxycarbonyl
Fmoc-OEG-OH: 2-[2-[2-(9H-fluoren-9-ylmethoxycarbonylamino)ethoxy]ethoxy]acetic acid
Fmoc-OEG-OEG-OH: 2-[2-[2-[[2-[2-[2-(9H-fluoren-9-ylmethoxycarbonylamino)ethoxy]ethoxy]acetyl]amino]ethoxy]ethoxy]acetic acid
HTRF: homogeneous time resolved fluorescence
IBMX: 3-isobutyl-1-methylxanthine
iVal: 3-methylbutanoyl (isovalerianoyl)
MRT: mean residence time
NMP: 1-methyl-pyrrolidine-2-one
Oxyma: 2-cyano-2-(hydroxyimino)acetic acid ethyl ester
OEG: 2-[2-(2-aminoethoxy)ethoxy]acetic acid
Pbf: 2,2,4,6,7-pentamethyldihydrobenzofuran-5-sulfonyl
Rt: retention time
RT: room temperature
SPPS: solid-phase peptide synthesis
tBu: tert-butyl
Trt: trityl
TES: triethylsilane
TFA: trifluoroacetic acid

The following compounds were synthesised. All compounds were obtained as TFA salts:

### Compound 1

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,13Q,18Q,19E,22V,28Y,30W,31L]hPYY(4-36)

iVal-APEK(C18DA-gGlu-OEG1-OEG2-)
PEEDAQPEELQEYYVSLRHYYNWLTRQRY-NH2 (sequence
and structure below disclosed as SEQ ID NO: 5)

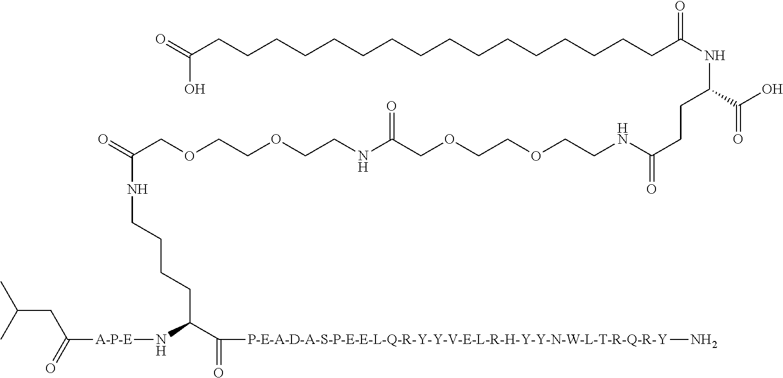

MW (calculated): 5001.6 Da

Synthesis and purification methods: S01; P01

LCMS: A01; Rt: 10.17 min; m/3: 1668.0 m/4: −m/5: −

### Compound 2

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,5Hyp,7K,9E,18Q,22V,28Y,30W,31L]hPYY(4-36)

(SEQ ID NO: 6)
iVal-AHypEK(C18DA-gGlu-OEG1-OEG2-)PEEDASPEELQRYYV
SLRHYYNWLTRQRY-NH2

MW (calculated): 5003.6 Da

Synthesis and purification methods: S01; P01

LCMS: A01; Rt: 9.29 min; m/3: 1668.0 m/4: − m/5: −

### Compound 3

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,15A,18Q,22V,28Y,30W,31L]hPYY(4-36)

(SEQ ID NO: 7)
iVal-APEK(C18DA-gGlu-OEG1-OEG2-)PEEEASPAELQRYYVSL
RHYYNWLTRQRY-NH2

Synthesis and purification methods: S01; P01

LCMS: A01; Rt: 9.86 min; m/3: − m/4: 1237.0 m/5: −

### Compound 4

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,18Q,22V,28Y,30W,31L,32Q]hPYY(4-36)

(SEQ ID NO: 8)
iVal-APEK(C18DA-gGlu-OEG1-OEG2-)PEEDASPEELQRYYVSL
RHYYNWLQRQRY-NH2

MW (calculated): 5014.6 Da

Synthesis and purification methods: S01; P01

LCMS: A01; Rt: 9.31 min; m/3: 1672.0 m/4: − m/5: −

### Compound 5

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,12V,18Q,22V,28Y,30W,31L]hPYY(4-36)

(SEQ ID NO: 9)
iVal-APEK(C18DA-gGlu-OEG1-OEG2-)PEEEVSPEELQRYYVSL
RHYYNWLTRQRY-NH2

MW (calculated): 5029.7 Da

Synthesis and purification methods: S01; P01

LCMS: A01; Rt: 10.02 min; m/3: − m/4: 1258.0 m/5: −

### Compound 6

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,18Q,22V,24A,28Y,30W,31L]hPYY(4-36)

(SEQ ID NO: 10)
iVal-APEK(C18DA-gGlu-OEG1-OEG2-)
PEEEASPEELQRYYVSARHYYNWLTRQRY-NH2

MW (calculated): 4959.6 Da

Synthesis and purification methods: S01; P01

LCMS: A01; Rt: 9.32 min; m/3: − m/4: 1241.0 m/5: −

### Compound 7

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,18Q,19A,22V,28Y,30W,31L]hPYY(4-36)

(SEQ ID NO: 11)
iVal-APEK(C18DA-gGlu-OEG1-OEG2-)
PEEDASPEELQAYYVSLRHYYNWLTRQRY-NH2

MW (calculated): 4902.5 Da

Synthesis and purification methods: S01; P01

LCMS: A01; Rt: 10.00 min; m/3: − m/4: 1226.0 m/5: −

### Compound 8

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,14E,18Q,19Q,22V,28Y,30W,31L]hPYY(4-36)

(SEQ ID NO: 12)
iVal-APEK(C18DA-gGlu-OEG1-OEG2-)
PEEDASEEELQQYYVSLRHYYNWLTRQRY-NH2

MW (calculated): 4991.6 Da

Synthesis and purification methods: S02; P02

LCMS: A02; Rt: 12.75 min; m/3: 1665.3 m/4: 1248.9 m/5: 999.2

### Compound 9

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,17I,18Q,22V,23E,28Y,30W,31L]hPYY(4-36)

(SEQ ID NO: 13)
iVal-APEK(C18DA-gGlu-OEG1-OEG2-)
PEEDASPEEIQRYYVELRHYYNWLTRQRY-NH2

MW (calculated): 5029.6 Da

Synthesis and purification methods: S01; P01

LCMS: A01; Rt: 9.60 min; m/3: 1677.0 m/4: − m/5: −

### Compound 10

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,18Q,22V,23E,24I,28Y,30W,31L]hPYY(4-36)

(SEQ ID NO: 14)
iVal-APEK(C18DA-gGlu-OEG1-OEG2-)
PEADASPEETQRYYVSLRHYYNWLTRQRY-NH2

MW (calculated): 4917.5 Da

Synthesis and purification methods: S01; P01

LCMS: A01; Rt: 9.21 min; m/3: − m/4: 1230.0 m/5: −

### Compound 11

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,17I,18Q,19Q,22V,28Y,30W,31L]hPYY(4-36)

(SEQ ID NO: 15)
iVal-APEK(C18DA-gGlu-OEG1-OEG2-)
PEEDASPEEIQQYYVSLRHYYNWLTRQRY-NH2

MW (calculated): 4959.6 Da

Synthesis and purification methods: S01; P01

LCMS: A01; Rt: 9.79 min; m/3: − m/4: 1241.0 m/5: −

### Compound 12

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,13P,14G,18Q,22V,28Y,30W,31L]hPYY(4-36)

(SEQ ID NO: 16)
iVal-APEK(C18DA-gGlu-OEG1-OEG2-)
PEEDAPGEELQRYYVSLRHYYNWLTRQRY-NH2

MW (calculated): 4957.6 Da

Synthesis and purification methods: S01; P01

LCMS: A01; Rt: 10.87 min; m/3: − m/4: 1240.0 m/5: −

### Compound 13

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,13A,18Q,19Q,22V,28Y,30W,31L]hPYY(4-36)

(SEQ ID NO: 17)
iVal-APEK(C18DA-gGlu-OEG1-OEG2-)
PEEDAAPEELQQYYVSLRHYYNWLTRQRY-NH2

MW (calculated): 4943.6 Da

Synthesis and purification methods: S01; P01

LCMS: A01; Rt: 10.70 min; m/3: − m/4: 1237.0 m/5: −

### Compound 14

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,13A,18Q,19Q,22V,28Y,30W,31L]hPYY(4-36)

iVal-APEK(C18DA-gGlu-OEG1-OEG2-)
PEADATPEELQRYYVSLRHYYNWLTRQRY-NH2 (sequence and
structure below disclosed as SEQ ID NO: 18)

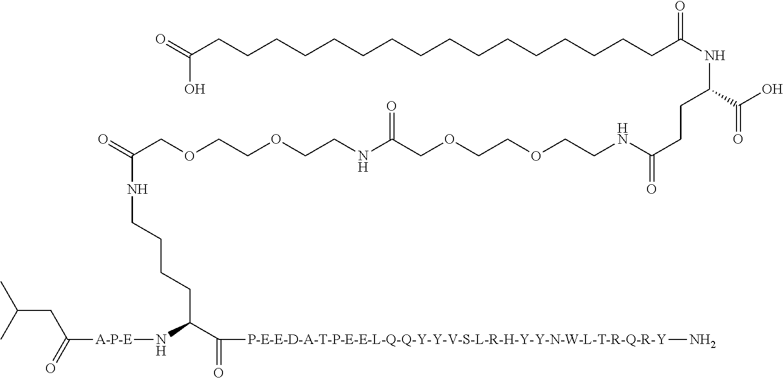

MW (calculated): 4943.6 Da

Synthesis and purification methods: S01; P01

LCMS: A01; Rt: 9.96 min; m/3: 1648.0 m/4: − m/5: −

### Compound 15

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,17I,18Q,21E,22V,28Y,30W,31L]hPYY(4-36)

(SEQ ID NO: 19)
iVal-APEK(C18DA-gGlu-OEG1-OEG2-)
PEEDASPEEIQRYEVSLRHYYNWLTRQRY-NH2

MW (calculated): 4953.5 Da

Synthesis and purification methods: S01; P01

LCMS: A01; Rt: 9.22 min; m/3: 1652.0 m/4: − m/5: −

### Compound 16

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,13A,18Q,19Q,22V,28Y,30W,31L]hPYY(4-36)

(SEQ ID NO: 20)
iVal-APEK(C18DA-gGlu-OEG1-OEG2-)
PEEDASPEETQRYYVTLRHYYNWLTRQRY-NH2

MW (calculated): 4989.6 Da

Synthesis and purification methods: S01; P01

LCMS: A01; Rt: 9.28 min; m/3: − m/4: 1248.0 m/5: −

### Compound 17

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,15A,18Q,22V,28Y,30W,31L]hPYY(4-36)

(SEQ ID NO: 21)
iVal-APEK(C18DA-gGlu-OEG1-OEG2-)
PEEDASPAELQRYYVSLRHYYNWLTRQRY-NH2

MW (calculated): 4929.6 Da

Synthesis and purification methods: S01; P01

LCMS: A01; Rt: 9.77 min; m/3: 1643.0 m/4: − m/5: −

### Compound 18

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,13E,18Q,19Q,22V,28Y,30W,31L]hPYY(4-36)

(SEQ ID NO: 22)
iVal-APEK(C18DA-gGlu-OEG1-OEG2-)
PEEDAEPEELQQYYVSLRHYYNWLTRQRY-NH2

MW (calculated): 5001.6 Da

Synthesis and purification methods: S01; P01

LCMS: A01; Rt: 10.27 min; m/3: − m/4: 1251.0 m/5: −

### Compound 19

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,10A,18Q,19Q,22V,28Y,30W,31L]hPYY(4-36)

(SEQ ID NO: 23)
iVal-APEK(C18DA-gGlu-OEG1-OEG2-)
PEADASPEELQQYYVSLRHYYNWLTRQRY-NH2

MW (calculated): 4901.5 Da

Synthesis and purification methods: S01; P01

LCMS: A01; Rt: 9.90 min; m/3: 1634.0 m/4: − m/5: −

### Compound 20

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,13A,18Q,22V,23E,28Y,30W,31L]hPYY(4-36)

(SEQ ID NO: 24)
iVal-APEK(C18DA-gGlu-OEG1-OEG2-)
PEEDAAPEELQRYYVELRHYYNWLTRQRY-NH2

MW (calculated): 5013.6 Da

Synthesis and purification methods: S01; P01

LCMS: A01; Rt: 10.72 min; m/3: − m/4: 1253.0 m/5: −

### Compound 21

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,18Q,19K,22V,28Y,30W,31L]hPYY(4-36)

(SEQ ID NO: 25)
iVal-APEK(C18DA-gGlu-OEG1-OEG2-)
PEEDASPEELQKYYVSLRHYYNWLTRQRY-NH2

MW (calculated): 4959.6 Da

Synthesis and purification methods: S01; P01

LCMS: A01; Rt: 9.62 min; m/3: − m/4: 1241.0 m/5: −

### Compound 22

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,18Q,22V,23E,24I,28Y,30W,31L]hPYY(4-36)

(SEQ ID NO: 26)
iVal-APEK(C18DA-gGlu-OEG1-OEG2-)
PEEDASPEELQRYYTSLRHYYNWLTRQRY-NH2

MW (calculated): 4989.6 Da

Synthesis and purification methods: S01; P01

LCMS: A01; Rt: 9.63 min; m/3: 1664.0 m/4: − m/5: −

### Compound 23

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,6A,7K,9E,17I,18Q,22V,28Y,30W,31L]hPYY(4-36)

iVal-APAK(C18DA-gGlu-OEG1-OEG2-)
PEEDASPEEIQRYYVSLRHYYNWLTRQRY-NH2
(sequence and structure below disclosed
as SEQ ID NO: 27)

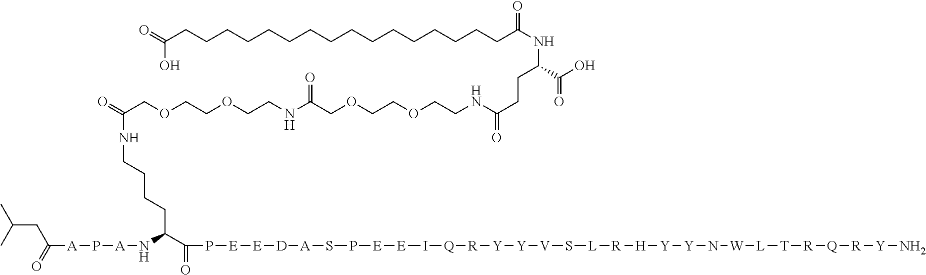

MW (calculated): 4929.6 Da

Synthesis and purification methods: S01; P01

LCMS: A01; Rt: 9.82 min; m/3: − m/4: 1233.0 m/5: −

### Compound 24

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,18Q,22V,26K,28Y,30W,31L]hPYY(4-36)

(SEQ ID NO: 28)
iVal-APEK(C18DA-gGlu-OEG1-OEG2-)
PEEDASPEELQRYYVSLRKYYNWLTRQRY-NH2

MW (calculated): 4978.6 Da

Synthesis and purification methods: S01; P01

LCMS: A01; Rt: 9.70 min; m/3: 1661.0 m/4: − m/5: −

### Compound 25

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,14A,18Q,22V,28Y,30W,31L]hPYY(4-36)

(SEQ ID NO: 29)
iVal-APEK(C18DA-gGlu-OEG1-OEG2-)
PEEEASAEELQRYYVSLRHYYNWLTRQRY-NH2

MW (calculated): 4975.6 Da

Synthesis and purification methods: S01; P01

LCMS: A01; Rt: 10.74 min; m/3: − m/4: 1245.0 m/5: −

### Compound 26

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,14A,18Q,22V,28Y,30W,31L]hPYY(4-36)

iVal-APEK(C18DA-gGlu-OEG1-OEG2-)
PPEDATPEELQRYYVSLRHYYNWLTRQRY-NH2
(sequence and structure below disclosed
as SEQ ID NO: 30)

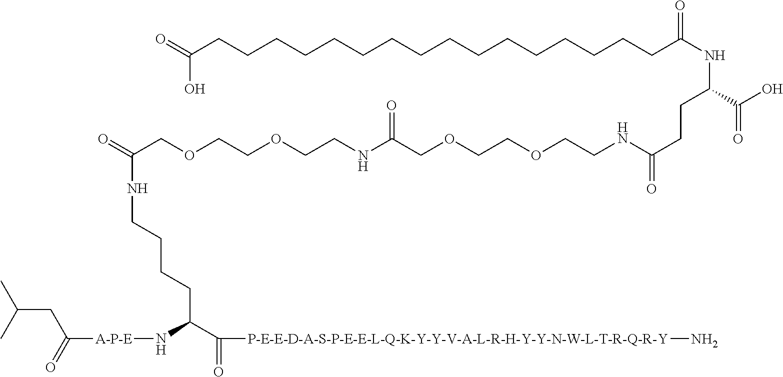

MW (calculated): 4969.6 Da

Synthesis and purification methods: S01; P01

LCMS: A01; Rt: 9.74 min; m/3: − m/4: 1243.0 m/5: −

### Compound 27

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9P,13E,18Q,22V,28Y,30W,31L]hPYY(4-36)

(SEQ ID NO: 31)
iVal-APEK(C18DA-gGlu-OEG1-OEG2-)
PPEDAEPEELQRYYVSLRHYYNWLTRQRY-NH2

MW (calculated): 4997.6 Da

Synthesis and purification methods: S01; P01

LCMS: A01; Rt: 10.11 min; m/3: 1667.0 m/4: − m/5: −

### Compound 28

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,14A,18Q,22V,28Y,30W,31L]hPYY(4-36)

(SEQ ID NO: 32)
iVal-APEK(C18DA-gGlu-OEG1-OEG2-)
PEEDTSPEELQRYYVELRHYYNWLTRQRY-NH2

MW (calculated): 5059.7 Da

Synthesis and purification methods: S01; P01

LCMS: A01; Rt: 9.77 min; m/3: 1688.0 m/4: − m/5: −

### Compound 29

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,10A,11P,18Q,22V,23E,28Y,30W,31L]hPYY(4-36)

iVal-APEK(C18DA-gGlu-OEG1-OEG2-)
PEAPASPEELQRYYVELRHYYNWLTRQRY-NH2
(sequence and structure below disclosed
as SEQ ID NO: 33)

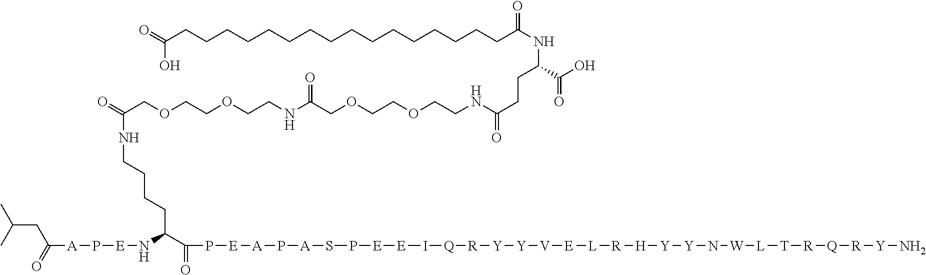

MW (calculated): 4953.6 Da

Synthesis and purification methods: S01; P01

LCMS: A01; Rt: 9.97 min; m/3: − m/4: 1239.0 m/5: −

### Compound 30

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,10A,14E,18Q,22V,28Y,30W,31L]hPYY(4-36)

(SEQ ID NO: 34)
iVal-APEK(C18DA-gGlu-OEG1-OEG2-)
PEADASEEELQRYYVSLRHYYNWLTRQRY-NH2

MW (calculated): 4961.6 Da

Synthesis and purification methods: S01; P01

LCMS: A01; Rt: 10.47 min; m/3: − m/4: 1241.0 m/5: −

### Compound 31

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,18Q,22V,23E,24I,28Y,30W,31L]hPYY(4-36)

(SEQ ID NO: 35)
iVal-APEK(C18DA-gGlu-OEG1-OEG2-)
PEEDASPEETQRYYVALRHYYNWLTRQRY-NH2

MW (calculated): 4959.6 Da

Synthesis and purification methods: S01; P01

LCMS: A01; Rt: 9.26 min; m/3: 1654.0 m/4: − m/5: −

### Compound 32

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,18Q,22V,23E,24I,28Y,30W,31L]hPYY(4-36)

(SEQ ID NO: 36)
iVal-APEK(C18DA-gGlu-OEG1-OEG2-)
PEEDTSPEELQRYEVSLRHYYNWLTRQRY-NH2

MW (calculated): 4983.6 Da

Synthesis and purification methods: S01; P01

LCMS: A01; Rt: 9.46 min; m/3: − m/4: 1247.0 m/5: −

### Compound 33

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11P,18Q,22V,23E,28Y,30W,31L]hPYY(4-36)

(SEQ ID NO: 37)
iVal-APEK(C18DA-gGlu-OEG1-OEG2-)
PEEPASPEELQRYYVELRHYYNWLTRQRY-NH2

MW (calculated): 5011.7 Da

Synthesis and purification methods: S01; P01

LCMS: A01; Rt: 9.85 min; m/3: − m/4: 1254.0 m/5: −

### Compound 34

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,14A,18Q,22V,28Y,30W,31L]hPYY(4-36)

(SEQ ID NO: 38)
iVal-APEK(C18DA-gGlu-OEG1-OEG2-)
PEEDASPEELQRYYVSTRHYYNWLTRQRY-NH2

MW (calculated): 4975.6 Da

Synthesis and purification methods: S01; P01

LCMS: A01; Rt: 9.20 min; m/3: 1660.0 m/4: − m/5: −

### Compound 35

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,14A,18Q,22V,28Y,30W,31L]hPYY(4-36)

(SEQ ID NO: 39)
iVal-APEK(C18DA-gGlu-OEG1-OEG2-)
PEEDAAPEETQRYYVSLRHYYNWLTRQRY-NH2

MW (calculated): 4959.6 Da

Synthesis and purification methods: S01; P01

LCMS: A01; Rt: 9.45 min; m/3: − m/4: 1240.0 m/5: −

### Compound 36

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,8A,9E,13E,18Q,22V,28Y,30W,31L]hPYY(4-36)

(SEQ ID NO: 40)
iVal-APEK(C18DA-gGlu-OEG1-OEG2-)
AEEDAEPEELQRYYVSLRHYYNWLTRQRY-NH2

MW (calculated): 5003.6 Da

Synthesis and purification methods: S01; P01

LCMS: A01; Rt: 10.34 min; m/3: − m/4: 1252.0 m/5: −

### Compound 37

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,14A,18Q,22V,28Y,30W,31L]hPYY(4-36)

iVal-APEK(C18DA-gGlu-OEG1-OEG2-)
PEEDASPEETQRYEVSLRHYYNWLTRQRY-NH2 (sequence
and structure below disclosed as SEQ ID NO: 41)

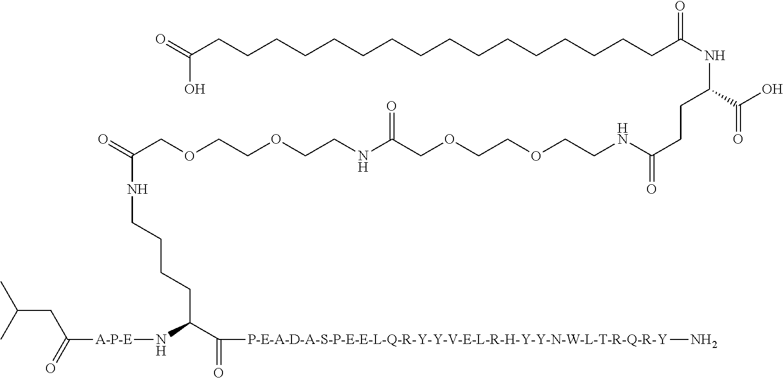

MW (calculated): 4941.5 Da

Synthesis and purification methods: S01; P01

LCMS: A01; Rt: 8.97 min; m/3: 1649.0 m/4: − m/5: −

### Compound 38

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9P,18Q,22V,23A,28Y,30W,31L]hPYY(4-36)

(SEQ ID NO: 42)
iVal-APEK(C18DA-gGlu-OEG1-OEG2-)
PPEDASPEELQRYYVALRHYYNWLTRQRY-NH2

MW (calculated): 4939.6 Da

Synthesis and purification methods: S01; P01

LCMS: A01; Rt: 9.75 min; m/3: − m/4: 1236.0 m/5: −

### Compound 39

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9P,10A,13E,18Q,22V,28Y,30W,31L]hPYY(4-36)

(SEQ ID NO: 43)
iVal-APEK(C18DA-gGlu-OEG1-OEG2-)
PPADAEPEELQRYYVSLRHYYNWLTRQRY-NH2

MW (calculated): 4939.6 Da

Synthesis and purification methods: S01; P01

LCMS: A01; Rt: 10.18 min; m/3: − m/4: 1236.0 m/5: −

### Compound 40

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,14A,18Q,22V,28Y,30W,31L]hPYY(4-36)

(SEQ ID NO: 44)
iVal-APEK(C18DA-gGlu-OEG1-OEG2-)
PEEDATPEETQRYYVSLRHYYNWLTRQRY-NH2

MW (calculated): 4989.6 Da

Synthesis and purification methods: S01; P01

LCMS: A01; Rt: 8.84 min; m/3: 1664.0 m/4: − m/5: −

### Compound 41

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,14A,18Q,22V,28Y,30W,31L]hPYY(4-36)

(SEQ ID NO: 45)
iVal-APEK(C18DA-gGlu-OEG1-OEG2-)
PEEEATPEELQRYYVSLRHYYNWLTRQRY-NH2

MW (calculated): 5015.7 Da

Synthesis and purification methods: S01; P01

LCMS: A01; Rt: 9.93 min; m/3: − m/4: 1255.0 m/5: −

### Compound 42

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,14A,18Q,22V,28Y,30W,31L]hPYY(4-36)

(SEQ ID NO: 46)
iVal-APEK(C18DA-gGlu-OEG1-OEG2-)
PEEAASPEELQRYYVSTRHYYNWLTRQRY-NH2

MW (calculated): 4931.5 Da

Synthesis and purification methods: S01; P01

LCMS: A01; Rt: 9.35 min; m/3: − m/4: 1234.0 m/5: −

### Compound 43

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,10A,11P,18Q,22V,28Y,30W,31L]hPYY(4-36)

(SEQ ID NO: 47)
iVal-APEK(C18DA-gGlu-OEG1-OEG2-)
PEAPASPEELQRYYVSLRHYYNWLTRQRY-NH2

MW (calculated): 4911.6 Da

Synthesis and purification methods: S01; P01

LCMS: A01; Rt: 9.86 min; m/3: 1638.0 m/4: − m/5: −

### Compound 44

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,18Q,22V,28Y,30W,31I]hPYY(4-36)

(SEQ ID NO: 48)
iVal-APEK(C18DA-gGlu-OEG1-OEG2-)
PEEEASPEELQRYYVSLRHYYNWITRQRY-NH2

MW (calculated): 5001.6 Da

Synthesis and purification methods: S01; P01

LCMS: A01; Rt: 9.66 min; m/3: 1668.0 m/4: − m/5: −

### Compound 45

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,16S,18Q,22V,28Y,30W,31L]hPYY(4-36)

(SEQ ID NO: 49)
iVal-APEK(C18DA-gGlu-OEG1-OEG2-)
PEEEASPESLQRYYVSLRHYYNWLTRQRY-NH2

MW (calculated): 4959.6 Da

Synthesis and purification methods: S01; P01

LCMS: A01; Rt: 9.96 min; m/3: − m/4: 1240.0 m/5: −

### Compound 46

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,18Q,22V,28Y,30W,31L]hPYY(4-36)

(SEQ ID NO: 50)
iVal-APEK(C18DA-gGlu-OEG1-OEG2-)
PEEEASPEELQRYYVSLRHYYNWLTRQRY-NH2

MW (calculated): 5001.6 Da

Synthesis and purification methods: S01; P01

LCMS: A01; Rt: 9.80 min; m/3: 1668.0 m/4: − m/5: −

### Compound 47

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9P,10A,18Q,22V,28Y,30W,31L]hPYY(4-36)

(SEQ ID NO: 51)
iVal-APEK(C18DA-gGlu-OEG1-OEG2-)
PPADASPEELQRYYVSLRHYYNWLTRQRY-NH2

MW (calculated): 4897.6 Da

Synthesis and purification methods: S01; P01

LCMS: A01; Rt: 9.60 min; m/3: − m/4: 1224.0 m/5: −

### Compound 48

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,14A,18Q,22V,28Y,30W,31L]hPYY(4-36)

(SEQ ID NO: 52)
iVal-APEK(C18DA-gGlu-OEG1-OEG2-)
PEEDTEPEELQRYYVSLRHYYNWLTRQRY-NH2

MW (calculated): 5059.7 Da

Synthesis and purification methods: S01; P01

LCMS: A01; Rt: 10.08 min; m/3: 1688.0 m/4: − m/5: −

### Compound 49

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,18Q,19Q,22V,23E,28Y,30W,31L]hPYY(4-36)

(SEQ ID NO: 53)
iVal-APEK(C18DA-gGlu-OEG1-OEG2-)
PEEDASPEELQQYYVELRHYYNWLTRQRY-NH2

MW (calculated): 5001.6 Da

Synthesis and purification methods: S01; P01

LCMS: A01; Rt: 9.95 min; m/3: − m/4: 1251.0 m/5: −

### Compound 50

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,14A,18Q,22V,28Y,30W,31I]hPYY(4-36)

(SEQ ID NO: 54)
iVal-APEK(C18DA-gGlu-OEG1-OEG2-)
PEEDASAEELQRYYVSLRHYYNWITRQRY-NH2

MW (calculated): 4961.6 Da

Synthesis and purification methods: S01; P01

LCMS: A01; Rt: 10.33 min; m/3: − m/4: 1241.0 m/5: −

### Compound 51

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,8A,9E,11E,18Q,22V,28Y,30W,31L]hPYY(4-36)

(SEQ ID NO: 55)
iVal-APEK(C18DA-gGlu-OEG1-OEG2-)
AEEEASPEELQRYYVSLRHYYNWLTRQRY-NH2

MW (calculated): 4975.6 Da

Synthesis and purification methods: S01; P01

LCMS: A01; Rt: 10.07 min; m/3: 1659.0 m/4: − m/5: −

### Compound 52

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,8A,9E,14E,18Q,22V,28Y,30W,31L]hPYY(4-36)

(SEQ ID NO: 56)
iVal-APEK(C18DA-gGlu-OEG1-OEG2-)
AEEDASEEELQRYYVSLRHYYNWLTRQRY-NH2

MW (calculated): 4993.6 Da

Synthesis and purification methods: S01; P01

LCMS: A01; Rt: 10.76 min; m/3: − m/4: 1249.0 m/5: −

### Compound 53

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,10A,17I,18Q,22V,28Y,30W,31L]hPYY(4-36)

iVal-APEK(C18DA-gGlu-OEG1-OEG2-)
PEADASPEEIQRYYVSLRHYYNWLTRQRY-NH2 (sequence
and structure below disclosed as SEQ ID NO: 57)

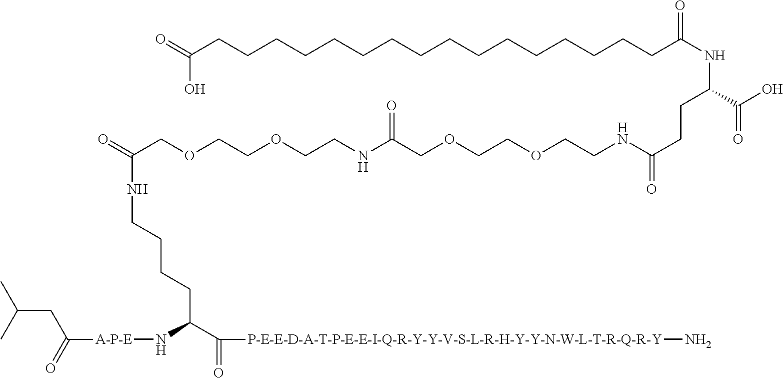

MW (calculated): 4929.6 Da

Synthesis and purification methods: S01; P01

LCMS: A01; Rt: 9.72 min; m/3: − m/4: 1233.0 m/5: −

### Compound 54

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,18Q,22V,28Y,30W,31L]hPYY(4-36)

(SEQ ID NO: 58)
iVal-APEK(C18DA-gGlu-OEG1-OEG2-)
PEEDASPEELQRYYVSLRHYYNWLTRQRY-NH2

MW (calculated): 4987.6 Da

Synthesis and purification methods: S01; P01

LCMS: A01; Rt: 9.77 min; m/3: − m/4: − m/5: 997.0

### Compound 55

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,17A,18Q,22V,28Y,30W,31L]hPYY(4-36)

(SEQ ID NO: 59)
iVal-APEK(C18DA-gGlu-OEG1-OEG2-)
PEEDASPEEAQRYYVSLRHYYNWLTRQRY-NH2

MW (calculated): 4945.5 Da

Synthesis and purification methods: S01; P01

LCMS: A01; Rt: 9.25 min; m/3: 1649.0 m/4: − m/5: −

### Compound 56

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,10A,13E,18Q,22V,28Y,30W,31L]hPYY(4-36)

(SEQ ID NO: 60)
iVal-APEK(C18DA-gGlu-OEG1-OEG2-)
PEADAEPEELQRYYVSLRHYYNWLTRQRY-NH2

MW (calculated): 4971.6 Da

Synthesis and purification methods: S01; P01

LCMS: A01; Rt: 10.17 min; m/3: − m/4: 1244.0 m/5: −

### Compound 57

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,14A,17I,18Q,22V,28Y,30W,31L]hPYY(4-36)

(SEQ ID NO: 61)
iVal-APEK(C18DA-gGlu-OEG1-OEG2-)
PEEDASAEEIQRYYVSLRHYYNWLTRQRY-NH2

MW (calculated): 4961.6 Da

Synthesis and purification methods: S01; P01

LCMS: A01; Rt: 10.50 min; m/3: − m/4: 1241.0 m/5: −

### Compound 58

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,18Q,19Q,22V,28Y,30W,31L]hPYY(4-36)

(SEQ ID NO: 62)
iVal-APEK(C18DA-gGlu-OEG1-OEG2-)
PEEDASPEELQQYYVSLRHYYNWLTRQRY-NH2

MW (calculated): 4959.6 Da

Synthesis and purification methods: S01; P01

LCMS: A01; Rt: 9.87 min; m/3: − m/4: 1241.0 m/5: −

### Compound 59

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,14A,18Q,22V,28Y,30W,31L]hPYY(4-36)

(SEQ ID NO: 63)
iVal-APEK(C18DA-gGlu-OEG1-OEG2-)
PEEDASPEETEQYYVSLRHYYNWLTRQRY-NH2

MW (calculated): 4948.5 Da

Synthesis and purification methods: S02; P02

LCMS: A02; Rt: 13.67 min; m/3: 1651.0 m/4: 1238.0 m/5: 990.5

### Compound 60

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11A,18E,19Q,22V,28Y,30W,31L]hPYY(4-36)

(SEQ ID NO: 64)
iVal-APEK(C18DA-gGlu-OEG1-OEG2-)
PEEAASPEELEQYYVSLRHYYNWLTRQRY-NH2

MW (calculated): 4916.5 Da

Synthesis and purification methods: S01; P01

LCMS: A01; Rt: 10.30 min; m/3: − m/4: 1230.0 m/5: −

### Compound 61

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9P,17I,18Q,22V,28Y,30W,31L]hPYY(4-36)

(SEQ ID NO: 65)
iVal-APEK(C18DA-gGlu-OEG1-OEG2-)
PPEDASPEEIQRYYVSLRHYYNWLTRQRY-NH2

MW (calculated): 4955.6 Da

Synthesis and purification methods: S01; P01

LCMS: A01; Rt: 9.51 min; m/3: − m/4: 1240.0 m/5: −

### Compound 62

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,18E,22V,28Y,30W,31L]hPYY(4-36)

(SEQ ID NO: 66)
iVal-APEK(C18DA-gGlu-OEG1-OEG2-)
PGEDASPEELERYYVSLRHYYNWLTRQRY-NH2

MW (calculated): 4916.5 Da

Synthesis and purification methods: S01; P01

LCMS: A01; Rt: 9.91 min; m/3: − m/4: − m/5: 983.0

### Compound 63

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,18Q,22V,26A,28Y,30W,31L]hPYY(4-36)

(SEQ ID NO: 67)
iVal-APEK(C18DA-gGlu-OEG1-OEG2-)
PEEDASPEELQRYYVSLRAYYNWLTRQRY-NH2

MW (calculated): 4921.5 Da

Synthesis and purification methods: S01; P01

LCMS: A01; Rt: 10.10 min; m/3: 1641.0 m/4: − m/5: −

### Compound 64

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,18Q,22V,28Y,30W,31L]hPYY(4-36)

(SEQ ID NO: 68)
iVal-APEK(C18DA-gGlu-OEG1-OEG2-)
PGEDASPEELQRYYVSLRHYYNWLTRQRY-NH2

MW (calculated): 4915.5 Da

Synthesis and purification methods: S01; P01

LCMS: A01; Rt: 9.65 min; m/3: 1639.0 m/4: − m/5: −

### Compound 65

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,18Q,22V,23E,24I,28Y,30W,31L]hPYY(4-36)

iVal-APEK(C18DA-gGlu-OEG1-OEG2-)
PEEDASPEELQRYYVEIRHYYNWLTRQRY-NH2 (sequence
and structure below disclosed as SEQ ID NO: 69)

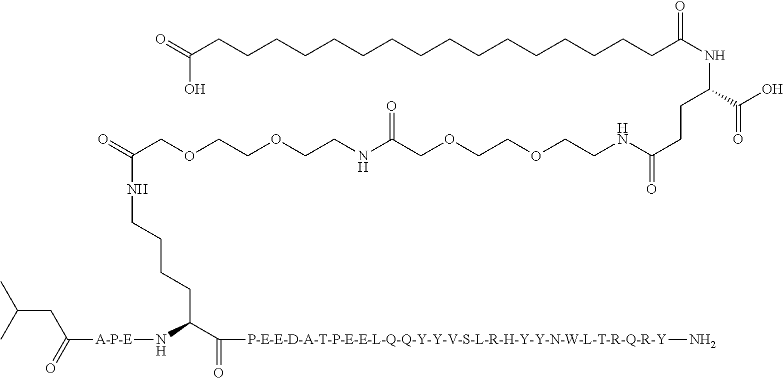

MW (calculated): 5029.6 Da

Synthesis and purification methods: S01; P01

LCMS: A01; Rt: 9.82 min; m/3: − m/4: 1258.0 m/5: −

### Compound 66

### N{alpha-4}-(3-methylbutanoyl)-N {epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,18Q,22V,23E,24I,28Y,30W,31L]hPYY(4-36)

(SEQ ID NO: 70)
iVal-APEK(C18DA-gGlu-OEG1-OEG2-)
PEEDASAEETQRYYVSLRHYYNWLTRQRY-NH2

MW (calculated): 4949.5 Da

Synthesis and purification methods: S01; P01

LCMS: A01; Rt: 9.61 min; m/3: − m/4: 1238.0 m/5: −

### Compound 67

### N{alpha-4}-(3-methylbutanoyl)-N {epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,12S,13A,18Q,22V,28Y,30W,31L]hPYY(4-36)

(SEQ ID NO: 71)
iVal-APEK(C18DA-gGlu-OEG1-OEG2-)
PEEDSAPEELQRYYVSLRHYYNWLTRQRY-NH2

MW (calculated): 4987.6 Da

Synthesis and purification methods: S01; P01

LCMS: A01; Rt: 10.60 min; m/3: − m/4: 1248.0 m/5: −

### Compound 68

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,18Q,19A,22V,28Y,30W,31L]hPYY(4-36)

(SEQ ID NO: 72)
iVal-APEK(C18DA-gGlu-OEG1-OEG2-)
PEEEASPEELQAYYVSLRHYYNWLTRQRY-NH2

MW (calculated): 4916.5 Da

Synthesis and purification methods: S01; P01

LCMS: A01; Rt: 10.13 min; m/3: − m/4: 1230.0 m/5: −

### Compound 69

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,12S,14A,18Q,22V,28Y,30W,31L]hPYY(4-36)

(SEQ ID NO: 73)
iVal-APEK(C18DA-gGlu-OEG1-OEG2-)
PEEDSSAEELQRYYVSLRHYYNWLTRQRY-NH2

MW (calculated): 4977.6 Da

Synthesis and purification methods: S01; P01

LCMS: A01; Rt: 10.26 min; m/3: − m/4: 1245.0 m/5: −

### Compound 70

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11A,18Q,21E,22V,28Y,30W,31L]hPYY(4-36)

(SEQ ID NO: 74)
iVal-APEK(C18DA-gGlu-OEG1-OEG2-)
PEEAASPEELQRYEVSLRHYYNWLTRQRY-NH2

MW (calculated): 4909.5 Da

Synthesis and purification methods: S01; P01

LCMS: A01; Rt: 9.55 min; m/3: 1638.0 m/4: − m/5: −

### Compound 71

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,15S,18Q,22V,28Y,30W,31L]hPYY(4-36)

(SEQ ID NO: 75)
iVal-APEK(C18DA-gGlu-OEG1-OEG2-)
PEEEASPSELQRYYVSLRHYYNWLTRQRY-NH2

MW (calculated): 4959.6 Da

Synthesis and purification methods: S01; P01

LCMS: A01; Rt: 9.96 min; m/3: − m/4: 1242.0 m/5: −

### Compound 72

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,12G,18Q,22V,28Y,30W,31L]hPYY(4-36)

(SEQ ID NO: 76)
iVal-APEK(C18DA-gGlu-OEG1-OEG2-)
PEEEGSPEELQRYYVSLRHYYNWLTRQRY-NH2

MW (calculated): 4987.6 Da

Synthesis and purification methods: S01; P01

LCMS: A01; Rt: 9.87 min; m/3: − m/4: 1248.0 m/5: −

### Compound 73

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,18Q,22V,26A,28Y,30W,31L]hPYY(4-36)

(SEQ ID NO: 77)
iVal-APEK(C18DA-gGlu-OEG1-OEG2-)
PEEEASPEELQRYYVSLRAYYNWLTRQRY-NH2

MW (calculated): 4935.6 Da

Synthesis and purification methods: S03; P02

LCMS: A02; Rt: 12.23 min; m/3: 1646.8 m/4: 1235.0 m/5: 988.0

### Compound 74

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,14A,18Q,22V,23A,28Y,30W,31L]hPYY(4-36)

PEEDASAEELQRYYVALRHYYNWLTRQRY-NH2 (sequence
and structure below disclosed as SEQ ID NO: 78)

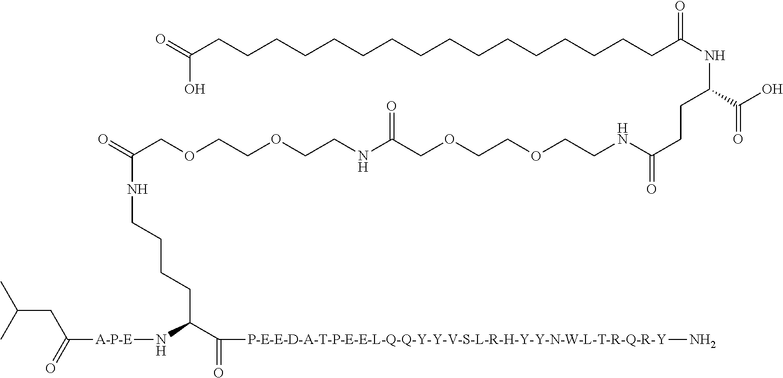

MW (calculated): 4945.6 Da

Synthesis and purification methods: S01; P01

LCMS: A01; Rt: 10.72 min; m/3: 1650.0 m/4: − m/5: −

### Compound 75

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,14A,18Q,22V,28Y,30W,31L]hPYY(4-36)

(SEQ ID NO: 79)
iVal-APEK(C18DA-gGlu-OEG1-OEG2-)
PEEDASAEELQRYYVTLRHYYNWLTRQRY-NH2

MW (calculated): 4975.6 Da

Synthesis and purification methods: S01; P01

LCMS: A01; Rt: 10.74 min; m/3: − m/4: 1244.0 m/5: −

### Compound 76

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,18Q,22V,28Y,30A,31L]hPYY(4-36)

iVal-APEK(C18DA-gGlu-OEG1-OEG2-)
PGEDASPEELQRYYVSLRHYYNALTRQRY-NH2 (sequence
and structure below disclosed as SEQ ID NO: 80)

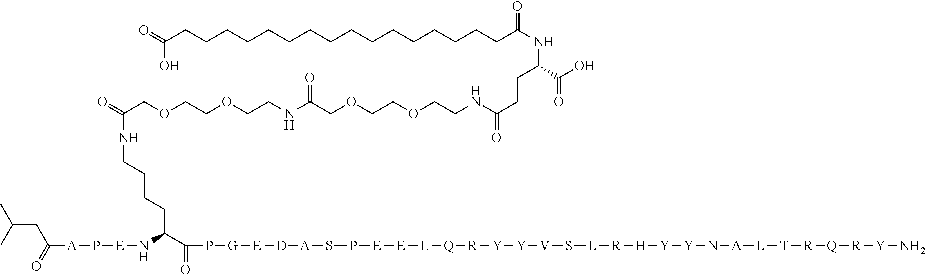

MW (calculated): 4800.4 Da

Synthesis and purification methods: S01; P01

LCMS: A01; Rt: 9.12 min; m/3: − m/4: 1201.0 m/5: −

### Compound 77

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,14E,18Q,22V,23E,28Y,30W,31L]hPYY(4-36)

(SEQ ID NO: 81)
iVal-APEK(C18DA-gGlu-OEG1-OEG2-)
PEEDASEEELQRYYVELRHYYNWLTRQRY-NH2

MW (calculated): 5061.6 Da

Synthesis and purification methods: S01; P01

LCMS: A01; Rt: 10.44 min; m/3: − m/4: 1266.0 m/5: −

### Compound 78

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,18Q,22V,23E,24I,28Y,30W,31L]hPYY(4-36)

(SEQ ID NO: 82)
iVal-APEK(C18DA-gGlu-OEG1-OEG2-)
PEEDAEPEELQQYYVTLRHYYNWLTRQRY-NH2

MW (calculated): 5043.7 Da

Synthesis and purification methods: S03; P02

LCMS: A02; Rt: 12.75 min; m/3: 1682.6 m/4: 1262.1 m/5: 1009.7

### Compound 79

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,14E,18Q,22V,28Y,30W,31L]hPYY(4-36)

(SEQ ID NO: 83)
iVal-APEK(C18DA-gGlu-OEG1-OEG2-)
PEEEASEEELQRYYVSLRHYYNWLTRQRY-NH2

MW (calculated): 5033.6 Da

Synthesis and purification methods: S01; P01

LCMS: A01; Rt: 10.66 min; m/3: − m/4: 1260.0 m/5: −

### Compound 80

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,14A,18Q,22V,28Y,30W,31L]hPYY(4-36)

(SEQ ID NO: 84)
iVal-APEK(C18DA-gGlu-OEG1-OEG2-)
PEEEASPEETQRYYVSLRHYYNWLTRQRY-NH2

MW (calculated): 4989.6 Da

Synthesis and purification methods: S01; P01

LCMS: A01; Rt: 9.19 min; m/3: − m/4: 1248.0 m/5: −

### Compound 81

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,18Q,22V,24I,28Y,30W,31L]hPYY(4-36)

(SEQ ID NO: 85)
iVal-APEK(C18DA-gGlu-OEG1-OEG2-)
PEEDASPEELQRYYVSIRHYYNWLTRQRY-NH2

MW (calculated): 4987.6 Da

Synthesis and purification methods: S01; P01

LCMS: A01; Rt: 9.72 min; m/3: 1664.0 m/4: − m/5: −

### Compound 82

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,16A,18Q,22V,23A,28Y,30W,31L]hPYY(4-36)

(SEQ ID NO: 86)
iVal-APEK(C18DA-gGlu-OEG1-OEG2-)
PEEDASPEALQRYYVALRHYYNWLTRQRY-NH2

MW (calculated): 4913.6 Da

Synthesis and purification methods: S01; P01

LCMS: A01; Rt: 10.28 min; m/3: − m/4: 1229.0 m/5: −

### Compound 83

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,10A,15A,18Q,22V,28Y,30W,31L]hPYY(4-36)

iVal-APEK(C18DA-gGlu-OEG1-OEG2-)
PEADASPAELQRYYVSLRHYYNWLTRQRY-NH2 (sequence
and structure below disclosed as SEQ ID NO: 87)

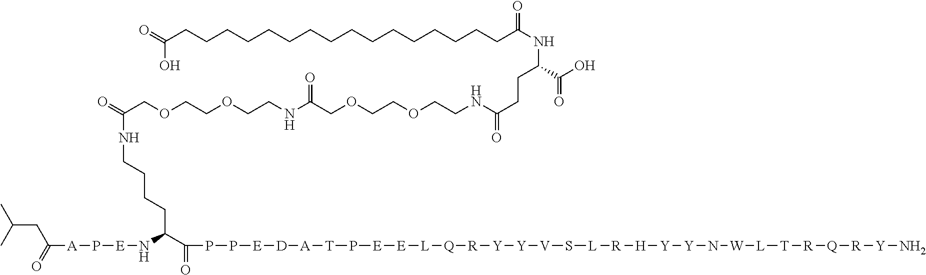

MW (calculated): 4871.5 Da

Synthesis and purification methods: S01; P01

LCMS: A01; Rt: 9.85 min; m/3: − m/4: 1219.0 m/5: −

### Compound 84

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,17I,18Q,22V,28Y,30W,31L]hPYY(4-36)

iVal-APEK(C18DA-gGlu-OEG1-OEG2-)
PEEDASPEEIQRYYVSLRHYYNWLTRQRY-NH2 (sequence
and structure below disclosed as SEQ ID NO: 88)

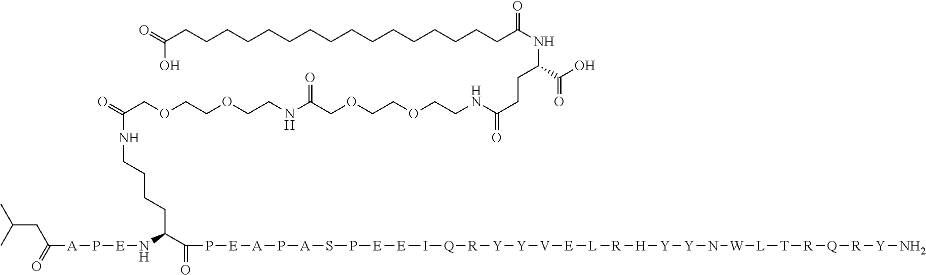

MW (calculated): 4987.6 Da

Synthesis and purification methods: S01; P01

LCMS: A01; Rt: 9.60 min; m/3: 1664.0 m/4: − m/5: −

### Compound 85

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,18Q,21A,22V,28Y,30W,31L]hPYY(4-36)

(SEQ ID NO: 89)
iVal-APEK(C18DA-gGlu-OEG1-OEG2-)
PEEDASPEELQRYAVSLRHYYNWLTRQRY-NH2

MW (calculated): 4895.5 Da

Synthesis and purification methods: S01; P01

LCMS: A01; Rt: 9.82 min; m/3: 1633.0 m/4: − m/5: −

### Compound 86

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,10A,18Q,21E,22V,28Y,30W,31L]hPYY(4-36)

(SEQ ID NO: 90)
iVal-APEK(C18DA-gGlu-OEG1-OEG2-)
PEADASPEELQRYEVSLRHYYNWLTRQRY-NH2

MW (calculated): 4895.5 Da

Synthesis and purification methods: S01; P01

LCMS: A01; Rt: 9.44 min; m/3: − m/4: 1224.0 m/5: −

### Compound 87

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,12S,15A,18Q,22V,28Y,30W,31L]hPYY(4-36)

iVal-APEK(C18DA-gGlu-OEG1-OEG2-)
PEEDSSPAELQRYYVSLRHYYNWLTRQRY-NH2 (sequence
and structure below disclosed as SEQ ID NO: 91)

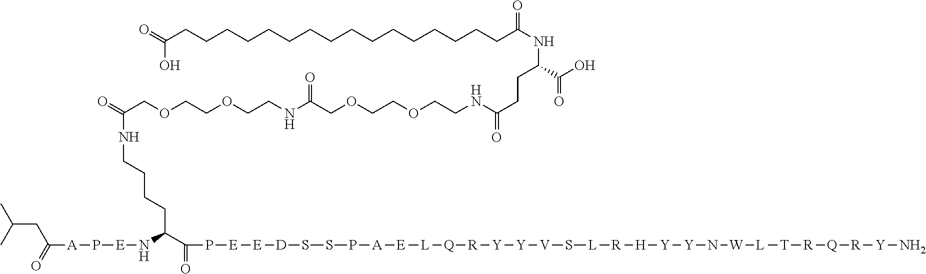

MW (calculated): 4945.6 Da

Synthesis and purification methods: S01; P01

LCMS: A01; Rt: 9.82 min; m/3: − m/4: 1237.0 m/5: −

### Compound 88

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9P,18Q,22V,23E,28Y,30W,31L]hPYY(4-36)

(SEQ ID NO: 92)
iVal-APEK(C18DA-gGlu-OEG1-OEG2-)
PPEDASPEELQRYYVELRHYYNWLTRQRY-NH2

MW (calculated): 4997.6 Da

Synthesis and purification methods: S01; P01

LCMS: A01; Rt: 9.62 min; m/3: − m/4: 1250.0 m/5: −

### Compound 89

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,13E,18Q,22V,28Y,30W,31L]hPYY(4-36)

(SEQ ID NO: 93)
iVal-APEK(C18DA-gGlu-OEG1-OEG2-)
PEEEAEPEELQRYYVSLRHYYNWLTRQRY-NH2

MW (calculated): 5043.7 Da

Synthesis and purification methods: S01; P01

LCMS: A01; Rt: 10.08 min; m/3: − m/4: 1261.0 m/5: −

### Compound 90

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,18Q,21E,22V,28Y,30W,31L]hPYY(4-36)

(SEQ ID NO: 94)
iVal-APEK(C18DA-gGlu-OEG1-OEG2-)
PEEDASPEELQRYEVSLRHYYNWLTRQRY-NH2

MW (calculated): 4953.5 Da

Synthesis and purification methods: S01; P01

LCMS: A01; Rt: 9.46 min; m/3: 1651.0 m/4: − m/5: −

### Compound 91

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,13P,14A,18Q,22V,23E,28Y,30W,31L]hPYY(4-36)

(SEQ ID NO: 95)
iVal-APEK(C18DA-gGlu-OEG1-OEG2-)
PEEDAPAEELQRYYVELRHYYNWLTRQRY-NH2

MW (calculated): 5013.6 Da

Synthesis and purification methods: S01; P01

LCMS: A01; Rt: 11.44 min; m/3: − m/4: 1254.0 m/5: −

### Compound 92

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,18Q,22V,23E,24I,28Y,30W,31L]hPYY(4-36)

(SEQ ID NO: 96)
iVal-APEK(C18DA-gGlu-OEG1-OEG2-)
PEEDTSPEELQRYYVSLRHYYNWLTRQRY-NH2

MW (calculated): 5017.6 Da

Synthesis and purification methods: S01; P01

LCMS: A01; Rt: 9.76 min; m/3: − m/4: 1256.0 m/5: −

### Compound 93

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,14A,18Q,19K,22V,28Y,30W,31L]hPYY(4-36)

(SEQ ID NO: 97)
iVal-APEK(C18DA-gGlu-OEG1-OEG2-)
PEEDASAEELQKYYVSLRHYYNWLTRQRY-NH2

MW (calculated): 4933.6 Da

Synthesis and purification methods: S03; P02

LCMS: A02; Rt: 12.03 min; m/3: 1646.0 m/4: 1234.3 m/5: 987.4

### Compound 94

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,14A,18Q,22V,28Y,30W,31L]hPYY(4-36)

(SEQ ID NO: 98)
iVal-APEK(C18DA-gGlu-OEG1-OEG2-)
PEEDASPEETQRYYVELRHYYNWLTRQRY-NH2

MW (calculated): 5017.6 Da

Synthesis and purification methods: S01; P01

LCMS: A01; Rt: 9.15 min; m/3: − m/4: 1256.0 m/5: −

### Compound 95

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,15A,17I,18Q,22V,28Y,30W,31L]hPYY(4-36)

(SEQ ID NO: 99)
iVal-APEK(C18DA-gGlu-OEG1-OEG2-)
PEEDASPAEIQRYYVSLRHYYNWLTRQRY-NH2

MW (calculated): 4929.6 Da

Synthesis and purification methods: S01; P01

LCMS: A01; Rt: 9.69 min; m/3: 1644.0 m/4: − m/5: −

### Compound 96

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,10A,18Q,22V,28Y,30W,31L]hPYY(4-36)

(SEQ ID NO: 100)
iVal-APEK(C18DA-gGlu-OEG1-OEG2-)
PEADASPEELQRYYVSLRHYYNWLTRQRY-NH2

MW (calculated): 4929.6 Da

Synthesis and purification methods: S01; P01

LCMS: A01; Rt: 9.70 min; m/3: 1644.0 m/4: − m/5: −

### Compound 97

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,18Q,22V,23A,28Y,30W,31L]hPYY(4-36)

(SEQ ID NO: 101)
iVal-APEK(C18DA-gGlu-OEG1-OEG2-)
PEEDASPEELQRYYVALRHYYNWLTRQRY-NH2

MW (calculated): 4971.6 Da

Synthesis and purification methods: S01; P01

LCMS: A01; Rt: 9.86 min; m/3: − m/4: 1244.0 m/5: −

### Compound 98

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,18Q,22S,23A,28Y,30W,31L]hPYY(4-36)

(SEQ ID NO: 102)
iVal-APEK(C18DA-gGlu-OEG1-OEG2-)
PEEDASPEELQRYYSALRHYYNWLTRQRY-NH2

MW (calculated): 4959.6 Da

Synthesis and purification methods: S01; P01

LCMS: A01; Rt: 9.71 min; m/3: 1654.0 m/4: − m/5: −

### Compound 99

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,13E,14A,18Q,22V,28Y,30W,31L]hPYY(4-36)

(SEQ ID NO: 103)
iVal-APEK(C18DA-gGlu-OEG1-OEG2-)
PEEDAEAEELQRYYVSLRHYYNWLTRQRY-NH2

MW (calculated): 5003.6 Da

Synthesis and purification methods: S01; P01

LCMS: A01; Rt: 10.61 min; m/3: 1668.0 m/4: − m/5: −

### Compound 100

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,6A,7K,9P,18Q,22V,28Y,30W,31L]hPYY(4-36)

(SEQ ID NO: 104)
iVal-APAK(C18DA-gGlu-OEG1-OEG2-)
PPEDASPEELQRYYVSLRHYYNWLTRQRY-NH2

MW (calculated): 4897.6 Da

Synthesis and purification methods: S01; P01

LCMS: A01; Rt: 9.76 min; m/3: − m/4: 1225.0 m/5: −

### Compound 101

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,14A,18Q,22V,28Y,30W,31L]hPYY(4-36)

(SEQ ID NO: 105)
iVal-APEK(C18DA-gGlu-OEG1-OEG2-)
PEEDATPEELQRYYVELRHYYNWLTRQRY-NH2

MW (calculated): 5043.7 Da

Synthesis and purification methods: S01; P01

LCMS: A01; Rt: 9.92 min; m/3: − m/4: 1261.0 m/5: −

### Compound 102

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,14A,18Q,22V,28Y,30W,31L]hPYY(4-36)

(SEQ ID NO: 106)
iVal-APEK(C18DA-gGlu-OEG1-OEG2-)
PEEDASPEELQRYYVTLRHYYNWLTRQRY-NH2

MW (calculated): 5001.6 Da

Synthesis and purification methods: S01; P01

LCMS: A01; Rt: 9.88 min; m/3: 1668.0 m/4: − m/5: −

### Compound 103

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,6A,7K,9E,14E,18Q,22V,28Y,30W,31L]hPYY(4-36)

(SEQ ID NO: 107)
iVal-APAK(C18DA-gGlu-OEG1-OEG2-)
PEEDASEEELQRYYVSLRHYYNWLTRQRY-NH2

MW (calculated): 4961.6 Da

Synthesis and purification methods: S01; P01

LCMS: A01; Rt: 10.03 min; m/3: 1654.0 m/4: − m/5: −

### Compound 104

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,14A,18Q,22V,28Y,30W,31L]hPYY(4-36)

(SEQ ID NO: 108)
iVal-APEK(C18DA-gGlu-OEG1-OEG2-)
PEEDASAEELQRYYVSLRHYYNWLTRQRY-NH2

MW (calculated): 4963.6 Da

Synthesis and purification methods: S01; P01

LCMS: A01; Rt: 10.39 min; m/3: − m/4: 1241.0 m/5: −

### Compound 105

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,13E,18Q,19E,22V,28Y,30W,31L]hPYY(4-36)

(SEQ ID NO: 109)
iVal-APEK(C18DA-gGlu-OEG1-OEG2-)
PEEDAEPEELQEYYVSLRHYYNWLTRQRY-NH2

MW (calculated): 5002.6 Da

Synthesis and purification methods: S01; P01

LCMS: A01; Rt: 10.41 min; m/3: − m/4: 1251.0 m/5: −

### Compound 106

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,18Q,22V,23E,24I,28Y,30W,31L]hPYY(4-36)

iVal-APAK(C18DA-gGlu-OEG1-OEG2-)
PEEDATPEELQRYYVSLRHYYNWLTRQRY-NH2
(sequence and structure below disclosed
as SEQ ID NO: 110)

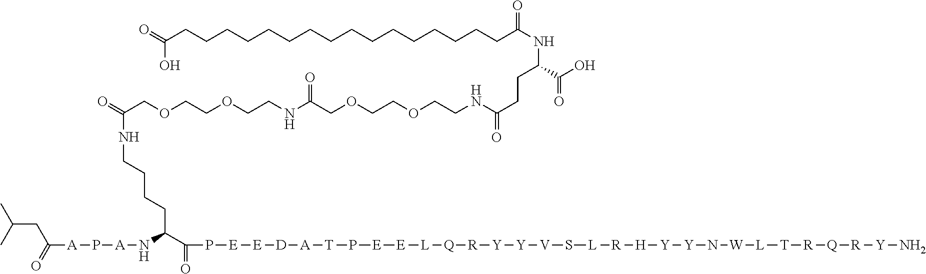

MW (calculated): 4943.6 Da

Synthesis and purification methods: S01; P01

LCMS: A01; Rt: 10.03 min; m/3: − m/4: 1237.0 m/5: −

### Compound 107

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,18Q,19K,22V,23A,28Y,30W,31L]hPYY(4-36)

iVal-APEK(C18DA-gGlu-OEG1-OEG2-)
PEEDASPEELQKYYVALRHYYNWLTRQRY-NH2
(sequence and structure below disclosed
as SEQ ID NO: 111)

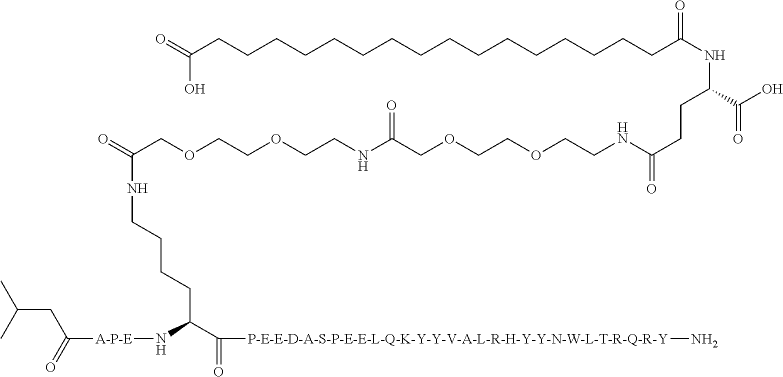

MW (calculated): 4943.6 Da

Synthesis and purification methods: S03; P02

LCMS: A02; Rt: 11.80 min; m/3: 1648.6 m/4: 1236.9 m/5: 989.5

### Compound 108

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,18Q,22V,23E,28Y,30W,31L]hPYY(4-36)

(SEQ ID NO: 112)
iVal-APEK(C18DA-gGlu-OEG1-OEG2-)
PEEDASPEELQRYYVELRHYYNWLTRQRY-NH2

MW (calculated): 5029.6 Da

Synthesis and purification methods: S03; P02

LCMS: A02; Rt: 11.47 min; m/3: 1678.1 m/4: 1258.1 m/5: 1006.7

### Compound 109

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11A,18Q,22V,28Y,30W,31L]hPYY(4-36)

(SEQ ID NO: 113)
iVal-APEK(C18DA-gGlu-OEG1-OEG2-)
PEEAASPEELQRYYVSLRHYYNWLTRQRY-NH2

MW (calculated): 4943.6 Da

Synthesis and purification methods: S01; P01

LCMS: A01; Rt: 9.90 min; m/3: 1649.0 m/4: − m/5: −

### Compound 110

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,13E,17I,18Q,22V,28Y,30W,31L]hPYY(4-36)

(SEQ ID NO: 114)
iVal-APEK(C18DA-gGlu-OEG1-OEG2-)
PEEDAEPEEIQRYYVSLRHYYNWLTRQRY-NH2

MW (calculated): 5029.6 Da

Synthesis and purification methods: S01; P01

LCMS: A01; Rt: 10.10 min; m/3: − m/4: 1258.0 m/5: −

### Compound 111

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,13E,18Q,22V,23E,28Y,30W,31L]hPYY(4-36)

(SEQ ID NO: 115)
iVal-APEK(C18DA-gGlu-OEG1-OEG2-)
PEEDAEPEELQRYYVELRHYYNWLTRQRY-NH2

MW (calculated): 5071.7 Da

Synthesis and purification methods: S01; P01

LCMS: A01; Rt: 10.17 min; m/3: − m/4: 1268.0 m/5: −

### Compound 112

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,18Q,22V,23E,24I,28Y,30W,31L]hPYY(4-36)

iVal-APEK(C18DA-gGlu-OEG1-OEG2-)
PEEDATPEELQRYYVSLRHYYNWLTRQRY-NH2 (sequence
and structure below disclosed as SEQ ID NO: 116)

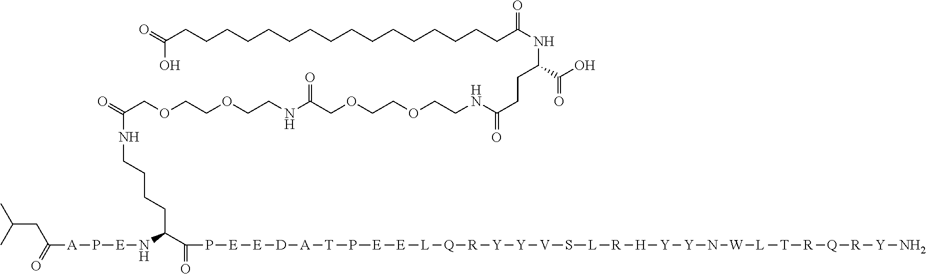

MW (calculated): 5001.6 Da

Synthesis and purification methods: S01; P01

LCMS: A01; Rt: 9.85 min; m/3: − m/4: 1252.0 m/5: −

### Compound 113

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,18Q,22V,23E,24I,28Y,30W,31L]hPYY(4-36)

(SEQ ID NO: 117)
iVal-APEK(C18DA-gGlu-OEG1-OEG2-)
AEEDATPEELQRYYVSLRHYYNWLTRQRY-NH2

MW (calculated): 4975.6 Da

Synthesis and purification methods: S01; P01

LCMS: A01; Rt: 10.22 min; m/3: 1659.0 m/4: − m/5: −

### Compound 114

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,18Q,22V,28H,29W,30Y,31L]hPYY(4-36)

(SEQ ID NO: 118)
iVal-APEK(C18DA-gGlu-OEG1-OEG2-)
PEEDASPEELQRYYVSLRHYHWYLTRQRY-NH2

MW (calculated): 5010.6 Da

Synthesis and purification methods: S02; P02

LCMS: A02; Rt: 12.38 min; m/3: 1671.2 m/4: 1253.9 m/5: −

### Compound 115

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,16A,18Q,22V,28Y,30W,31L]hPYY(4-36)

(SEQ ID NO: 119)
iVal-APEK(C18DA-gGlu-OEG1-OEG2-)
PEEEASPEALQRYYVSLRHYYNWLTRQRY-NH2

MW (calculated): 4943.6 Da

Synthesis and purification methods: S01; P01

LCMS: A01; Rt: 10.09 min; m/3: − m/4: 1237.0 m/5: −

### Compound 116

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,13A,14A,18Q,22V,28Y,30W,31L]hPYY(4-36)

(SEQ ID NO: 120)
iVal-APEK(C18DA-gGlu-OEG1-OEG2-)
PEEDAAAEELQRYYVSLRHYYNWLTRQRY-NH2

MW (calculated): 0.0 Da

Synthesis and purification methods: S01; P01

LCMS: A01; Rt: 11.36 min; m/3: − m/4: 1238.0 m/5: −

### Compound 117

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,6A,7K,9E,18Q,22V,23A,28Y,30W,31L]hPYY(4-36)

iVal-APAK(C18DA-gGlu-OEG1-OEG2-)
PEEDASPEELQRYYVALRHYYNWLTRQRY-NH2(sequence
and structure below disclosed as SEQ ID NO: 121)

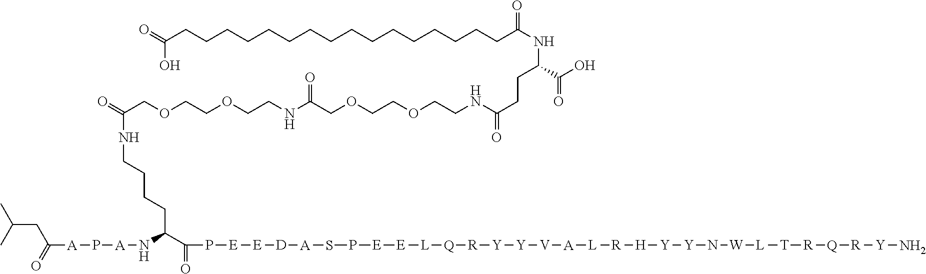

MW (calculated): 4913.6 Da

Synthesis and purification methods: S01; P01

LCMS: A01; Rt: 10.08 min; m/3: − m/4: 1228.0 m/5: −

### Compound 118

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,14A,15A,18Q,22V,28Y,30W,31L]hPYY(4-36)

(SEQ ID NO: 122)
iVal-APEK(C18DA-gGlu-OEG1-OEG2-)
PEEDASAAELQRYYVSLRHYYNWLTRQRY-NH2

MW (calculated): 4903.5 Da

Synthesis and purification methods: S01; P01

LCMS: A01; Rt: 10.49 min; m/3: 1636.0 m/4: − m/5: −

### Compound 119

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,14A,18Q,22V,28Y,30W,31L]hPYY(4-36)

(SEQ ID NO: 123)
iVal-APEK(C18DA-gGlu-OEG1-OEG2-)
PEEEASPEELQRYYVTLRHYYNWLTRQRY-NH2

MW (calculated): 5015.7 Da

Synthesis and purification methods: S01; P01

LCMS: A01; Rt: 9.99 min; m/3: − m/4: 1254.0 m/5: −

### Compound 120

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,13E,14E,18Q,19E,22V,28Y,30W,31L]hPYY(4-36)

(SEQ ID NO: 124)
iVal-APEK(C18DA-gGlu-OEG1-OEG2-)
PGEDAEEEELQEYYVSLRHYYNWLTRQRY-NH2

MW (calculated): 4962.5 Da

Synthesis and purification methods: S01; P01

LCMS: A01; Rt: 10.78 min; m/3: − m/4: 1241.0 m/5: −

### Compound 121

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,6A,7K,9E,10A,18Q,22V,28Y,30W,31L]hPYY(4-36)

(SEQ ID NO: 125)
iVal-APAK(C18DA-gGlu-OEG1-OEG2-)
PEADASPEELQRYYVSLRHYYNWLTRQRY-NH2

MW (calculated): 4871.5 Da

Synthesis and purification methods: S01; P01

LCMS: A01; Rt: 9.95 min; m/3: 1624.0 m/4: m/5: −

### Compound 122

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,13A,18Q,22V,28Y,30W,31L]hPYY(4-36)

(SEQ ID NO: 126)
iVal-APEK(C18DA-gGlu-OEG1-OEG2-)
PEEDAAPEELQRYYVSLRHYYNWLTRQRY-NH2

MW (calculated): 4971.6 Da

Synthesis and purification methods: S01; P01

LCMS: A01; Rt: 10.55 min; m/3: 1658.0 m/4: − m/5: −

### Compound 123

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,18Q,22V,28W,29Y,30H,31L]hPYY(4-36)

(SEQ ID NO: 127)
iVal-APEK(C18DA-gGlu-OEG1-OEG2-)
PEEDASPEELQRYYVSLRHYWYHLTRQRY-NH2

MW (calculated): 5010.6 Da

Synthesis and purification methods: S02; P02

LCMS: A02; Rt: 9.68 min; m/3: 1671.7 m/4: 1253.7 m/5: −

### Compound 124

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,13A,17I,18Q,22V,28Y,30W,31L]hPYY(4-36)

(SEQ ID NO: 128)
iVal-APEK(C18DA-gGlu-OEG1-OEG2-)
PEEDAAPEEIQRYYVSLRHYYNWLTRQRY-NH2

MW (calculated): 4971.6 Da

Synthesis and purification methods: S01; P01

LCMS: A01; Rt: 10.55 min; m/3: − m/4: 1244.0 m/5: −

### Compound 125

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,18Q,22V,28Y,30W,31A]hPYY(4-36)

(SEQ ID NO: 129)
iVal-APEK(C18DA-gGlu-OEG1-OEG2-)
PGEDASPEELQRYYVSLRHYYNWATRQRY-NH2

MW (calculated): 4873.5 Da

Synthesis and purification methods: S01; P01

LCMS: A01; Rt: 9.06 min; m/3: 1625.0 m/4: − m/5: −

### Compound 126

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,6A,7K,9E,12S,18Q,22V,28Y,30W,31L]hPYY(4-36)

(SEQ ID NO: 130)
iVal-APAK(C18DA-gGlu-OEG1-OEG2-)
PEEDSSPEELQRYYVSLRHYYNWLTRQRY-NH2

MW (calculated): 4945.6 Da

Synthesis and purification methods: S01; P01

LCMS: A01; Rt: 9.89 min; m/3: 1649.0 m/4: − m/5: −

### Compound 127

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,6A,7K,9E,14A,18Q,22V,28Y,30W,31L]hPYY(4-36)

(SEQ ID NO: 131)
iVal-APAK(C18DA-gGlu-OEG1-OEG2-)
PEEDASAEELQRYYVSLRHYYNWLTRQRY-NH2

MW (calculated): 4903.5 Da

Synthesis and purification methods: S01; P01

LCMS: A01; Rt: 10.80 min; m/3: − m/4: 1226.0 m/5: −

### Compound 128

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,13P,14A,17I,18Q,22V,28Y,30W,31L]hPYY(4-36)

(SEQ ID NO: 132)
iVal-APEK(C18DA-gGlu-OEG1-OEG2-)
PEEDAPAEEIQRYYVSLRHYYNWLTRQRY-NH2

MW (calculated): 4971.6 Da

Synthesis and purification methods: S01; P01

LCMS: A01; Rt: 11.28 min; m/3: 1658.0 m/4: − m/5: −

### Compound 129

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,14A,18Q,22V,28Y,30W,31L]hPYY(4-36)

(SEQ ID NO: 133)
iVal-APEK(C18DA-gGlu-OEG1-OEG2-)
PEEDASPEETQRYYTALRHYYNWLTRQRY-NH2

MW (calculated): 4961.5 Da

Synthesis and purification methods: S01; P01

LCMS: A01; Rt: 9.22 min; m/3: − m/4: 1241.0 m/5: −

### Compound 130

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,18Q,22V,23E,24I,28Y,30W,31L]hPYY(4-36)

(SEQ ID NO: 134)
iVal-APEK(C18DA-gGlu-OEG1-OEG2-)
PEEDTSAEELQRYYVSLRHYYNWLTRQRY-NH2

MW (calculated): 4991.6 Da

Synthesis and purification methods: S01; P01

LCMS: A01; Rt: 10.30 min; m/3: 1665.0 m/4: − m/5: −

### Compound 131

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,8A,9E,18Q,22V,23E,28Y,30W,31L]hPYY(4-36)

(SEQ ID NO: 135)
iVal-APEK(C18DA-gGlu-OEG1-OEG2-)
AEEDASPEELQRYYVELRHYYNWLTRQRY-NH2

MW (calculated): 5002.6 Da

Synthesis and purification methods: S01; P01

LCMS: A01; Rt: 10.10 min; m/3: − m/4: 1251.0 m/5: −

### Compound 132

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,14A,18Q,22V,28Y,30W,31L]hPYY(4-36)

(SEQ ID NO: 136)
iVal-APEK(C18DA-gGlu-OEG1-OEG2-)
PEEDASPEELQKYYVTLRHYYNWLTRQRY-NH2

MW (calculated): 4973.6 Da

Synthesis and purification methods: S03; P02

LCMS: A02; Rt: 11.78 min; m/3: 1659.4 m/4: 1244.6 m/5: 995.3

### Compound 133

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,17V,18Q,22V,23E,28Y,30W,31L]hPYY(4-36)

(SEQ ID NO: 137)
iVal-APEK(C18DA-gGlu-OEG1-OEG2-)
PEEDASPEEVQRYYVELRHYYNWLTRQRY-NH2

MW (calculated): 5015.6 Da

Synthesis and purification methods: S01; P01

LCMS: A01; Rt: 9.45 min; m/3: − m/4: 1254.0 m/5: −

### Compound 134

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,14A,18Q,22V,28Y,30W,31L]hPYY(4-36)

(SEQ ID NO: 138)
iVal-APEK(C18DA-gGlu-OEG1-OEG2-)
PEEDASPEEIQRYYTELRHYYNWLTRQRY-NH2

MW (calculated): 5031.6 Da

Synthesis and purification methods: S01; P01

LCMS: A01; Rt: 9.54 min; m/3: − m/4: 1258.0 m/5: −

### Compound 135

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,10A,18Q,22V,23E,28Y,30W,31L]hPYY(4-36)

iVal-APEK(C18DA-gGlu-OEG1-OEG2-)
PEADASPEELQRYYVELRHYYNWLTRQRY-NH2 (sequence
and structure below disclosed as SEQ ID NO: 139)

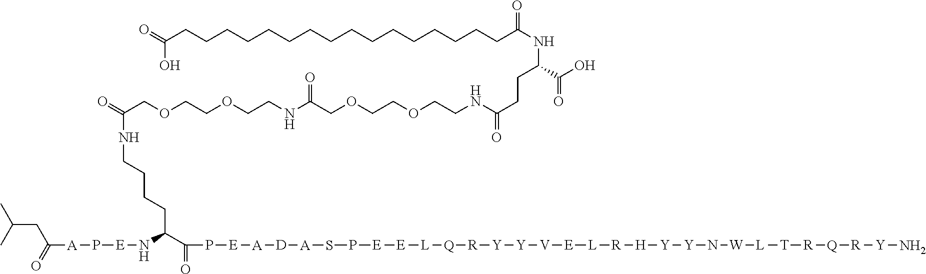

MW (calculated): 4971.6 Da

Synthesis and purification methods: S01; P01

LCMS: A01; Rt: 9.81 min; m/3: − m/4: 1244.0 m/5: −

### Compound 136

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,10A,12S,18Q,22V,28Y,30W,31L]hPYY(4-36)

(SEQ ID NO: 140)
iVal-APEK(C18DA-gGlu-OEG1-OEG2-)
PEADSSPEELQRYYVSLRHYYNWLTRQRY-NH2

MW (calculated): 4945.6 Da

Synthesis and purification methods: S01; P01

LCMS: A01; Rt: 9.79 min; m/3: 1649.0 m/4: − m/5: −

### Compound 137

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,18Q,22V,28Y,30W,31I]hPYY(4-36)

(SEQ ID NO: 141)
iVal-APEK(C18DA-gGlu-OEG1-OEG2-)
PEEDASPEELQRYYVSLRHYYNWITRQRY-NH2

MW (calculated): 4987.6 Da

Synthesis and purification methods: S01; P01

LCMS: A01; Rt: 9.56 min; m/3: 1664.0 m/4: − m/5: −

### Compound 138

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,6A,7K,9E,18Q,19Q,22V,28Y,30W,31L]hPYY(4-36)

(SEQ ID NO: 142)
iVal-APAK(C18DA-gGlu-OEG1-OEG2-)
PEEDASPEELQQYYVSLRHYYNWLTRQRY-NH2

MW (calculated): 4901.5 Da

Synthesis and purification methods: S01; P01

LCMS: A01; Rt: 10.03 min; m/3: − m/4: 1226.0 m/5: −

### Compound 139

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,12S,18Q,22V,28Y,30W,31L]hPYY(4-36)

(SEQ ID NO: 143)
iVal-APEK(C18DA-gGlu-OEG1-OEG2-)
PEEESSPEELQRYYVSLRHYYNWLTRQRY-NH2

MW (calculated): 5017.6 Da

Synthesis and purification methods: S01; P01

LCMS: A01; Rt: 9.79 min; m/3: − m/4: 1256.0 m/5: −

### Compound 140

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,18E,19Q,22V,28Y,30W,31L]hPYY(4-36)

(SEQ ID NO: 144)
iVal-APEK(C18DA-gGlu-OEG1-OEG2-)
PGEDASPEELEQYYVSLRHYYNWLTRQRY-NH2

MW (calculated): 4888.5 Da

Synthesis and purification methods: S01; P01

LCMS: A01; Rt: 10.01 min; m/3: − m/4: 1223.0 m/5: −

### Compound 141

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11A,18Q,22V,24A,28Y,30W,31L]hPYY(4-36)

(SEQ ID NO: 145)
iVal-APEK(C18DA-gGlu-OEG1-OEG2-)
PEEAASPEELQRYYVSARHYYNWLTRQRY-NH2

MW (calculated): 4901.5 Da

Synthesis and purification methods: S01; P01

LCMS: A01; Rt: 9.46 min; m/3: − m/4: 1227.0 m/5: −

### Compound 142

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9P,11E,18Q,22V,28Y,30W,31L]hPYY(4-36)

(SEQ ID NO: 146)
iVal-APEK(C18DA-gGlu-OEG1-OEG2-)
PPEEASPEELQRYYVSLRHYYNWLTRQRY-NH2

MW (calculated): 4969.6 Da

Synthesis and purification methods: S01; P01

LCMS: A01; Rt: 9.79 min; m/3: − m/4: 1243.0 m/5: −

### Compound 143

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,14A,18Q,22V,28Y,30W,31L]hPYY(4-36)

(SEQ ID NO: 147)
iVal-APEK(C18DA-gGlu-OEG1-OEG2-)
PEEDATPEELQRYYVALRHYYNWLTRQRY-NH2

MW (calculated): 4985.6 Da

Synthesis and purification methods: S01; P01

LCMS: A01; Rt: 10.10 min; m/3: 1662.0 m/4: − m/5: −

### Compound 144

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,8A,9E,18Q,19Q,22V,28Y,30W,31L]hPYY(4-36)

(SEQ ID NO: 148)
iVal-APEK(C18DA-gGlu-OEG1-OEG2-)
AEEDASPEELQQYYVSLRHYYNWLTRQRY-NH2

MW (calculated): 4933.5 Da

Synthesis and purification methods: S01; P01

LCMS: A01; Rt: 10.14 min; m/3: 1645.0 m/4: − m/5: −

### Compound 145

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,12S,17I,18Q,22V,28Y,30W,31L]hPYY(4-36)

(SEQ ID NO: 149)
iVal-APEK(C18DA-gGlu-OEG1-OEG2-)
PEEDSSPEEIQRYYVSLRHYYNWLTRQRY-NH2

MW (calculated): 5003.6 Da

Synthesis and purification methods: S01; P01

LCMS: A01; Rt: 9.74 min; m/3: − m/4: 1251.0 m/5: −

### Compound 146

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,18Q,22V,23E,24I,28Y,30W,31L]hPYY(4-36)

(SEQ ID NO: 150)
iVal-APEK(C18DA-gGlu-OEG1-OEG2-)
PEEEASPEELQRYYVSTRHYYNWLTRQRY-NH2

MW (calculated): 4989.6 Da

Synthesis and purification methods: S01; P01

LCMS: A01; Rt: 9.22 min; m/3: − m/4: 1248.0 m/5: −

### Compound 147

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,14A,18Q,22V,28Y,30W,31L]hPYY(4-36)

(SEQ ID NO: 151)
iVal-APEK(C18DA-gGlu-OEG1-OEG2-)
PEEDASPEETQKYYVSLRHYYNWLTRQRY-NH2

MW (calculated): 4947.5 Da

Synthesis and purification methods: S03; P02

LCMS: A02; Rt: 9.18 min; m/3: 1650.3 m/4: 1238.1 m/5: 990.4

### Compound 148

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,13P,14A,18Q,22V,28Y,30W,31L]hPYY(4-36)

(SEQ ID NO: 152)
iVal-APEK(C18DA-gGlu-OEG1-OEG2-)
PEEDAPAEELQRYYVSLRHYYNWLTRQRY-NH2

MW (calculated): 4971.6 Da

Synthesis and purification methods: S01; P01

LCMS: A01; Rt: 11.20 min; m/3: − m/4: 1242.0 m/5: −

### Compound 149

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11A,18Q,22V,23E,28Y,30W,31L]hPYY(4-36)

(SEQ ID NO: 153)
iVal-APEK(C18DA-gGlu-OEG1-OEG2-)
PEEAASPEELQRYYVELRHYYNWLTRQRY-NH2

MW (calculated): 4985.6 Da

Synthesis and purification methods: S01; P01

LCMS: A01; Rt: 9.99 min; m/3: − m/4: 1248.0 m/5: −

### Compound 150

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,13E,18Q,19K,22V,28Y,30W,31L]hPYY(4-36)

(SEQ ID NO: 154)
iVal-APEK(C18DA-gGlu-OEG1-OEG2-)
PEEDAEPEELQKYYVSLRHYYNWLTRQRY-NH2

MW (calculated): 5001.6 Da

Synthesis and purification methods: S03; P02

LCMS: A02; Rt: 11.23 min; m/3: 1668.7 m/4: 1251.5 m/5: 1001.2

### Compound 151

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,10A,11E,18Q,22V,28Y,30W,31L]hPYY(4-36)

(SEQ ID NO: 155)
iVal-APEK(C18DA-gGlu-OEG1-OEG2-)
PEAEASPEELQRYYVSLRHYYNWLTRQRY-NH2

MW (calculated): 4943.6 Da

Synthesis and purification methods: S01; P01

LCMS: A01; Rt: 9.87 min; m/3: − m/4: 1237.0 m/5: −

### Compound 152

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,8A,9E,17I,18Q,22V,28Y,30W,31L]hPYY(4-36)

(SEQ ID NO: 156)
iVal-APEK(C18DA-gGlu-OEG1-OEG2-)
AEEDASPEEIQRYYVSLRHYYNWLTRQRY-NH2

MW (calculated): 4961.6 Da

Synthesis and purification methods: S01; P01

LCMS: A01; Rt: 9.95 min; m/3: 1654.0 m/4: − m/5: −

### Compound 153

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,8V,9E,11E,18Q,22V,28Y,30W,31L]hPYY(4-36)

(SEQ ID NO: 157)
iVal-APEK(C18DA-gGlu-OEG1-OEG2-)
VEEEASPEELQRYYVSLRHYYNWLTRQRY-NH2

MW (calculated): 5003.7 Da

Synthesis and purification methods: S01; P01

LCMS: A01; Rt: 10.62 min; m/3: − m/4: − m/5: 1001

### Compound 154

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,18Q,21E,22V,28Y,30W,31L]hPYY(4-36)

(SEQ ID NO: 158)
iVal-APEK(C18DA-gGlu-OEG1-OEG2-)
PEEEASPEELQRYEVSLRHYYNWLTRQRY-NH2

MW (calculated): 4967.6 Da

Synthesis and purification methods: S03; P02

LCMS: A02; Rt: 10.32 min; m/3: 1657.2 m/4: 1243.0 m/5: 994.4

### Compound 155

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,14A,18Q,22V,28Y,30W,31L]hPYY(4-36)

(SEQ ID NO: 159)
iVal-APEK(C18DA-gGlu-OEG1-OEG2-)
PEEDASPEETQRYYVSLRHYYNWLTRQRY-NH2

MW (calculated): 4975.6 Da

Synthesis and purification methods: S01; P01

LCMS: A01; Rt: 9.19 min; m/3: 1659.0 m/4: − m/5: −

### Compound 156

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,14E,18E,19Q,22V,28Y,30W,31L]hPYY(4-36)

(SEQ ID NO: 160)
iVal-APEK(C18DA-gGlu-OEG1-OEG2-)
PEEDASEEELEQYYVSLRHYYNWLTRQRY-NH2

MW (calculated): 4992.5 Da

Synthesis and purification methods: S02; P02

LCMS: A02; Rt: 13.23 min; m/3: 1665.6 m/4: 1248.9 m/5: 999.2

### Compound 157

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,14A,18Q,22V,28Y,30W,31L]hPYY(4-36)

(SEQ ID NO: 161)
iVal-APAK(C18DA-gGlu-OEG1-OEG2-)
PEEDASPEETQRYYVSLRHYYNWLTRQRY-NH2

MW (calculated): 4917.5 Da

Synthesis and purification methods: S01; P01

LCMS: A01; Rt: 9.28 min; m/3: 1640.0 m/4: − m/5: −

### Compound 158

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,18Q,22V,28W,30Y,31L]hPYY(4-36)

(SEQ ID NO: 162)
iVal-APEK(C18DA-gGlu-OEG1-OEG2-)
PEEDASPEELQRYYVSLRHYWNYLTRQRY-NH2

MW (calculated): 4987.6 Da

Synthesis and purification methods: S01; P01

LCMS: A01; Rt: 10.09 min; m/3: − m/4: 1248.0 m/5: −

### Compound 159

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,10A,18E,19Q,22V,28Y,30W,31L]hPYY(4-36)

(SEQ ID NO: 163)
iVal-APEK(C18DA-gGlu-OEG1-OEG2-)
PEADASPEELEQYYVSLRHYYNWLTRQRY-NH2

MW (calculated): 4902.5 Da

Synthesis and purification methods: S01; P01

LCMS: A01; Rt: 10.13 min; m/3: − m/4: 1226.0 m/5: −

### Compound 160

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,18Q,22V,23E,24I,28Y,30W,31L]hPYY(4-36)

iVal-APEK(C18DA-gGlu-OEG1-OEG2-)
PEADAEPEELQRYYTSLRHYYNWLTRQRY-NH2
(sequence and structure below
disclosed as SEQ ID NO: 164)

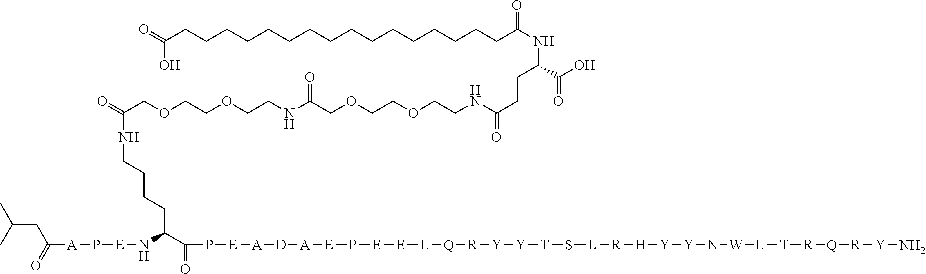

MW (calculated): 4973.6 Da

Synthesis and purification methods: S01; P01

LCMS: A01; Rt: 10.10 min; m/3: − m/4: 1244.0 m/5: −

### Compound 161

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,14A,18Q,22V,28Y,30W,31L]hPYY(4-36)

iVal-APEK(C18DA-gGlu-OEG1-OEG2-)
PEEDATPEELQQYYVSLRHYYNWLTRQRY-NH2
(sequence and structure below
disclosed as SEQ ID NO: 165)

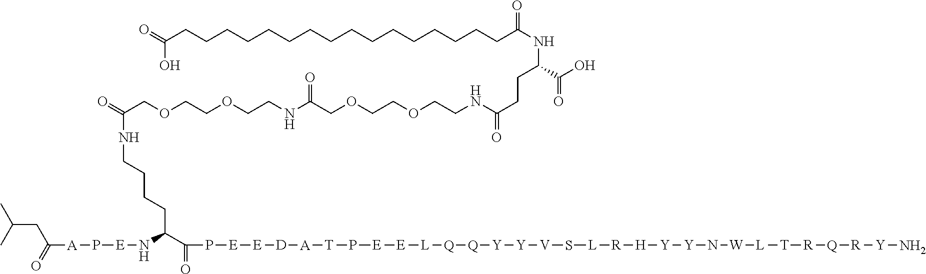

MW (calculated): 4973.6 Da

Synthesis and purification methods: S03; P02

LCMS: A02; Rt: 11.90 min; m/3: 1658.2 m/4: 1244.5 m/5: 995.7

### Compound 162

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,18Q,21E,22V,26A,28Y,30W,31L]hPYY(4-36)

(SEQ ID NO: 166)
iVal-APEK(C18DA-gGlu-OEG1-OEG2-)
PEEDASPEELQRYEVSLRAYYNWLTRQRY-NH2

MW (calculated): 4887.5 Da

Synthesis and purification methods: S03; P02

LCMS: A02; Rt: 11.22 min; m/3: 1630.5 m/4: 1223.0 m/5: 978.3

### Compound 163

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11A,18Q,22V,23A,28Y,30W,31L]hPYY(4-36)

(SEQ ID NO: 167)
iVal-APEK(C18DA-gGlu-OEG1-OEG2-)
PEEAASPEELQRYYVALRHYYNWLTRQRY-NH2

MW (calculated): 4927.6 Da

Synthesis and purification methods: S01; P01

LCMS: A01; Rt: 10.16 min; m/3: − m/4: 1234.0 m/5: −

### Compound 164

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,14E,18Q,19E,22V,28Y,30W,31L]hPYY(4-36)

(SEQ ID NO: 168)
iVal-APEK(C18DA-gGlu-OEG1-OEG2-)
PEEDASEEELQEYYVSLRHYYNWLTRQRY-NH2

MW (calculated): 4992.5 Da

Synthesis and purification methods: S01; P01

LCMS: A01; Rt: 10.65 min; m/3: 1665.0 m/4: − m/5: −

### Compound 165

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,18Q,21Q,22V,28Y,30W,31L]hPYY(4-36)

(SEQ ID NO: 169)
iVal-APEK(C18DA-gGlu-OEG1-OEG2-)
PEEDASPEELQRYQVSLRHYYNWLTRQRY-NH2

MW (calculated): 4952.6 Da

Synthesis and purification methods: S01; P01

LCMS: A01; Rt: 9.26 min; m/3: − m/4: 1239.0 m/5: −

### Compound 166

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9P,10A,18Q,22V,23E,28Y,30W,31L]hPYY(4-36)

(SEQ ID NO: 170)
iVal-APEK(C18DA-gGlu-OEG1-OEG2-)
PPADASPEELQRYYVELRHYYNWLTRQRY-NH2

MW (calculated): 4939.6 Da

Synthesis and purification methods: S01; P01

LCMS: A01; Rt: 9.58 min; m/3: 1647.0 m/4: − m/5: −

### Compound 167

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,18Q,19Q,21E,22V,28Y,30W,31L]hPYY(4-36)

(SEQ ID NO: 171)
iVal-APEK(C18DA-gGlu-OEG1-OEG2-)
PEEDASPEELQQYEVSLRHYYNWLTRQRY-NH2

MW (calculated): 4925.5 Da

Synthesis and purification methods: S03; P02

LCMS: A02; Rt: 10.95 min; m/3: 1643.1 m/4: 1232.5 m/5: 985.5

### Compound 168

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,13E,18Q,22V,28Y,30W,31I]hPYY(4-36)

(SEQ ID NO: 172)
iVal-APEK(C18DA-gGlu-OEG1-OEG2-)
PEEDAEPEELQRYYVSLRHYYNWITRQRY-NH2

MW (calculated): 5029.6 Da

Synthesis and purification methods: S01; P01

LCMS: A01; Rt: 9.66 min; m/3: 1678.0 m/4: − m/5: −

### Compound 169

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,18Q,22V,23A,28Y,30W,31L]hPYY(4-36)

(SEQ ID NO: 173)
iVal-APEK(C18DA-gGlu-OEG1-OEG2-)
PEEEASPEELQRYYVALRHYYNWLTRQRY-NH2

MW (calculated): 4985.6 Da

Synthesis and purification methods: S01; P01

LCMS: A01; Rt: 9.96 min; m/3: 1663.0 m/4: − m/5: −

### Compound 170

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,18E,19Q,22V,28Y,30W,31L]hPYY(4-36)

(SEQ ID NO: 174)
iVal-APEK(C18DA-gGlu-OEG1-OEG2-)
PEEEASPEELEQYYVSLRHYYNWLTRQRY-NH2

MW (calculated): 4974.6 Da

Synthesis and purification methods: S01; P01

LCMS: A01; Rt: 10.18 min; m/3: − m/4: 1244.0 m/5: −

### Compound 171

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,14A,18Q,22V,28Y,30W,31L]hPYY(4-36)

iVal-APEK(C18DA-gGlu-OEG1-OEG2-)
PEEDATPEEIQRYYVSLRHYYNWLTRQRY-NH2
(sequence and structure below
disclosed as SEQ ID NO: 175)

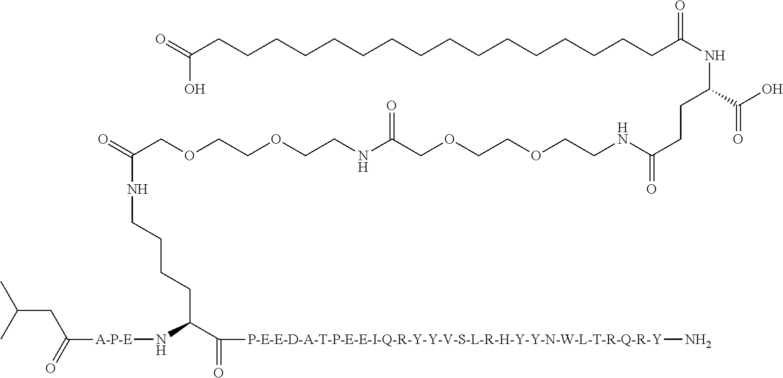

MW (calculated): 5001.6 Da

Synthesis and purification methods: S01; P01

LCMS: A01; Rt: 9.82 min; m/3: − m/4: 1251.0 m/5: −

### Compound 172

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,13E,18Q,22V,23A,28Y,30W,31L]hPYY(4-36)

(SEQ ID NO: 176)
iVal-APEK(C18DA-gGlu-OEG1-OEG2-)
PEEDAEPEELQRYYVALRHYYNWLTRQRY-NH2

MW (calculated): 5013.6 Da

Synthesis and purification methods: S03; P02

LCMS: A02; Rt: 12.87 min; m/3: 1672.6 m/4: 1254.6 m/5: 1003.6

### Compound 173

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,6A,7K,9E,18Q,22V,23E,28Y,30W,31L]hPYY(4-36)

(SEQ ID NO: 177)
iVal-APAK(C18DA-gGlu-OEG1-OEG2-)
PEEDASPEELQRYYVELRHYYNWLTRQRY-NH2

MW (calculated): 4971.6 Da

Synthesis and purification methods: S01; P01

LCMS: A01; Rt: 9.91 min; m/3: − m/4: 1245.0 m/5: −

### Compound 174

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,6A,7K,9E,13E,18Q,22V,28Y,30W,31L]hPYY(4-36)

(SEQ ID NO: 178)
iVal-APAK(C18DA-gGlu-OEG1-OEG2-)
PEEDAEPEELQRYYVSLRHYYNWLTRQRY-NH2

MW (calculated): 4971.6 Da

Synthesis and purification methods: S01; P01

LCMS: A01; Rt: 10.27 min; m/3: − m/4: 1244.0 m/5: −

### Compound 175

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,13A,18Q,22V,23A,28Y,30W,31L]hPYY(4-36)

(SEQ ID NO: 179)
iVal-APEK(C18DA-gGlu-OEG1-OEG2-)
PEEDAAPEELQRYYVALRHYYNWLTRQRY-NH2

MW (calculated): 4955.6 Da

Synthesis and purification methods: S01; P01

LCMS: A01; Rt: 10.87 min; m/3: 1653.0 m/4: − m/5: −

### Compound 176

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,14E,18Q,22V,28Y,30W,31L]hPYY(4-36)

(SEQ ID NO: 180)
iVal-APEK(C18DA-gGlu-OEG1-OEG2-)
PEEDASEEELQRYYVSLRHYYNWLTRQRY-NH2

MW (calculated): 5019.6 Da

Synthesis and purification methods: S01; P01

LCMS: A01; Rt: 10.40 min; m/3: 1674.0 m/4: − m/5: −

### Compound 177

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,12S,18Q,22V,23E,28Y,30W,31L]hPYY(4-36)

(SEQ ID NO: 181)
iVal-APEK(C18DA-gGlu-OEG1-OEG2-)
PEEDSSPEELQRYYVELRHYYNWLTRQRY-NH2

MW (calculated): 5045.6 Da

Synthesis and purification methods: S01; P01

LCMS: A01; Rt: 9.85 min; m/3: − m/4: 1262.0 m/5: −

### Compound 178

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,18Q,22V,23E,24I,28Y,30W,31L]hPYY(4-36)

(SEQ ID NO: 182)
iVal-APEK(C18DA-gGlu-OEG1-OEG2-)
PEEDATPEETERYYVSLRHYYNWLTRQRY-NH2

MW (calculated): 4990.6 Da

Synthesis and purification methods: S01; P01

LCMS: A01; Rt: 9.35 min; m/3: − m/4: 1249.0 m/5: −

### Compound 179

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,6A,7K,9E,15A,18Q,22V,28Y,30W,31L]hPYY(4-36)

(SEQ ID NO: 183)
iVal-APAK(C18DA-gGlu-OEG1-OEG2-)
PEEDASPAELQRYYVSLRHYYNWLTRQRY-NH2

MW (calculated): 4871.5 Da

Synthesis and purification methods: S01; P01

LCMS: A01; Rt: 9.96 min; m/3: 1625.0 m/4: m/5: −

### Compound 180

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,14A,18Q,22V,28Y,30W,31L]hPYY(4-36)

(SEQ ID NO: 184)
iVal-APEK(C18DA-gGlu-OEG1-OEG2-)
PEEDASPEETQQYYVSLRHYYNWLTRQRY-NH2

MW (calculated): 4947.5 Da

Synthesis and purification methods: S01; P01

LCMS: A01; Rt: 9.27 min; m/3: 1650.0 m/4: − m/5: −

### Compound 181

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,10A,18Q,22V,23A,28Y,30W,31L]hPYY(4-36)

(SEQ ID NO: 185)
iVal-APEK(C18DA-gGlu-OEG1-OEG2-)
PEADASPEELQRYYVALRHYYNWLTRQRY-NH2

MW (calculated): 4913.6 Da

Synthesis and purification methods: S01; P01

LCMS: A01; Rt: 9.93 min; m/3: − m/4: 1229.0 m/5: −

### Compound 182

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,12G,18Q,22V,23E,28Y,30W,31L]hPYY(4-36)

(SEQ ID NO: 186)
iVal-APEK(C18DA-gGlu-OEG1-OEG2-)
PEEDGSPEELQRYYVELRHYYNWLTRQRY-NH2

MW (calculated): 5015.6 Da

Synthesis and purification methods: S01; P01

LCMS: A01; Rt: 11.02 min; m/3: 1672.0 m/4: − m/5: −

### Compound 183

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9P,18Q,22V,28Y,30W,31L]hPYY(4-36)

(SEQ ID NO: 187)
iVal-APEK(C18DA-gGlu-OEG1-OEG2-)
PPEDASPEELQRYYVSLRHYYNWLTRQRY-NH2

MW (calculated): 4955.6 Da

Synthesis and purification methods: S01; P01

LCMS: A01; Rt: 9.46 min; m/3: 1652.0 m/4: − m/5: −

### Compound 184

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,14A,18Q,22V,28Y,30W,31L]hPYY(4-36)

(SEQ ID NO: 188)
iVal-APEK(C18DA-gGlu-OEG1-OEG2-)
PEEAASPEELQRYYVTLRHYYNWLTRQRY-NH2

MW (calculated): 4957.6 Da

Synthesis and purification methods: S01; P01

LCMS: A01; Rt: 10.13 min; m/3: − m/4: 1241.0 m/5: −

### Compound 185

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,18Q,22V,23E,28Y,30W,31L]hPYY(4-36)

(SEQ ID NO: 189)
iVal-APEK(C18DA-gGlu-OEG1-OEG2-)
PEEEASPEELQRYYVELRHYYNWLTRQRY-NH2

MW (calculated): 5043.7 Da

Synthesis and purification methods: S01; P01

LCMS: A01; Rt: 9.83 min; m/3: 1683.0 m/4: − m/5: −

### Compound 186

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,14A,18Q,22V,23E,28Y,30W,31L]hPYY(4-36)

(SEQ ID NO: 190)
iVal-APEK(C18DA-gGlu-OEG1-OEG2-)
PEEDASAEELQRYYVELRHYYNWLTRQRY-NH2

MW (calculated): 5003.6 Da

Synthesis and purification methods: S01; P01

LCMS: A01; Rt: 10.55 min; m/3: − m/4: 1252.0 m/5: −

### Compound 187

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,18Q,22V,27Q,28Y,30W,31L]hPYY(4-36)

(SEQ ID NO: 191)
iVal-APEK(C18DA-gGlu-OEG1-OEG2-)
PEEDASPEELQRYYVSLRHQYNWLTRQRY-NH2

MW (calculated): 4952.6 Da

Synthesis and purification methods: S01; P01

LCMS: A01; Rt: 9.22 min; m/3: 1652.0 m/4: − m/5: −

### Compound 188

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,13E,18Q,22V,28Y,30W,31L]hPYY(4-36)

(SEQ ID NO: 192)
iVal-APEK(C18DA-gGlu-OEG1-OEG2-)
PEEDAEPEELQRYYVSLRHYYNWLTRQRY-NH2

MW (calculated): 5031.7 Da

Synthesis and purification methods: S01; P01

LCMS: A01; Rt: 9.87 min; m/3: − m/4: 1257.0 m/5: −

### Compound 189

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,14E,15A,18Q,22V,28Y,30W,31L]hPYY(4-36)

(SEQ ID NO: 193)
iVal-APEK(C18DA-gGlu-OEG1-OEG2-)
PEEDASEAELQRYYVSLRHYYNWLTRQRY-NH2

MW (calculated): 4961.6 Da

Synthesis and purification methods: S01; P01

LCMS: A01; Rt: 10.40 min; m/3: − m/4: 1241.0 m/5: −

### Compound 190

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,13P,18Q,22V,28Y,30W,31L]hPYY(4-36)

(SEQ ID NO: 194)
iVal-APEK(C18DA-gGlu-OEG1-OEG2-)
PEEEAPPEELQRYYVSLRHYYNWLTRQRY-NH2

MW (calculated): 5011.7 Da

Synthesis and purification methods: S01; P01

LCMS: A01; Rt: 10.32 min; m/3: − m/4: 1254.0 m/5: −

### Compound 191

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,18Q,22I,28Y,30W,31L]hPYY(4-36)

(SEQ ID NO: 195)
iVal-APEK(C18DA-gGlu-OEG1-OEG2-)
PEEDASPEELQRYYISLRHYYNWLTRQRY-NH2

MW (calculated): 5001.6 Da

Synthesis and purification methods: S01; P01

LCMS: A01; Rt: 9.81 min; m/3: 1668.0 m/4: − m/5: −

### Compound 192

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,13A,15A,18Q,22V,28Y,30W,31L]hPYY(4-36)

iVal-APEK(C18DA-gGlu-OEG1-OEG2-)
PEEDAAPAELQRYYVSLRHYYNWLTRQRY-NH2 (sequence
and structure below disclosed as SEQ ID NO: 196)

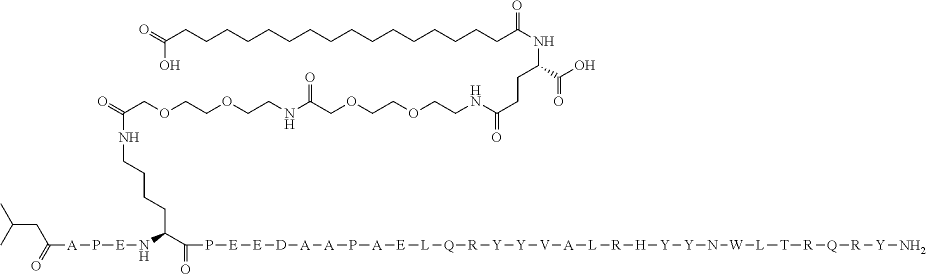

MW (calculated): 4913.6 Da

Synthesis and purification methods: S01; P01

LCMS: A01; Rt: 10.60 min; m/3: − m/4: 1229.0 m/5: −

### Compound 193

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,14E,17I,18Q,22V,28Y,30W,31L]hPYY(4-36)

(SEQ ID NO: 197)
iVal-APEK(C18DA-gGlu-OEG1-OEG2-)
PEEDASEEEIQRYYVSLRHYYNWLTRQRY-NH2

MW (calculated): 5019.6 Da

Synthesis and purification methods: S01; P01

LCMS: A01; Rt: 10.37 min; m/3: 1674.0 m/4: − m/5: −

### Compound 194

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,18Q,22V,23E,24I,28Y,30W,31L]hPYY(4-36)

(SEQ ID NO: 198)
iVal-AHypEK(C18DA-gGlu-OEG1-OEG2-)
PEEDASPEELQRYYVSLRHYYNWTLRQRY-NH2

MW (calculated): 5003.6 Da

Synthesis and purification methods: S01; P01

LCMS: A01; Rt: 8.84 min; m/3: − m/4: 1252.0 m/5: −

### Compound 195

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,16A,18Q,22V,23E,28Y,30W,31L]hPYY(4-36)

(SEQ ID NO: 199)
iVal-APEK(C18DA-gGlu-OEG1-OEG2-)
PEEDASPEALQRYYVELRHYYNWLTRQRY-NH2

MW (calculated): 4971.6 Da

Synthesis and purification methods: S01; P01

LCMS: A01; Rt: 10.11 min; m/3: 1658.0 m/4: − m/5: −

### Compound 196

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,6A,7K,9P,13E,18Q,22V,28Y,30W,31L]hPYY(4-36)

(SEQ ID NO: 200)
iVal-APAK(C18DA-gGlu-OEG1-OEG2-)
PPEDAEPEELQRYYVSLRHYYNWLTRQRY-NH2

MW (calculated): 4939.6 Da

Synthesis and purification methods: S01; P01

LCMS: A01; Rt: 10.28 min; m/3: − m/4: 1236.0 m/5: −

### Compound 197

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,6A,7K,9E,10A,18Q,22V,23E,28Y,30W,31L]hPYY(4-36)

(SEQ ID NO: 201)
iVal-APAK(C18DA-gGlu-OEG1-OEG2-)
PEADASPEELQRYYVELRHYYNWLTRQRY-NH2

MW (calculated): 4913.6 Da

Synthesis and purification methods: S01; P01

LCMS: A01; Rt: 10.00 min; m/3: 1639.0 m/4: m/5: −

### Compound 198

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,13E,16A,18Q,22V,28Y,30W,31L]hPYY(4-36)

(SEQ ID NO: 202)
iVal-APEK(C18DA-gGlu-OEG1-OEG2-)
PEEDAEPEALQRYYVSLRHYYNWLTRQRY-NH2

MW (calculated): 4971.6 Da

Synthesis and purification methods: S01; P01

LCMS: A01; Rt: 10.31 min; m/3: − m/4: 1244.0 m/5: −

### Compound 199

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,18Q,22V,23E,24I,28Y,30W,31L]hPYY(4-36)

(SEQ ID NO: 203)
iVal-APEK(C18DA-gGlu-OEG1-OEG2-)
PEEDAEPEETQRYYVSLRHYYNWLTRQRY-NH2

MW (calculated): 5017.6 Da

Synthesis and purification methods: S01; P01

LCMS: A01; Rt: 9.37 min; m/3: − m/4: 1255.0 m/5: −

### Compound 200

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,8Hyp,9E,18Q,22V,28Y,30W,31L]hPYY(4-36)

(SEQ ID NO: 204)
iVal-APEK(C18DA-gGlu-OEG1-OEG2-)
HypEEDASPEELQRYYVSLRHYYNWLTRQRY-NH2

MW (calculated): 5003.6 Da

Synthesis and purification methods: S01; P01

LCMS: A01; Rt: 9.53 min; m/3: − m/4: 1250.0 m/5: −

### Compound 201

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9Q,13E,18Q,19E,22V,28Y,30W,31L]hPYY(4-36)

(SEQ ID NO: 205)
iVal-APEK(C18DA-gGlu-OEG1-OEG2-)
PQEDAEPEELQEYYVSLRHYYNWLTRQRY-NH2

MW (calculated): 5001.6 Da

Synthesis and purification methods: S01; P01

LCMS: A01; Rt: 10.24 min; m/3: − m/4: 1251.0 m/5: −

### Compound 202

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,13A,18Q,19K,22V,28Y,30W,31L]hPYY(4-36)

(SEQ ID NO: 206)
iVal-APEK(C18DA-gGlu-OEG1-OEG2-)
PEEDAAPEELQKYYVSLRHYYNWLTRQRY-NH2

MW (calculated): 4943.6 Da

Synthesis and purification methods: S01; P01

LCMS: A01; Rt: 10.48 min; m/3: − m/4: 1237.0 m/5: −

### Compound 203

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,6A,7K,9E,18Q,22V,28Y,30W,31L]hPYY(4-36)

iVal-APAK(C18DA-gGlu-OEG1-OEG2-)
PEEDASPEELQRYYVSLRHYYNWLTRQRY-NH2 (sequence
and structure below disclosed as SEQ ID NO: 207)

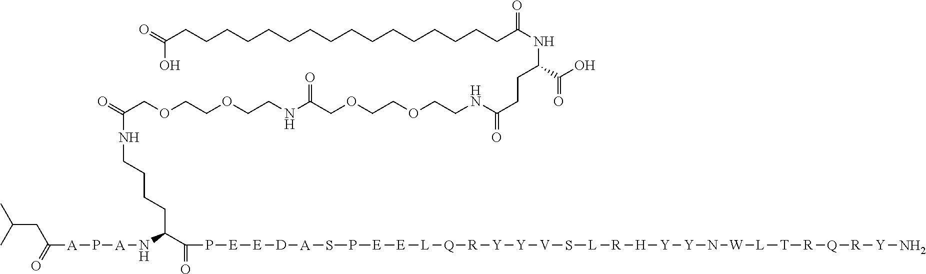

MW (calculated): 4929.6 Da

Synthesis and purification methods: S01; P01

LCMS: A01; Rt: 9.83 min; m/3: 1644.0 m/4: − m/5: −

### Compound 204

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,17V,18Q,22V,28Y,30W,31L]hPYY(4-36)

(SEQ ID NO: 208)
iVal-APEK(C18DA-gGlu-OEG1-OEG2-)
PEEEASPEEVQRYYVSLRHYYNWLTRQRY-NH2

MW (calculated): 4987.6 Da

Synthesis and purification methods: S01; P01

LCMS: A01; Rt: 9.62 min; m/3: 1663.0 m/4: − m/5: −

### Compound 205

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,18Q,19K,22V,28Y,30W,31L]hPYY(4-36)

(SEQ ID NO: 209)
iVal-APEK(C18DA-gGlu-OEG1-OEG2-)
PEEEASPEELQKYYVSLRHYYNWLTRQRY-NH2

MW (calculated): 4973.6 Da

Synthesis and purification methods: S03; P02

LCMS: A02; Rt: 11.05 min; m/3: 1659.2 m/4: 1244.4 m/5: 995.5

### Compound 206

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,18E,19Q,21E,22V,28Y,30W,31L]hPYY(4-36)

(SEQ ID NO: 210)
iVal-APEK(C18DA-gGlu-OEG1-OEG2-)
PEEDASPEELEQYEVSLRHYYNWLTRQRY-NH2

MW (calculated): 4926.5 Da

Synthesis and purification methods: S02; P02

LCMS: A02; Rt: 11.60 min; m/3: 1643.3 m/4: 1232.8 m/5: 986.2

### Compound 207

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,6A,7K,9E,10A,13E,18Q,22V,28Y,30W,31L]hPYY(4-36)

(SEQ ID NO: 211)
iVal-APAK(C18DA-gGlu-OEG1-OEG2-)
PEADAEPEELQRYYVSLRHYYNWLTRQRY-NH2

MW (calculated): 4913.6 Da

Synthesis and purification methods: S01; P01

LCMS: A01; Rt: 10.37 min; m/3: − m/4: 1229.0 m/5: −

### Compound 208

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,14A,18Q,22V,28Y,30W,31L]hPYY(4-36)

(SEQ ID NO: 212)
iVal-APEK(C18DA-gGlu-OEG1-OEG2-)
PEEDASEEETQRYYVSLRHYYNWLTRQRY-NH2

MW (calculated): 5007.6 Da

Synthesis and purification methods: S01; P01

LCMS: A01; Rt: 9.63 min; m/3: 1670.0 m/4: − m/5: −

### Compound 209

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,12S,13E,18Q,22V,28Y,30W,31L]hPYY(4-36)

(SEQ ID NO: 213)
iVal-APEK(C18DA-gGlu-OEG1-OEG2-)
PEEDSEPEELQRYYVSLRHYYNWLTRQRY-NH2

MW (calculated): 5045.6 Da

Synthesis and purification methods: S01; P01

LCMS: A01; Rt: 10.09 min; m/3: − m/4: 1263.0 m/5: −

### Compound 210

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,8I,9E,11E,18Q,22V,28Y,30W,31L]hPYY(4-36)

(SEQ ID NO: 214)
iVal-APEK(C18DA-gGlu-OEG1-OEG2-)
IEEEASPEELQRYYVSLRHYYNWLTRQRY-NH2

MW (calculated): 5017.7 Da

Synthesis and purification methods: S01; P01

LCMS: A01; Rt: 11.05 min; m/3: − m/4: 1255.0 m/5: −

### Compound 211

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,14A,18Q,22V,28Y,30W,31L]hPYY(4-36)

(SEQ ID NO: 215)
iVal-APEK(C18DA-gGlu-OEG1-OEG2-)
PEEDATAEELQRYYVSLRHYYNWLTRQRY-NH2

MW (calculated): 4975.6 Da

Synthesis and purification methods: S01; P01

LCMS: A01; Rt: 10.92 min; m/3: − m/4: 1245.0 m/5: −

### Compound 212

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,14A,18Q,22V,28Y,30W,31L]hPYY(4-36)

(SEQ ID NO: 216)
iVal-APEK(C18DA-gGlu-OEG1-OEG2-)
PEEDTAPEELQRYYVSLRHYYNWLTRQRY-NH2

MW (calculated): 5001.6 Da

Synthesis and purification methods: S01; P01

LCMS: A01; Rt: 10.56 min; m/3: − m/4: 1251.0 m/5: −

### Compound 213

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,14A,18Q,22V,28Y,30W,31L]hPYY(4-36)

(SEQ ID NO: 217)
iVal-APEK(C18DA-gGlu-OEG1-OEG2-)
PEEETSPEELQRYYVSLRHYYNWLTRQRY-NH2

MW (calculated): 5031.7 Da

Synthesis and purification methods: S01; P01

LCMS: A01; Rt: 9.79 min; m/3: 1678.0 m/4: − m/5: −

### Compound 214

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,14E,18Q,22V,23A,28Y,30W,31L]hPYY(4-36)

(SEQ ID NO: 218)
iVal-APEK(C18DA-gGlu-OEG1-OEG2-)
PEEDASEEELQRYYVALRHYYNWLTRQRY-NH2

MW (calculated): 5003.6 Da

Synthesis and purification methods: S01; P01

LCMS: A01; Rt: 10.70 min; m/3: 1668.0 m/4: − m/5: −

### Compound 215

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,15A,18Q,22V,23E,28Y,30W,31L]hPYY(4-36)

(SEQ ID NO: 219)
iVal-APEK(C18DA-gGlu-OEG1-OEG2-)
PEEDASPAELQRYYVELRHYYNWLTRQRY-NH2

MW (calculated): 4971.6 Da

Synthesis and purification methods: S01; P01

LCMS: A01; Rt: 9.86 min; m/3: − m/4: 1244.0 m/5: −

### Compound 216

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,6A,7K,9E,18E,19Q,22V,28Y,30W,31L]hPYY(4-36)

(SEQ ID NO: 220)
iVal-APAK(C18DA-gGlu-OEG1-OEG2-)
PEEDASPEELEQYYVSLRHYYNWLTRQRY-NH2

MW (calculated): 4902.5 Da

Synthesis and purification methods: S01; P01

LCMS: A01; Rt: 10.24 min; m/3: 1635.0 m/4: − m/5: −

### Compound 217

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,15A,18Q,22V,23A,28Y,30W,31L]hPYY(4-36)

(SEQ ID NO: 221)
iVal-APEK(C18DA-gGlu-OEG1-OEG2-)
PEEDASPAELQRYYVALRHYYNWLTRQRY-NH2

MW (calculated): 4913.6 Da

Synthesis and purification methods: S01; P01

LCMS: A01; Rt: 9.99 min; m/3: 1639.0 m/4: m/5: −

### Compound 218

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,12S,18Q,22V,23A,28Y,30W,31L]hPYY(4-36)

(SEQ ID NO: 222)
iVal-APEK(C18DA-gGlu-OEG1-OEG2-)
PEEDSSPEELQRYYVALRHYYNWLTRQRY-NH2

MW (calculated): 4987.6 Da

Synthesis and purification methods: S01; P01

LCMS: A01; Rt: 10.00 min; m/3: − m/4: 1247.0 m/5: −

### Compound 219

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,13A,18Q,22V,28Y,30W,31L]hPYY(4-36)

(SEQ ID NO: 223)
iVal-APEK(C18DA-gGlu-OEG1-OEG2-)
PEEEAAPEELQRYYVSLRHYYNWLTRQRY-NH2

MW (calculated): 4985.6 Da

Synthesis and purification methods: S01; P01

LCMS: A01; Rt: 10.41 min; m/3: − m/4: 1247.0 m/5: −

### Compound 220

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11A,14A,18Q,22V,28Y,30W,31L]hPYY(4-36)

(SEQ ID NO: 224)
iVal-APEK(C18DA-gGlu-OEG1-OEG2-)
PEEAASAEELQRYYVSLRHYYNWLTRQRY-NH2

MW (calculated): 4917.6 Da

Synthesis and purification methods: S01; P01

LCMS: A01; Rt: 10.70 min; m/3: − m/4: 1230.0 m/5: −

### Compound 221

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,13P,14E,18Q,22V,28Y,30W,31L]hPYY(4-36)

iVal-APEK(C18DA-gGlu-OEG1-OEG2-)
PEEDAPEEELQRYYVSLRHYYNWLTRQRY-NH2
(sequence and structure below
disclosed as SEQ ID NO: 225)

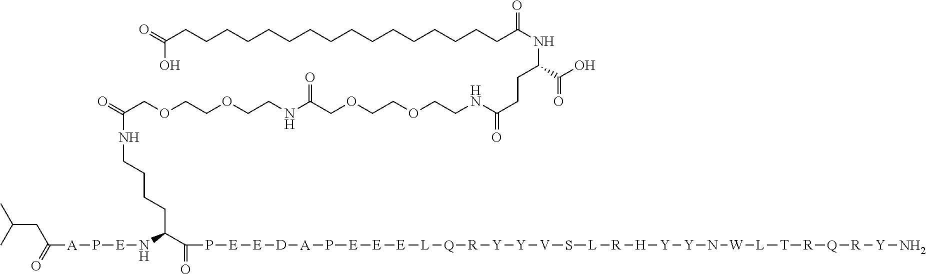

MW (calculated): 5029.6 Da

Synthesis and purification methods: S01; P01

LCMS: A01; Rt: 11.00 min; m/3: − m/4: 1258.0 m/5: −

### Compound 222

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,14A,18Q,19Q,22V,28Y,30W,31L]hPYY(4-36)

(SEQ ID NO: 226)
iVal-APEK(C18DA-gGlu-OEG1-OEG2-)
PEEDASAEELQQYYVSLRHYYNWLTRQRY-NH2

MW (calculated): 4933.5 Da

Synthesis and purification methods: S02; P02

LCMS: A02; Rt: 13.32 min; m/3: 1645.5 m/4: 1234.4 m/5: 987.4

### Compound 223

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11A,13E,18Q,22V,28Y,30W,31L]hPYY(4-36)

(SEQ ID NO: 227)
iVal-APEK(C18DA-gGlu-OEG1-OEG2-)
PEEAAEPEELQRYYVSLRHYYNWLTRQRY-NH2

MW (calculated): 4985.6 Da

Synthesis and purification methods: S01; P01

LCMS: A01; Rt: 10.21 min; m/3: − m/4: 1247.0 m/5: −

### Compound 224

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,11E,18E,19Q,22V,28Y,30W,31L]hPYY(4-36)

(SEQ ID NO: 228)
iVal-APEK(C18DA-gGlu-OEG1-OEG2-)
PGEEASPEELEQYYVSLRHYYNWLTRQRY-NH2

MW (calculated): 4902.5 Da

Synthesis and purification methods: S01; P01

LCMS: A01; Rt: 10.12 min; m/3: 1634.0 m/4: − m/5: −

### Compound 225

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,13E,18E,19Q,22V,28Y,30W,31L]hPYY(4-36)

(SEQ ID NO: 229)
iVal-APEK(C18DA-gGlu-OEG1-OEG2-)
PGEDAEPEELEQYYVSLRHYYNWLTRQRY-NH2

MW (calculated): 4930.5 Da

Synthesis and purification methods: S01; P01

LCMS: A01; Rt: 9.96 min; m/3: 1644.0 m/4: − m/5: −

### Compound 226

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,18Q,22V,23E,24I,28Y,30W,31L]hPYY(4-36)

(SEQ ID NO: 230)
iVal-APEK(C18DA-gGlu-OEG1-OEG2-)
PEEAASPEETQRYYVSLRHYYNWLTRQRY-NH2

MW (calculated): 4931.5 Da

Synthesis and purification methods: S01; P01

LCMS: A01; Rt: 9.26 min; m/3: 1645.0 m/4: − m/5: −

### Compound 227

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,8A,9E,18E,19Q,22V,28Y,30W,31L]hPYY(4-36)

(SEQ ID NO: 231)
iVal-APEK(C18DA-gGlu-OEG1-OEG2-)
AEEDASPEELEQYYVSLRHYYNWLTRQRY-NH2

MW (calculated): 4934.5 Da

Synthesis and purification methods: S01; P01

LCMS: A01; Rt: 10.38 min; m/3: − m/4: 1235.0 m/5: −

### Compound 228

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,18Q,19Q,22V,26A,28Y,30W,31L]hPYY(4-36)

(SEQ ID NO: 232)
iVal-APEK(C18DA-gGlu-OEG1-OEG2-)
PEEDASPEELQQYYVSLRAYYNWLTRQRY-NH2

MW (calculated): 4893.5 Da

Synthesis and purification methods: S03; P02

LCMS: A02; Rt: 12.98 min; m/3: 1632.6 m/4: 1224.3 m/5: 979.6

### Compound 229

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,18Q,22V,24A,28Y,30W,31L]hPYY(4-36)

(SEQ ID NO: 233)
iVal-APEK(C18DA-gGlu-OEG1-OEG2-)
PEEDASPEELQRYYVSARHYYNWLTRQRY-NH2

MW (calculated): 4945.5 Da

Synthesis and purification methods: S01; P01

LCMS: A01; Rt: 9.27 min; m/3: 1650.0 m/4: − m/5: −

### Compound 230

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,17I,18Q,22V,23A,28Y,30W,31L]hPYY(4-36)

iVal-APEK(C18DA-gGlu-OEG1-OEG2-)
PEEDASPEEIQRYYVALRHYYNWLTRQRY-NH2
(sequence and structure below
disclosed as SEQ ID NO: 234)

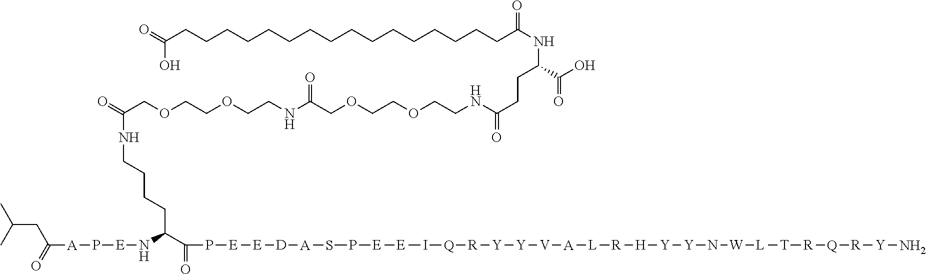

MW (calculated): 4971.6 Da

Synthesis and purification methods: S01; P01

LCMS: A01; Rt: 9.81 min; m/3: − m/4: 1243.0 m/5: −

### Compound 231

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,14A,18Q,22V,28Y,30W,31L]hPYY(4-36)

iVal-APEK(C18DA-gGlu-OEG1-OEG2-)
PEEDATPEELQKYYVSLRHYYNWLTRQRY-NH2
(sequence and structure below
disclosed as SEQ ID NO: 235)

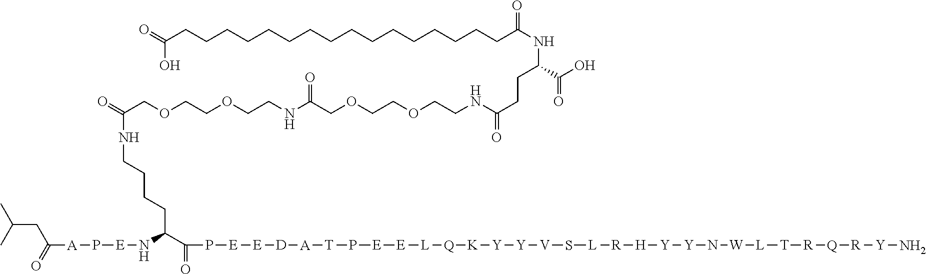

MW (calculated): 4973.6 Da

Synthesis and purification methods: S01; P01

LCMS: A01; Rt: 9.79 min; m/3: − m/4: 1245.0 m/5: −

### Compound 232

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,6A,7K,9P,18Q,22V,23E,28Y,30W,31L]hPYY(4-36)

(SEQ ID NO: 236)
iVal-APAK(C18DA-gGlu-OEG1-OEG2-)
PPEDASPEELQRYYVELRHYYNWLTRQRY-NH2

MW (calculated): 4939.6 Da

Synthesis and purification methods: S01; P01

LCMS: A01; Rt: 9.71 min; m/3: − m/4: 1235.0 m/5: −

### Compound 233

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11A,18Q,19Q,22V,28Y,30W,31L]hPYY(4-36)

(SEQ ID NO: 237)
iVal-APEK(C18DA-gGlu-OEG1-OEG2-)
PEEAASPEELQQYYVSLRHYYNWLTRQRY-NH2

MW (calculated): 4915.5 Da

Synthesis and purification methods: S03; P02

LCMS: A02; Rt: 12.58 min; m/3: 1639.6 m/4: 1230.1 m/5: 984.0

### Compound 234

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,6A,7K,9E,11E,18Q,22V,28Y,30W,31L]hPYY(4-36)

iVal-APAK(C18DA-gGlu-OEG1-OEG2-)
PEEEASPEELQRYYVSLRHYYNWLTRQRY-NH2 (sequence and
structure below disclosed as SEQ ID NO: 238)

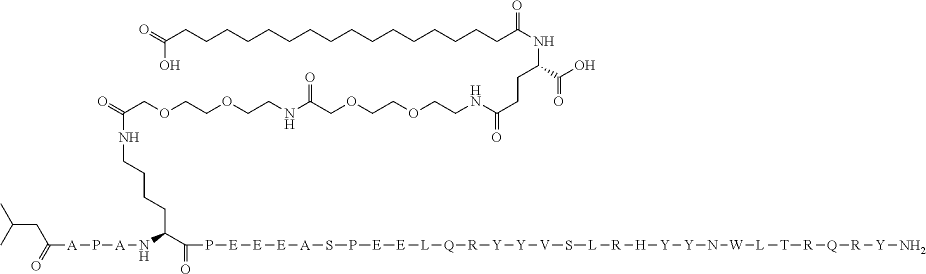

MW (calculated): 4943.6 Da

Synthesis and purification methods: S01; P01

LCMS: A01; Rt: 9.96 min; m/3: 1649.0 m/4: − m/5: −

### Compound 235

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,14Hyp,18Q,22V,28Y,30W,31L]hPYY(4-36)

(SEQ ID NO: 239)
iVal-APEK(C18DA-gGlu-OEG1-OEG2-)
PEEDASHypEELQRYYVSLRHYYNWLTRQRY-NH2

MW (calculated): 5003.6 Da

Synthesis and purification methods: S01; P01

LCMS: A01; Rt: 9.28 min; m/3: − m/4: 1252.0 m/5: −

### Compound 236

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,13P,14A,18Q,22V,28Y,30W,31L]hPYY(4-36)

(SEQ ID NO: 240)
iVal-APEK(C18DA-gGlu-OEG1-OEG2-)
PEEEAPAEELQRYYVSLRHYYNWLTRQRY-NH2

MW (calculated): 4985.6 Da

Synthesis and purification methods: S01; P01

LCMS: A01; Rt: 11.15 min; m/3: − m/4: 1247.0 m/5: −

### Compound 237

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,18Q,22V,23E,24V,28Y,30W,31L]hPYY(4-36)

(SEQ ID NO: 241)
iVal-APEK(C18DA-gGlu-OEG1-OEG2-)
PEEDASPEELQRYYVEVRHYYNWLTRQRY-NH2

MW (calculated): 5015.6 Da

Synthesis and purification methods: S01; P01

LCMS: A01; Rt: 9.60 min; m/3: 1672.0 m/4: − m/5: −

### Compound 238

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,18Q,22V,23E,24I,28Y,30W,31L]hPYY(4-36)

(SEQ ID NO: 242)
iVal-APEK(C18DA-gGlu-OEG1-OEG2-)
TEEEASPEELQRYYVSLRHYYNWLTRQRY-NH2

MW (calculated): 5005.6 Da

Synthesis and purification methods: S01; P01

LCMS: A01; Rt: 9.89 min; m/3: 1669.0 m/4: − m/5: −

### Compound 239

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,14E,18Q,21E,22V,28Y,30W,31L]hPYY(4-36)

(SEQ ID NO: 243)
iVal-APEK(C18DA-gGlu-OEG1-OEG2-)
PEEDASEEELQRYEVSLRHYYNWLTRQRY-NH2

MW (calculated): 4985.5 Da

Synthesis and purification methods: S02; P02

LCMS: A02; Rt: 11.55 min; m/3: 1662.9 m/4: 1247.5 m/5: 998.0

### Compound 240

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,16A,18Q,22V,28Y,30W,31L]hPYY(4-36)

(SEQ ID NO: 244)
iVal-APEK(C18DA-gGlu-OEG1-OEG2-)
PEEDASPEALQRYYVSLRHYYNWLTRQRY-NH2

MW (calculated): 4929.6 Da

Synthesis and purification methods: S01; P01

LCMS: A01; Rt: 9.96 min; m/3: 1643.0 m/4: − m/5: −

### Compound 241

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,17I,18Q,22V,28Y,30W,31L]hPYY(4-36)

(SEQ ID NO: 245)
iVal-APEK(C18DA-gGlu-OEG1-OEG2-)
PEEEASPEEIQRYYVSLRHYYNWLTRQRY-NH2

MW (calculated): 5001.6 Da

Synthesis and purification methods: S01; P01

LCMS: A01; Rt: 9.70 min; m/3: − m/4: 1251.0 m/5: −

### Compound 242

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,10A,14A,18Q,22V,28Y,30W,31L]hPYY(4-36)

(SEQ ID NO: 246)
iVal-APEK(C18DA-gGlu-OEG1-OEG2-)
PEADASAEELQRYYVSLRHYYNWLTRQRY-NH2

MW (calculated): 4903.5 Da

Synthesis and purification methods: S01; P01

LCMS: A01; Rt: 10.72 min; m/3: − m/4: 1226.0 m/5: −

### Compound 243

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,11E,18Q,19Q,22V,28Y,30W,31L]hPYY(4-36)

(SEQ ID NO: 247)
iVal-APEK(C18DA-gGlu-OEG1-OEG2-)
PEEEASPEELQQYYVSLRHYYNWLTRQRY-NH2

MW (calculated): 4973.6 Da

Synthesis and purification methods: S03; P02

LCMS: A02; Rt: 11.78 min; m/3: 1659.0 m/4: 1244.6 m/5: 995.5

### Compound 244

### N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]ethoxy}ethoxy) acetamido]-[4A,7K,9E,13E,15A,18Q,22V,28Y,30W,31L]hPYY(4-36)

(SEQ ID NO: 248)
iVal-APEK(C18DA-gGlu-OEG1-OEG2-)
PEEDAEPAELQRYYVSLRHYYNWLTRQRY-NH2

MW (calculated): 4971.6 Da

Synthesis and purification methods: S01; P01

LCMS: A01; Rt: 10.08 min; m/3: 1658.0 m/4: − m/5: −

The following compound disclosed as Compound 38 in WO 2016/198682 A1 was synthesized as reference:

Ref. 1
(SEQ ID NO: 249)
iVal-RPEK(C18DA-gGlu-OEG1-OEG2-)
PGEDASPEELQRYYVSLRHYYNWLTRQRY-NH2

Further compounds were synthesized as references:

Ref. 2
(SEQ ID NO: 250)
iVal-RPEK(C18DA-gGlu-OEG1-OEG2-)
PGEDASPEELQRYYVSLAHYYNWLTRQRY-NH2
Ref. 3
(SEQ ID NO: 251)
iVal-RPEK(C18DA-gGlu-OEG1-OEG2-)
PGEDASPEELQRYYVSLRAYYNWLTRQRY-NH2
Ref. 4
(SEQ ID NO: 252)
iVal-RPEK(C18DA-gGlu-OEG1-OEG2-)
PGEDASPEELQRYYVSLRHYYNWLTAQRY-NH2
Ref. 5
(SEQ ID NO: 253)
iVal-RPEK(C18DA-gGlu-OEG1-OEG2-)
PGEDASPEELQRYYVSLRHYYNWLTRQAY-NH2

The structure of Ref. 1 is—except for alanine at position 4—identical to Compound 64. This R4A mutation leads to a compound that shows much higher solubility at pH 6.2 (0.5 mg/mi for Ref. 1 vs. 8.7 mg/mi for compound 64). At the same time high binding affinity and receptor activity are maintained for compound 64 (see Table 2). Other arginine to alanine mutations at different positions (R25A in Ref. 2; R33A in Ref. 4; R35A in Ref. 5) or histidine to alanine mutation at position 26 (H26A in Ref 3) negatively affect binding affinity and receptor activity or physical stability of the resulting compounds.

TABLE 2

| | Seq. | hY2 | hY2 | Sol pH 6 | Sol pH 7 | |
|---|---|---|---|---|---|---|
| | mod. | RLB | cAMP | (mg/ml) | (mg/ml) | DLS |
| | vs. | (nM) | (nM) | [final pH] | [final pH] | pH 7.2 |
| Comp. | Ref. 1 | (Ex. 1) | (Ex. 2) | (Ex. 3) | (Ex. 3) | (Ex. 4) |
| Ref. 1 | — | 0.47 | 0.075 | 0.5 | 8.7 | n.d. |
| | | | | [6.2] | [6.9] | |
| Comp. | R4A | 0.76 | 0.157 | 8.7 | 9.6 | OK |
| 64 | | | | [6.2] | [6.8] | |
| Ref. 2 | R25A | 8.38 | 1.121 | 9.2 | 9.0 | n.d. |
| | | | | [6.0] | [6.7] | |
| Ref. 3 | H26A | 1.69 | 0.375 | 10.0 | 9.7 | Not |
| | | | | [6.0] | [6.7] | OK |
| Ref. 4 | R33A | 30.94 | 12.615 | 10.0 | 10.0 | n.d. |
| | | | | [6.1] | [6.8] | |
| Ref. 5 | R35A | 316.00 | 293.059 | 9.7 | 10.0 | n.d. |
| | | | | [6.1] | [6.8] | |

n.d.: not determined

### EXAMPLES

The following examples demonstrate certain specific embodiments of the present invention. The following examples were carried out using standard techniques that are well known and routine to those of skill in the art, except where otherwise described in detail.

### Example 1: Radioligand Binding Competition Assays (RLB)

The filtration RLB assay was carried out in 96-well plates in a final volume of 100 μl per well. Freeze-dried test peptides were dissolved in 100% dimethyl sulfoxide (DMSO) to stock solutions of 1 mM and serial dilutions were performed in assay buffer (50 mM HEPES, 5 mM MgCl₂, 1 mM CaCl₂), pH 7.4) containing 0.2% ovalbumin. 10 μl/well of the test peptide solution was added to the plates to give final concentrations ranging from 1 μM to 3 μM. Subsequently, 10 μl of human ¹²⁵I—PYY(1-36) (Perkin Elmer) in assay buffer containing 0.2% ovalbumin was added to wells to give a final concentration of 0.02 nM. Next, 80 μL membranes (HTS066M, ChemiSCREEN™ Human Neuropeptide Y2 Receptor Membrane Preparation, CHEMICON) were added to each well to give a final protein concentration of 0.5 μg/well. The plates were sealed and incubated at room temperature for 2 hours in a plate shaker set at 400 rpm. The incubation was stopped by vacuum filtration onto 0.5% polyethylene amine (PEI) presoaked GF/C filters using a 96-well FilterMate™ harvester (Perkin Elmer) followed by four washes with 300p l/well ice-cold wash buffer (50 mM HEPES, 500 mM NaCl, pH7.4). Filter plates were then dried for 60 min at room temperature and the bottom of the plates was sealed with backing tape UniFilter-96. Finally, 50 μl/well scintillation counter cocktail (Microscint20, Packard) was added and the radioactivity was counted in the Packard TopCount NXT scintillation counter. IC₅₀ values (the half maximal inhibitory concentration of the agonist) were calculated by nonlinear regression analysis of sigmoidal dose-response curves. Ki values for binding affinity were acquired by the ChengPrusoff equation (Ki=IC₅₀/(1+[L]/Kd), where Kd is the previously measured receptor specific dissociation constant (for NPY2R=0.07 nM) and [L] is ¹²⁵I—PYY(1-36) radio-ligand concentration.

The RLB results are summarized in Table 3 below.

TABLE 3

| Compound | Ki [nM] |
|---|---|
| 1 | 2.50 |
| 2 | 1.81 |
| 3 | 3.14 |
| 4 | 1.43 |
| 5 | 14.77 |
| 6 | 6.52 |
| 7 | 1.02 |
| 9 | 1.22 |
| 10 | 1.28 |
| 11 | 0.74 |
| 12 | 12.73 |
| 13 | 1.29 |
| 14 | 0.31 |
| 15 | 2.89 |
| 16 | 3.09 |
| 17 | 0.93 |
| 18 | 3.71 |
| 19 | 0.39 |
| 20 | 2.57 |
| 21 | 1.52 |
| 22 | 1.36 |
| 23 | 0.63 |
| 24 | 2.22 |
| 25 | 4.36 |
| 26 | 0.77 |
| 27 | 0.96 |
| 28 | 6.03 |
| 29 | 0.29 |
| 30 | 0.47 |
| 31 | 0.90 |
| 32 | 4.84 |
| 33 | 0.45 |
| 34 | 6.40 |
| 35 | 1.21 |
| 36 | 10.64 |
| 37 | 4.85 |
| 38 | 0.49 |
| 39 | 0.76 |
| 40 | 1.21 |
| 41 | 3.22 |
| 42 | 5.92 |
| 43 | 0.40 |
| 44 | 4.44 |
| 45 | 3.97 |
| 46 | 3.30 |
| 47 | 0.42 |
| 48 | 38.88 |
| 49 | 0.89 |
| 50 | 2.48 |
| 51 | 39.36 |
| 52 | 8.53 |
| 53 | 0.24 |
| 54 | 0.95 |
| 55 | 3.35 |
| 56 | 1.26 |
| 57 | 2.23 |
| 58 | 2.66 |
| 60 | 5.95 |
| 61 | 0.40 |
| 63 | 3.13 |
| 64 | 0.76 |
| 65 | 1.02 |
| 66 | 2.23 |
| 67 | 4.37 |
| 68 | 8.47 |
| 69 | 5.74 |
| 70 | 4.86 |
| 71 | 5.22 |
| 74 | 0.57 |
| 75 | 1.92 |
| 77 | 6.17 |
| 79 | 4.52 |
| 80 | 6.96 |
| 81 | 3.36 |
| 82 | 1.53 |
| 83 | 0.57 |
| 84 | 1.19 |
| 85 | 1.56 |
| 86 | 1.19 |
| 87 | 1.97 |
| 88 | 0.83 |
| 89 | 9.61 |
| 90 | 1.60 |
| 91 | 4.66 |
| 92 | 1.57 |
| 93 | 1.01 |
| 94 | 2.07 |
| 95 | 0.66 |
| 96 | 0.76 |
| 97 | 1.12 |
| 98 | 1.30 |
| 99 | 4.88 |
| 100 | 0.44 |
| 101 | 3.29 |
| 102 | 0.87 |
| 103 | 0.95 |
| 104 | 1.37 |
| 105 | 6.94 |
| 106 | 0.66 |
| 107 | 1.10 |
| 108 | 0.99 |
| 109 | 0.94 |
| 110 | 0.77 |
| 111 | 5.43 |
| 112 | 1.36 |
| 115 | 1.94 |
| 116 | 10.03 |
| 117 | 0.34 |
| 118 | 1.37 |
| 119 | 3.32 |
| 120 | 4.81 |
| 121 | 0.61 |
| 122 | 1.84 |
| 124 | 0.50 |
| 126 | 1.18 |
| 127 | 1.01 |
| 128 | 2.10 |
| 129 | 1.14 |
| 130 | 11.10 |
| 131 | 8.54 |
| 132 | 1.18 |
| 133 | 2.31 |
| 134 | 1.17 |
| 135 | 1.31 |
| 136 | 0.89 |
| 137 | 2.35 |
| 138 | 1.42 |
| 139 | 3.83 |
| 140 | 2.66 |
| 141 | 3.12 |
| 142 | 1.17 |
| 143 | 0.83 |
| 144 | 8.92 |
| 145 | 1.55 |
| 146 | 5.05 |
| 147 | 1.19 |
| 148 | 1.75 |
| 149 | 2.75 |
| 150 | 2.02 |
| 151 | 1.43 |
| 152 | 5.18 |
| 153 | 14.43 |
| 155 | 2.05 |
| 157 | 0.71 |
| 158 | 4.43 |
| 159 | 0.83 |
| 160 | 2.00 |
| 163 | 0.96 |
| 164 | 4.33 |
| 165 | 2.49 |
| 166 | 0.66 |
| 168 | 6.36 |
| 169 | 2.34 |
| 170 | 12.29 |
| 171 | 0.34 |
| 173 | 1.71 |
| 174 | 3.89 |
| 175 | 1.24 |
| 176 | 3.05 |
| 177 | 2.55 |
| 178 | 2.05 |
| 179 | 1.00 |
| 180 | 3.85 |
| 181 | 0.67 |
| 182 | 3.45 |
| 183 | 0.70 |
| 184 | 2.06 |
| 185 | 3.58 |
| 186 | 3.36 |
| 187 | 2.76 |
| 188 | 3.09 |
| 189 | 2.08 |
| 190 | 6.05 |
| 191 | 1.70 |
| 192 | 1.65 |
| 193 | 3.81 |
| 194 | 13.36 |
| 195 | 0.75 |
| 196 | 0.98 |
| 197 | 0.87 |
| 198 | 2.40 |
| 199 | 5.69 |
| 200 | 2.34 |
| 202 | 0.60 |
| 203 | 0.75 |
| 204 | 3.35 |
| 205 | 1.90 |
| 207 | 1.07 |
| 208 | 3.80 |
| 209 | 8.07 |
| 210 | 10.48 |
| 211 | 3.43 |
| 212 | 20.28 |
| 213 | 6.54 |
| 214 | 2.56 |
| 215 | 0.48 |
| 216 | 0.93 |
| 217 | 1.01 |
| 218 | 1.49 |
| 219 | 7.60 |
| 220 | 2.99 |
| 221 | 5.49 |
| 223 | 4.77 |
| 224 | 4.64 |
| 225 | 5.89 |
| 226 | 1.21 |
| 227 | 12.28 |
| 229 | 2.84 |
| 230 | 0.71 |
| 231 | 1.96 |
| 232 | 0.81 |
| 234 | 1.95 |
| 235 | 1.04 |
| 236 | 5.58 |
| 237 | 0.76 |
| 238 | 25.11 |
| 240 | 1.36 |
| 241 | 2.95 |
| 242 | 0.81 |
| 243 | 2.07 |
| 244 | 1.84 |

In general, the data from the RLB assay is predictive for the acute food intake inhibition in mice (Experiment 6).

### Example 2: HTRF cAMP Gi Assay

The Homogenous Time Resolved fluorescence (HTRF) technology optimized for Gi coupled receptors has thoroughly been described in Cisbio cAMP Gi kit manual. Briefly, the production of intracellular cAMP will generate a competition between unlabeled cAMP and exogenously added d2-cAMP for anti-cAMP antibodies conjugated to cryptate. CHO-KI cells stably expressing the human NPY1, NPY2, NPY4 and NPY5 receptors were used with the cells brought to life from a frozen stock immediately before assay performance. 2000 cells per well were applied for all four NPY receptor subtype assays. A 384-well format was used applying a total volume of 20 μl using 5 μl cells, 2.5 μl peptide agonist, 2.5 μl forskolin and 5 μl of each of the fluorophores. Cells were incubated with agonist peptides (11 points concentration response curves) and forskolin (~90% activity level, 3-11 μM forskolin) for 40 min at 37 °C using DPBS containing 0.5 mM IBMX as stimulation buffer. After addition of HTRF® detection reagents and incubation with shaking (2400 rpm) for one hour at room temperature signals at 620 and 665 nm (raw counts: ratio of 665/620) were detected. Concentration-response evaluation of compounds was performed with 11 concentrations of agonist peptides (covering 3 decades). EC50 values were calculated by nonlinear regression using sigmoid concentration-response with variable slope.

The in vitro activity results (expressed as EC₅₀ values) are summarized in Table 4, below.

TABLE 4

| | hY2R | hY1R | hY4R | hY5R |
|---|---|---|---|---|
| Compound NO | (nM) | (nM) | (nM) | (nM) |
| hPYY3-36 | 0.160 | 250 | >5000 | 73.2 |
| 11 | 0.334 | >5000 | >5000 | >5000 |
| 18 | 0.167 | >5000 | >5000 | >5000 |
| 23 | 0.150 | >5000 | >5000 | >5000 |
| 41 | 0.159 | >5000 | >5000 | >5000 |
| 46 | 0.152 | >5000 | >5000 | >5000 |
| 53 | 0.119 | >5000 | >5000 | >5000 |
| 54 | 0.131 | >5000 | >5000 | >5000 |
| 74 | 0.129 | >5000 | >5000 | >5000 |
| 104 | 0.096 | >5000 | >5000 | >5000 |
| 105 | 0.284 | >5000 | >5000 | >5000 |
| 106 | 0.173 | >5000 | >5000 | >5000 |
| 117 | 0.129 | >5000 | >5000 | >5000 |
| 120 | 0.210 | >5000 | >5000 | >5000 |
| 164 | 0.235 | >5000 | >5000 | >5000 |
| 171 | 0.181 | >5000 | >5000 | >5000 |
| 188 | 0.106 | >5000 | >5000 | >5000 |
| 230 | 0.199 | >5000 | >5000 | >5000 |
| 231 | 0.107 | >5000 | >5000 | >5000 |

### Example 3: Solubility Determination

Peptides (as TFA salts) were weighed out in a filter unit (Mini-UniPrep Syringeless Filter 0.45 μm, Whatman), and 0.1 M sodium diphosphate buffer at pH 6.4 or 7.0 was added to achieve 10 mg/ml final concentration. The peptide was dissolved by shaking the filter unit horizontally at 600 rpm for 2 hours at room temperature. The sample was filtered, to remove any insoluble particles and diluted to 1 mg/ml in 50% acetonitrile. The control was prepared by weighing out the corresponding peptide and dissolving it in 50% acetonitrile to final concentration of 1 mg/ml. Both the control and sample were analysed with reversed phase chromatography. The area under the peak of the sample was compared to the control and the solubility was calculated based on that ratio.

The pH was measured and recorded for each sample. Typically, buffer pH dropped by 0.2 to 0.3 pH units due to the TFA content of the peptide.

### UPLC Method:

System: UltiMate 3000 UPLC, ThermoFisher
Mobile phase A: 5% acetonitrile, 95% water, 0.03% trifluoracetic acid.
Mobile phase B: 95% acetonitrile, 5% water, 0.03% trifluoracetic acid.
Flow: 1 ml/min
Gradient: 0-100% mobile phase B (2 mins.)
Column: Kinetix, 5 μm C8, 100 Å, 50×2.1 mm
Column temperature: 50 °C

The results from the solubility determination are summarized in Table 5, below.

TABLE 5

| | Sol6 | Sol6 | Sol7 | Sol7 |
|---|---|---|---|---|
| Compound | (mg/ml) | pH | (mg/ml) | pH |
| 3 | 10.0 | 6.1 | 9.7 | 6.8 |
| 5 | 8.7 | 6.1 | 9.7 | 6.8 |
| 8 | 10.0 | 6.0 | 9.9 | 6.7 |
| 10 | 9.9 | 6.1 | 10.0 | 6.8 |
| 11 | 10.0 | 6.1 | 10.0 | 6.8 |
| 12 | 10.0 | 6.1 | 9.8 | 6.7 |
| 13 | 9.6 | 6.1 | 9.1 | 6.7 |
| 14 | 9.8 | 6.1 | 9.9 | 6.8 |
| 16 | 10.0 | 6.1 | 9.9 | 6.7 |
| 17 | 10.0 | 6.1 | 9.0 | 6.9 |
| 19 | 9.9 | 6.1 | 9.9 | 6.8 |
| 20 | 10.0 | 6.0 | 9.7 | 6.7 |
| 23 | 7.6 | 6.1 | 10.0 | 6.8 |
| 24 | 9.0 | 6.1 | 9.7 | 6.8 |
| 26 | 9.7 | 6.2 | 10.0 | 6.9 |
| 27 | 9.8 | 6.1 | 10.0 | 6.8 |
| 29 | 10.0 | 6.2 | 9.1 | 6.8 |
| 30 | 9.5 | 6.1 | 9.8 | 6.8 |
| 31 | 10.0 | 6.1 | 10.0 | 6.7 |
| 33 | 10.0 | 6.1 | 9.6 | 6.7 |
| 35 | 8.7 | 6.1 | 9.3 | 6.8 |
| 36 | 10.0 | 6.1 | 10.0 | 6.8 |
| 38 | 8.9 | 6.2 | 9.2 | 6.8 |
| 39 | 9.6 | 6.2 | 9.2 | 6.9 |
| 40 | 10.0 | 6.1 | 10.0 | 6.7 |
| 41 | 9.6 | 6.1 | 8.9 | 6.7 |
| 43 | 0.2 | 6.2 | 9.9 | 6.9 |
| 45 | 9.8 | 6.1 | 9.6 | 6.7 |
| 47 | 0.7 | 6.2 | 9.3 | 6.8 |
| 48 | 9.7 | 6.1 | 9.6 | 6.7 |
| 49 | 10.0 | 6.1 | 10.0 | 6.7 |
| 52 | 7.7 | 6.1 | 9.9 | 6.8 |
| 53 | 9.7 | 6.2 | 9.5 | 6.8 |
| 54 | 9.4 | 6.1 | 10.7 | 6.8 |
| 56 | 10.0 | 6.1 | 10.0 | 6.8 |
| 57 | 9.0 | 6.1 | 8.0 | 6.7 |
| 59 | 10.0 | 6.1 | 10.0 | 6.8 |
| 60 | 9.2 | 6.1 | 10.0 | 6.7 |
| 61 | 9.2 | 6.2 | 9.7 | 6.8 |
| 64 | 8.7 | 6.2 | 9.6 | 6.8 |
| 65 | 8.2 | 6.0 | 9.7 | 6.8 |
| 67 | 9.2 | 6.0 | 9.1 | 6.8 |
| 68 | 10.0 | 6.1 | 10.0 | 6.7 |
| 70 | 9.7 | 6.1 | 9.1 | 6.7 |
| 72 | 9.2 | 6.1 | 9.5 | 6.7 |
| 74 | 8.6 | 6.1 | 9.4 | 6.8 |
| 78 | 10.0 | 6.1 | 10.0 | 6.7 |
| 79 | 10.0 | 6.1 | 10.0 | 6.7 |
| 80 | 10.0 | 6.1 | 10.0 | 6.7 |
| 81 | 10.0 | 6.1 | 9.1 | 6.9 |
| 82 | 8.9 | 6.2 | 9.9 | 6.8 |
| 83 | 0.0 | 6.2 | 3.2 | 6.8 |
| 87 | 7.9 | 6.2 | 10.0 | 6.8 |
| 88 | 10.0 | 6.1 | 9.8 | 6.8 |
| 89 | 9.9 | 6.1 | 10.0 | 6.7 |
| 91 | 9.9 | 6.1 | 9.6 | 6.7 |
| 93 | 9.9 | 6.1 | 8.5 | 6.7 |
| 95 | 9.9 | 6.2 | 9.6 | 6.8 |
| 96 | 10.0 | 6.1 | 10.0 | 6.9 |
| 97 | 9.7 | 6.0 | 9.1 | 6.8 |
| 100 | 0.8 | 6.2 | 9.0 | 6.8 |
| 101 | 10.0 | 6.0 | 9.3 | 6.7 |
| 102 | 9.3 | 6.0 | 8.6 | 6.8 |
| 103 | 10.0 | 6.1 | 10.0 | 6.8 |
| 106 | 9.5 | 6.2 | 9.9 | 6.8 |
| 109 | 9.5 | 6.2 | 9.2 | 6.8 |
| 110 | 9.4 | 6.0 | 9.5 | 6.7 |
| 112 | 9.9 | 6.1 | 9.8 | 6.7 |
| 113 | 9.8 | 6.1 | 10.0 | 6.7 |
| 115 | 9.7 | 6.2 | 9.5 | 6.8 |
| 117 | 9.4 | 6.1 | 8.8 | 6.8 |
| 118 | 8.8 | 6.2 | 8.4 | 6.8 |
| 119 | 10.0 | 6.0 | 10.0 | 6.7 |
| 121 | 0.3 | 6.2 | 9.3 | 6.8 |
| 122 | 7.3 | 6.1 | 9.7 | 6.7 |
| 124 | 9.8 | 6.1 | 10.0 | 6.7 |
| 126 | 9.5 | 6.2 | 9.0 | 6.8 |
| 127 | 8.5 | 6.1 | 7.9 | 6.8 |
| 128 | 10.0 | 6.1 | 9.6 | 6.8 |
| 129 | 9.5 | 6.1 | 10.0 | 6.8 |
| 131 | 8.6 | 6.1 | 9.1 | 6.8 |
| 132 | 9.2 | 6.2 | 9.5 | 6.8 |
| 133 | 8.7 | 6.0 | 10.0 | 6.7 |
| 134 | 10.0 | 6.1 | 9.9 | 6.8 |
| 135 | 9.6 | 6.1 | 7.9 | 6.8 |
| 136 | 9.2 | 6.2 | 10.0 | 6.8 |
| 138 | 9.7 | 6.1 | 10.0 | 6.7 |
| 139 | 10.0 | 6.0 | 8.9 | 6.7 |
| 142 | 9.1 | 6.1 | 9.7 | 6.8 |
| 143 | 10.0 | 6.2 | 9.6 | 6.8 |
| 144 | 9.7 | 6.1 | 10.0 | 6.8 |
| 145 | 8.8 | 6.1 | 9.5 | 6.8 |
| 148 | 9.5 | 6.1 | 10.0 | 6.7 |
| 149 | 9.6 | 6.1 | 9.4 | 6.8 |
| 150 | 9.8 | 6.0 | 10.0 | 6.7 |
| 151 | 9.5 | 6.1 | 9.5 | 6.8 |
| 152 | 10.0 | 6.1 | 10.0 | 6.8 |
| 153 | 10.0 | 6.0 | 10.0 | 6.7 |
| 155 | 9.8 | 6.0 | 8.9 | 6.8 |
| 156 | 10.0 | 6.1 | 9.6 | 6.7 |
| 157 | 9.1 | 6.1 | 9.4 | 6.8 |
| 159 | 10.0 | 6.1 | 9.7 | 6.7 |
| 160 | 10.0 | 6.1 | 10.0 | 6.7 |
| 161 | 10.0 | 6.1 | 8.8 | 6.7 |
| 163 | 9.2 | 6.1 | 10.0 | 6.8 |
| 165 | 10.0 | 6.1 | 9.6 | 6.8 |
| 166 | 10.0 | 6.2 | 10.0 | 6.8 |
| 169 | 9.8 | 6.1 | 10.0 | 6.7 |
| 171 | 9.7 | 6.1 | 9.5 | 6.8 |
| 172 | 10.0 | 6.1 | 10.0 | 6.7 |
| 173 | 9.9 | 6.1 | 10.0 | 6.7 |
| 174 | 8.9 | 6.1 | 8.8 | 6.8 |
| 175 | 9.0 | 6.1 | 10.0 | 6.8 |
| 177 | 7.7 | 6.1 | 7.3 | 6.8 |
| 178 | 9.8 | 6.1 | 9.7 | 6.7 |
| 179 | 0.0 | 6.3 | 0.4 | 6.8 |
| 181 | 10.0 | 6.2 | 10.0 | 6.8 |
| 182 | 9.7 | 6.1 | 9.3 | 6.7 |
| 183 | 9.3 | 6.2 | 8.0 | 6.9 |
| 184 | 9.7 | 6.1 | 10.0 | 6.7 |
| 185 | 10.0 | 6.1 | 9.5 | 6.8 |
| 186 | 8.8 | 6.1 | 9.6 | 6.8 |
| 189 | 8.3 | 6.1 | 9.8 | 6.8 |
| 190 | 10.0 | 6.1 | 10.0 | 6.7 |
| 192 | 9.1 | 6.2 | 8.9 | 6.8 |
| 193 | 9.8 | 6.1 | 9.9 | 6.8 |
| 195 | 8.8 | 6.1 | 9.7 | 6.7 |
| 196 | 9.7 | 6.2 | 9.8 | 6.9 |
| 197 | 10.0 | 6.1 | 10.0 | 6.8 |
| 198 | 9.8 | 6.1 | 9.7 | 6.8 |
| 199 | 9.2 | 6.1 | 9.1 | 6.8 |
| 202 | 9.7 | 6.1 | 9.8 | 6.7 |
| 203 | 8.8 | 6.2 | 9.6 | 6.9 |
| 204 | 8.5 | 6.1 | 9.5 | 6.8 |
| 205 | 10.0 | 6.1 | 9.9 | 6.7 |
| 206 | 10.0 | 6.0 | 10.0 | 6.7 |
| 207 | 9.1 | 6.2 | 9.7 | 6.9 |
| 208 | 5.1 | 6.1 | 9.6 | 6.8 |
| 209 | 8.9 | 6.0 | 9.5 | 6.7 |
| 211 | 9.0 | 6.1 | 6.2 | 6.8 |
| 213 | 9.5 | 6.1 | 10.0 | 6.8 |
| 214 | 10.0 | 6.1 | 10.0 | 6.8 |
| 215 | 9.7 | 6.1 | 10.0 | 6.8 |
| 216 | 9.9 | 6.1 | 9.8 | 6.7 |
| 217 | 10.0 | 6.2 | 10.0 | 6.7 |
| 218 | 9.3 | 6.1 | 9.4 | 6.8 |
| 220 | 9.8 | 6.2 | 9.1 | 6.8 |
| 221 | 9.8 | 6.1 | 10.0 | 6.7 |
| 222 | 9.0 | 6.1 | 8.7 | 6.7 |
| 227 | 9.5 | 6.1 | 9.5 | 6.7 |
| 229 | 8.8 | 6.0 | 8.2 | 6.9 |
| 230 | 8.6 | 6.1 | 8.2 | 6.7 |
| 231 | 8.0 | 6.1 | 9.9 | 6.7 |
| 232 | 10.0 | 6.1 | 9.7 | 6.8 |
| 233 | 10.0 | 6.1 | — | — |
| 234 | 9.3 | 6.1 | 7.8 | 6.7 |
| 236 | 10.0 | 6.1 | 9.7 | 6.7 |
| 237 | 10.0 | 6.1 | 9.8 | 6.7 |
| 239 | 9.9 | 6.0 | 9.6 | 6.7 |
| 240 | 10.0 | 6.2 | 10.0 | 6.9 |
| 241 | 9.8 | 6.1 | 9.5 | 6.7 |
| 242 | 8.3 | 6.1 | 8.5 | 6.8 |
| 244 | 9.3 | 6.1 | 8.8 | 6.8 |
| Ref 1 | 0.5 | 6.2 | 8.7 | 6.9 |

### Example 4: DLS Studies

Aggregation combined with particle growth in peptide solutions was detected by dynamic light scattering (DLS). Test peptides (5 mg/ml) were dissolved in 50 mM phosphate buffer with EDTA (0.05 mg/ml) which is adjusted to a final pH of 7.2. Solutions were filtered through a 0.2 μm filter and shaken with a lab shaker at approx. 150 rpm at room temperature for 5 to 7 days. Samples were analyzed with a particle size analyzer (DLS, Horiba Nano Particle Analyzer SZ-100) at day 0 and at the end of the study. Formation of aggregates was detected by an increase in particle size over time and rated as OK, indicating no increase in particle size, and NOK, indicating an increase in particle size.

The results from the DLS studies are summarized in Table 6.

TABLE 6

| Example | DLS |
|---|---|
| 23 | OK |
| 53 | OK |
| 64 | OK |
| 106 | OK |
| 117 | OK |
| 171 | OK |
| 230 | OK |

### Example 5: Mouse PK

Pharmacokinetic parameters of the test compounds were determined after intravenous administration to NMRI mice.

Male NMRI mice were obtained either from Charles River (Germany) or from Janvier (France) weighing approximately 30 to 40 g. Mice were housed in standard cages with light cycle of 12-hour dark and 12-hour light. Standardized food and water were offered ad libitum to the animals during the whole experimental period.

The respective peptide was dissolved in 50 mM phosphate buffer (pH 7.0) containing 5% mannitol. Intravenous doses of 30 nmol/kg were given via a tail vein.

Serial blood samples were collected from the vena sephena into tubes containing EDTA as anticoagulant at different time points up to 48 h post dosing. After centrifugation for approximately 5 minutes, plasma samples were transferred to 96-well PCR plates, immediately frozen and kept at approximately −20 °C until analyzed for plasma concentration by liquid chromatography mass spectrometry (LC-MS/MS). Individual plasma concentration-time profiles were analyzed by a non-compartmental approach, and the resulting pharmacokinetic parameters were determined.

| | | Terminal |
|---|---|---|
| Compound | Mouse MRT (h) | half-life (h) |
| Ref. 1 | 11 | 8.2 |

Mouse MRT of the PYY analogues according to the invention that have been measured were comparable to Ref. 1 and show very long half-lives as compared to the half-life of hPYY(3-36).

### Example 6: Effect on Acute Food Intake in Normal NMRI Mice

Male NMRI mice were obtained from Charles River (Charles River, Research Models & Services Germany GmbH) or from JanVier (JanVier Labs, France) at 5 weeks of age. The animals were group housed 4 mice pr. cage under a 12/12 h dark-light cycle, light off at 3 PM. Room temperature was controlled to 21 °C±1 °C, with 60%±20% humidity. Animals had ad libitum access to regular rodent chow (KLIBA Nafag 3430 or Altromin 1324, Brogaarden, Denmark) and tap water.

Animals were transferred 5-7 days before the start of the study to a real-time food intake monitoring system, HM-2 system (MBRose, Denmark), to allow acclimatization to experimental conditions. As the animals were uniquely identified with microchips, each individual animal was identified by its microchip upon entry and exit from the food channel. Randomization of the mice for each study group (n=7-8) was based on body weight measured the day before the start of the study. A vehicle-treated (50 mM phosphate buffer pH7 with 5% Mannitol) group was included in each experiment. Six hours before the start of the night phase animals were fasted. One hour before the dark phase animals is were dosed once subcutaneously (10 nmol/kg) with test peptide. Food intake was reported hourly for a period of 24 h. The food intake of the treated groups was normalized (in %) to the average food intake of the group receiving vehicle (Table 7). Statistical significance was evaluated using One-way analysis of variance with Turkey's multiple comparison test. P<0.05 was considered statistically significant.

TABLE 7

| | Acute Food |
|---|---|
| | Intake 24 h |
| Compound | [% vehicle] |
| 1 | 77 |
| 3 | 62 |
| 7 | 47 |
| 9 | 44 |
| 11 | 42 |
| 14 | 46 |
| 16 | 67 |
| 18 | 58 |
| 21 | 89 |
| 23 | 48 |
| 29 | 63 |
| 30 | 45 |
| 31 | 53 |
| 33 | 54 |
| 41 | 44 |
| 46 | 39 |
| 49 | 60 |
| 53 | 29 |
| 54 | 42 |
| 56 | 53 |
| 58 | 44 |
| 74 | 41 |
| 79 | 75 |
| 90 | 70 |
| 97 | 52 |
| 101 | 63 |
| 103 | 67 |
| 104 | 61 |
| 105 | 72 |
| 106 | 39 |
| 108 | 47 |
| 110 | 67 |
| 115 | 49 |
| 117 | 45 |
| 120 | 74 |
| 124 | 58 |
| 132 | 64 |
| 133 | 57 |
| 135 | 44 |
| 139 | 74 |
| 148 | 60 |
| 150 | 67 |
| 151 | 45 |
| 154 | 73 |
| 159 | 53 |
| 163 | 50 |
| 164 | 64 |
| 167 | 73 |
| 171 | 44 |
| 173 | 49 |
| 174 | 41 |
| 176 | 52 |
| 180 | 74 |
| 185 | 39 |
| 188 | 55 |
| 193 | 57 |
| 195 | 50 |
| 202 | 48 |
| 205 | 65 |
| 208 | 61 |
| 209 | 50 |
| 211 | 62 |
| 221 | 61 |
| 230 | 44 |
| 231 | 39 |
| 234 | 42 |
| 241 | 45 |
| 244 | 49 |
| Ref. 1 | 41 |

### Example 7: Activity after Incubation in Solutions at Different pH Values

Peptides (as TFA salts) were weighed out in a filter unit (Mini-UniPrep Syringeless Filter 0.45 μm, Whatman), and 0.1 M sodium diphosphate buffer at pH 6.4 or 7.4, respectively, or 0.2 M TRIS buffer at pH 8.3 was added to achieve 10 mg/ml final concentration. The peptide was (partially) dissolved by shaking the filter unit horizontally at approx. 400 rpm for 2 hours at room temperature. The sample was filtered to remove any insoluble particles. The filtrate was subsequently subjected to the binding assay as described in Example 1. The apparent binding affinity (Ki) reported in Table 8 is calculated based on an assumed concentration of 10 mg/ml.

Compounds of the invention are active in the binding assay after incubation in solutions at different pH values, including pH 6. This demonstrates the fundamental feasibility of a liquid formulation of the compounds according to the invention at ~pH 6 (in the pH range from ~6 to ~8).

Contrary hereto, after incubation at ~pH 6, solutions of Ref. 1 (after filtration) show a weak response in the binding assay (apparent significant (>35×) affinity loss due to low solubility of Ref. 1 in buffered media at pH 6). Therefore, the low intrinsic solubility of Ref. 1 at ~pH 6 limits the formulation space in the physiological pH range of 6-8 of a liquid formulation of Ref. 1.

TABLE 8

| | hY2R | RLB 6 | RLB 7 | RLB 8 | RLB | RLB |
|---|---|---|---|---|---|---|
| | RLB | (pH6.1- | (pH7.1- | (pH8.1- | ratio | ratio |
| | Ki (nM) | 6.2) | 7.2) | 8.2) | (RLB6/ | (RLB8/ |
| Cpd. | DMSO | (nM) | (nM) | (nM) | RLB7) | RLB7) |
| Ref. 1 | 3.0 | >216 | 2.9 | 2.4 | >74 | 0.8 |
| 23 | 4.9 | 6.3 | 6.3 | 6.3 | 1.0 | 1.0 |
| 29 | 4.6 | 3.8 | 4.4 | 2.3 | 0.9 | 0.5 |
| 53 | 2.0 | 3.4 | 3.1 | 2.5 | 1.1 | 0.8 |
| 84 | 3.5 | 6.6 | 2.9 | 4.1 | 2.3 | 1.4 |
| 106 | 6.0 | 7.7 | 9.3 | 7.5 | 0.8 | 0.8 |
| 117 | 4.1 | 6.7 | 2.1 | 8.8 | 3.2 | 4.2 |
| 171 | 6.7 | 3.0 | 6.1 | 5.6 | 0.5 | 0.9 |
| 203 | 3.0 | 4.6 | 11.1 | 8.8 | 0.4 | 0.8 |

What is claimed is:

1. A PYY analogue comprising an amino acid sequence corresponding to the amino acid sequence of hPYY(3-36), wherein the analogue comprises
i) alanine at the position corresponding to position 4 of hPYY(1-36)(SEQ ID NO: 2) ii) lysine at the position corresponding to position 7 of hPYY(1-36) (SEQ ID NO: 2) iii) glutamate at the position corresponding to position 9 of hPYY(1-36) (SEQ ID NO: 2)
iv the sequence QRY at its C-terminal end, and wherein a half-life extending group is attached to the epsilon amino group of the lysine corresponding to position 7 of hPYY(1-36) (SEQ ID NO: 2) or of a lysine corresponding to position 6, 10, 11, 14, 17, 21, 22 or 30 of hPYY(1-36) (SEQ ID NO: 2), or to the carboxylic acid group of the side chain of a aspartate or a glutamate corresponding to position 14 or 30 of hPYY(1-36) (SEQ ID NO: 2), wherein the half-life extending group consists of a lipophilic substituent X and a linker U, wherein the linker U is attached to the epsilon amino group of lysine or to the carboxylic acid group of the side chain of aspartate or glutamate and X is attached to U;
the linker U comprises one or more sub-moieties selected from the group consisting of Ala, Glu, γ-Glu, Gly, ε-Lys, Ser, OEG and OEG-OEG, or a pharmaceutically acceptable salt thereof.

2. The PYY analogue according to claim 1, wherein the analogue comprises
tyrosine at the position corresponding to position 20 of hPYY(1-36)(SEQ ID NO: 2) and/or
arginine at the position corresponding to position 25 of hPYY(1-36)(SEQ ID NO: 2) and/or
tryptophane at the position corresponding to position 30 of hPYY(1-36)(SEQ ID NO: 2), and/or
leucine at the position corresponding to position 31 of hPYY(1-36)(SEQ ID NO: 2), and/or
threonine at the position corresponding to position 32 of hPYY(1-36)(SEQ ID NO: 2), and/or
arginine or lysine at the position corresponding to position 33 of hPYY(1-36)(SEQ ID NO: 2), or a pharmaceutically acceptable salt thereof.

3. A PYY analogue having the formula:
R¹—Z—R², wherein R¹ is hydrogen, —C(O)C₁₋₆ alkyl, —C(O)C₆H₆, —C(O)C₃₋₆ cycloalkyl, —C(O)C₁₋₆ alkyl-C₃₋₆ cycloalkyl, or C₁₋₆ alkyl or C₁₋₆ alkyl-C₃₋₆ cycloalkyl: R² is OH or NHR³, wherein R³ is hydrogen or C₁₋₃ alkyl; and Z is a peptide comprising an amino acid sequence of formula I (SEQ ID NO: 254): Ala-X5-X6-Lys-X8-X9-X10-X11-X12-X13-X14-X15-X16-X17-X18-X19-Tyr-X21-X22-X23-X24-Arg-X26-X27-X28-X29-X30-X31-X32-Arg-Gln-Arg-Tyr (I) wherein X5 is selected from the group consisting of Pro and Hyp; X6 is selected from the group consisting of Ala and Glu; X8 is selected from the group consisting of Ala, Ile, Pro, Thr, Val and Hyp; X9 is Glu; X10 is selected from the group consisting of Ala and Glu; X11 is selected from the group consisting of Ala, Asp, is Glu and Pro; X12 is selected from the group consisting of Ala, Gly, Ser, Thr and Val; X13 is selected from the group consisting of Ala, Glu, Ser, Gln, Thr and Pro; X14 is selected from the group consisting of Ala, Glu, Gly, Pro and Hyp; X15 is selected from the group consisting of Ala, Glu and Ser; X16 is selected from the group consisting of Ala, Glu and Ser; X17 is selected from the group consisting of Ala, Ile, Leu, Thr and Val; X18 is selected from the group consisting of Glu and Gln; X19 is selected from the group consisting of Ala, Glu, Arg, Lys and GlnX21 is selected from the group consisting of Ala, Glu, Gln and Tyr; X22 is selected from the group consisting of Ile, Ser, Thr and Val; X23 is selected from the group consisting of Ala, Glu, Ser and Thr; X24 is selected from the group consisting of Ala, Ile, Leu, Thr and Val; X26 is selected from the group consisting of Ala, His and Lys; X27 is selected from the group consisting of Gln and Tyr; X28 is selected from the group consisting of His, Trp and Tyr; X29 is selected from the group consisting of Asn, Trp and Tyr; X30 is selected from the group consisting of Ala, His, Trp, and Tyr; X31 is selected from the group consisting of Ala, Ile, Leu and Thr; X32 is selected from the group consisting of Gln, Leu and Thr; wherein one to three amino acids of X5, X6, X8-X19, X21-X24 and X26-X32 may be absent, and wherein a half-life extending group is attached to the epsilon amino group of the lysine at position 7, wherein the half-life extending group consists of a lipophilic substituent X and a linker U, wherein the linker U is attached to the epsilon amino group of lysine or to the carboxylic acid group of the side chain of aspartate or glutamate and X is attached to U;
the linker U comprises one or more sub-moieties selected from the group consisting of Ala, Glu, γ-Glu, Gly, ε-Lys, Ser, OEG and OEG-OEG, or a pharmaceutically acceptable salt thereof.

4. The PYY analogue according to claim 3, wherein the PYY analogue is a compound having the formula:
R¹—Z—R², wherein R¹ is hydrogen, —C(O)C₁₋₆ alkyl, —C(O)C₆H₆, —C(O)C₃₋₆ cycloalkyl, —C(O)C₁₋₆ alkyl-C₃₋₆ cycloalkyl, or C₁₋₆ alkyl or C₁₋₆ alkyl-C₃₋₆ cycloalkyl: R² is OH or NHR³, wherein R³ is hydrogen or C₁₋₃ alkyl; and Z is an amino acid sequence of formula III (SEQ ID NO: 256): Ala-Pro-X6-Lys-Pro-X9-X10-X11-X12-X13-X14-X15-X16-X17-X18-X19-Tyr-X21-Val-X23-Leu-Arg-His-Tyr-Tyr-Asn-Trp-LeuThr-Arg-Gln-Arg-Tyr (III) wherein X6 is selected from the group consisting of Ala and Glu; X9 is Glu; X10 is selected from the group consisting of Ala and Glu; X11 is selected from the group consisting of Ala, Asp, Glu and Pro; X12 is selected from the group consisting of Ala and Ser; X13 is selected from the group consisting of Ala, Glu, Ser, Thr and Pro; X14 is selected from the group consisting of Ala, Glu and Pro; X15 is selected from the group consisting of Ala and Glu, X16 is selected from the group consisting of Ala and Glu; X17 is selected from the group consisting of Ile, Leu, Thr and Val; X18 is selected from the group consisting of Glu and Gln; X19 is selected from the group consisting of Ala, Glu, Arg, Lys, and Gln; X21 is selected from the group consisting of Glu and Tyr; X23 is selected from the group consisting of Ala, Glu, Ser and Thr; and wherein a half-life extending group is attached to the epsilon amino group of the lysine at position 7, or a pharmaceutically acceptable salt thereof.

5. The PYY analogue according to claim 3, wherein the PYY analogue is a compound having the formula:
R¹—Z—R², wherein R¹ is hydrogen, —C(O)C₁₋₆ alkyl, —C(O)C₆H₆, —C(O)C₃₋₆ cycloalkyl or C₁₋₆ alkyl; R² is OH or NHR³, wherein R³ is hydrogen or C₁₋₃ alkyl; and Z is an amino acid sequence selected from the group consisting of SEQ ID Nos. 5 to 29, 32 to 41, 44 to 50, 52 to 64, 67, 69 to 79, 81 to 91, 93 to 103, 105 to 123, 125 to 128, 130 to 143, 145, 147 to 169, 171 to 186, 188 to 199, 201 to 204, 206 to 227, 230 to 235, and 237 to 248,
or a pharmaceutically acceptable salt thereof.

6. The PYY analogue according to claim 3, wherein at least two amino acids from X6, X10, X11, X13 and X23 are selected from the group consisting of Asp and Glu,
or a pharmaceutically acceptable salt thereof.

7. The PYY analogue according to claim 3, wherein only one of X6, X10 and X15 is Ala,
or a pharmaceutically acceptable salt thereof.

8. The PYY analogue according to claim 3, wherein X is selected from the group consisting of 15-carboxy-pentadecanoyl, 17-carboxy-heptadecanoyl (C18DA) and 19-carboxy-nonadecanoyl, and U consists of one, two or three sub-moieties independently selected from the group consisting of Gly, Glu, γ-Glu, ε-Lys, Ser and OEG, or a pharmaceutically acceptable salt thereof.

9. The PYY analogue according to claim 3 or a pharmaceutically acceptable salt thereof, wherein the PYY analogue is a compound having an amino acid sequence selected from the group consisting of
TABLE 1

| | | SEQ ID |
|---|---|---|
| Compound | Sequence | NO |
| 1 | APEKPEEDAQPEELQEYYVSLRHYYNWLTRQRY | 5 |
| 2 | AHypEKPEEDASPEELQRYYVSLRHYYNWLTRQRY | 6 |
| 3 | APEKPEEEASPAELQRYYVSLRHYYNWLTRQRY | 7 |
| 4 | APEKPEEDASPEELQRYYVSLRHYYNWLQRQRY | 8 |
| 5 | APEKPEEEVSPEELQRYYVSLRHYYNWLTRQRY | 9 |
| 6 | APEKPEEEASPEELQRYYVSARHYYNWLTRQRY | 10 |
| 7 | APEKPEEDASPEELQAYYVSLRHYYNWLTRQRY | 11 |
| 8 | APEKPEEDASEEELQQYYVSLRHYYNWLTRQRY | 12 |
| 9 | APEKPEEDASPEEIQRYYVELRHYYNWLTRQRY | 13 |
| 10 | APEKPEADASPEETQRYYVSLRHYYNWLTRQRY | 14 |
| 11 | APEKPEEDASPEEIQQYYVSLRHYYNWLTRQRY | 15 |
| 12 | APEKPEEDAPGEELQRYYVSLRHYYNWLTRQRY | 16 |
| 13 | APEKPEEDAAPEELQQYYVSLRHYYNWLTRQRY | 17 |
| 14 | APEKPEADATPEELQRYYVSLRHYYNWLTRQRY | 18 |
| 15 | APEKPEEDASPEEIQRYEVSLRHYYNWLTRQRY | 19 |
| 16 | APEKPEEDASPEETQRYYVTLRHYYNWLTRQRY | 20 |
| 17 | APEKPEEDASPAELQRYYVSLRHYYNWLTRQRY | 21 |
| 18 | APEKPEEDAEPEELQQYYVSLRHYYNWLTRQRY | 22 |
| 19 | APEKPEADASPEELQQYYVSLRHYYNWLTRQRY | 23 |
| 20 | APEKPEEDAAPEELQRYYVELRHYYNWLTRQRY | 24 |
| 21 | APEKPEEDASPEELQKYYVSLRHYYNWLTRQRY | 25 |
| 22 | APEKPEEDASPEELQRYYTSLRHYYNWLTRQRY | 26 |
| 23 | APAKPEEDASPEEIQRYYVSLRHYYNWLTRQRY | 27 |
| 24 | APEKPEEDASPEELQRYYVSLRKYYNWLTRQRY | 28 |
| 25 | APEKPEEEASAEELQRYYVSLRHYYNWLTRQRY | 29 |
| 26 | APEKPPEDATPEELQRYYVSLRHYYNWLTRQRY | 30 |
| 27 | APEKPPEDAEPEELQRYYVSLRHYYNWLTRQRY | 31 |
| 28 | APEKPEEDTSPEELQRYYVELRHYYNWLTRQRY | 32 |
| 29 | APEKPEAPASPEELQRYYVELRHYYNWLTRQRY | 33 |
| 30 | APEKPEADASEEELQRYYVSLRHYYNWLTRQRY | 34 |
| 31 | APEKPEEDASPEETQRYYVALRHYYNWLTRQRY | 35 |
| 32 | APEKPEEDTSPEELQRYEVSLRHYYNWLTRQRY | 36 |
| 33 | APEKPEEPASPEELQRYYVELRHYYNWLTRQRY | 37 |
| 34 | APEKPEEDASPEELQRYYVSTRHYYNWLTRQRY | 38 |
| 35 | APEKPEEDAAPEETQRYYVSLRHYYNWLTRQRY | 39 |
| 36 | APEKAEEDAEPEELQRYYVSLRHYYNWLTRQRY | 40 |
| 37 | APEKPEEDASPEETQRYEVSLRHYYNWLTRQRY | 41 |
| 38 | APEKPPEDASPEELQRYYVALRHYYNWLTRQRY | 42 |
| 39 | APEKPPADAEPEELQRYYVSLRHYYNWLTRQRY | 43 |
| 40 | APEKPEEDATPEETQRYYVSLRHYYNWLTRQRY | 44 |
| 41 | APEKPEEEATPEELQRYYVSLRHYYNWLTRQRY | 45 |
| 42 | APEKPEEAASPEELQRYYVSTRHYYNWLTRQRY | 46 |
| 43 | APEKPEAPASPEELQRYYVSLRHYYNWLTRQRY | 47 |
| 44 | APEKPEEEASPEELQRYYVSLRHYYNWITRQRY | 48 |
| 45 | APEKPEEEASPESLQRYYVSLRHYYNWLTRQRY | 49 |
| 46 | APEKPEEEASPEELQRYYVSLRHYYNWLTRQRY | 50 |
| 47 | APEKPPADASPEELQRYYVSLRHYYNWLTRQRY | 51 |
| 48 | APEKPEEDTEPEELQRYYVSLRHYYNWLTRQRY | 52 |
| 49 | APEKPEEDASPEELQQYYVELRHYYNWLTRQRY | 53 |
| 50 | APEKPEEDASAEELQRYYVSLRHYYNWITRQRY | 54 |
| 51 | APEKAEEEASPEELQRYYVSLRHYYNWLTRQRY | 55 |
| 52 | APEKAEEDASEEELQRYYVSLRHYYNWLTRQRY | 56 |
| 53 | APEKPEADASPEEIQRYYVSLRHYYNWLTRQRY | 57 |
| 54 | APEKPEEDASPEELQRYYVSLRHYYNWLTRQRY | 58 |
| 55 | APEKPEEDASPEEAQRYYVSLRHYYNWLTRQRY | 59 |
| 56 | APEKPEADAEPEELQRYYVSLRHYYNWLTRQRY | 60 |
| 57 | APEKPEEDASAEEIQRYYVSLRHYYNWLTRQRY | 61 |
| 58 | APEKPEEDASPEELQQYYVSLRHYYNWLTRQRY | 62 |
| 59 | APEKPEEDASPEETEQYYVSLRHYYNWLTRQRY | 63 |
| 60 | APEKPEEAASPEELEQYYVSLRHYYNWLTRQRY | 64 |
| 61 | APEKPPEDASPEEIQRYYVSLRHYYNWLTRQRY | 65 |
| 62 | APEKPGEDASPEELERYYVSLRHYYNWLTRQRY | 66 |
| 63 | APEKPEEDASPEELQRYYVSLRAYYNWLTRQRY | 67 |
| 64 | APEKPGEDASPEELQRYYVSLRHYYNWLTRQRY | 68 |
| 65 | APEKPEEDASPEELQRYYVEIRHYYNWLTRQRY | 69 |
| 66 | APEKPEEDASAEETQRYYVSLRHYYNWLTRQRY | 70 |
| 67 | APEKPEEDSAPEELQRYYVSLRHYYNWLTRQRY | 71 |
| 68 | APEKPEEEASPEELQAYYVSLRHYYNWLTRQRY | 72 |
| 69 | APEKPEEDSSAEELQRYYVSLRHYYNWLTRQRY | 73 |
| 70 | APEKPEEAASPEELQRYEVSLRHYYNWLTRQRY | 74 |
| 71 | APEKPEEEASPSELQRYYVSLRHYYNWLTRQRY | 75 |
| 72 | APEKPEEEGSPEELQRYYVSLRHYYNWLTRQRY | 76 |
| 73 | APEKPEEEASPEELQRYYVSLRAYYNWLTRQRY | 77 |
| 74 | APEKPEEDASAEELQRYYVALRHYYNWLTRQRY | 78 |
| 75 | APEKPEEDASAEELQRYYVTLRHYYNWLTRQRY | 79 |
| 76 | APEKPGEDASPEELQRYYVSLRHYYNALTRQRY | 80 |
| 77 | APEKPEEDASEEELQRYYVELRHYYNWLTRQRY | 81 |
| 78 | APEKPEEDAEPEELQQYYVTLRHYYNWLTRQRY | 82 |
| 79 | APEKPEEEASEEELQRYYVSLRHYYNWLTRQRY | 83 |
| 80 | APEKPEEEASPEETQRYYVSLRHYYNWLTRQRY | 84 |
| 81 | APEKPEEDASPEELQRYYVSIRHYYNWLTRQRY | 85 |
| 82 | APEKPEEDASPEALQRYYVALRHYYNWLTRQRY | 86 |
| 83 | APEKPEADASPAELQRYYVSLRHYYNWLTRQRY | 87 |
| 84 | APEKPEEDASPEEIQRYYVSLRHYYNWLTRQRY | 88 |
| 85 | APEKPEEDASPEELQRYAVSLRHYYNWLTRQRY | 89 |
| 86 | APEKPEADASPEELQRYEVSLRHYYNWLTRQRY | 90 |
| 87 | APEKPEEDSSPAELQRYYVSLRHYYNWLTRQRY | 91 |
| 88 | APEKPPEDASPEELQRYYVELRHYYNWLTRQRY | 92 |
| 89 | APEKPEEEAEPEELQRYYVSLRHYYNWLTRQRY | 93 |
| 90 | APEKPEEDASPEELQRYEVSLRHYYNWLTRQRY | 94 |
| 91 | APEKPEEDAPAEELQRYYVELRHYYNWLTRQRY | 95 |
| 92 | APEKPEEDTSPEELQRYYVSLRHYYNWLTRQRY | 96 |
| 93 | APEKPEEDASAEELQKYYVSLRHYYNWLTRQRY | 97 |
| 94 | APEKPEEDASPEETQRYYVELRHYYNWLTRQRY | 98 |
| 95 | APEKPEEDASPAEIQRYYVSLRHYYNWLTRQRY | 99 |
| 96 | APEKPEADASPEELQRYYVSLRHYYNWLTRQRY | 100 |
| 97 | APEKPEEDASPEELQRYYVALRHYYNWLTRQRY | 101 |
| 98 | APEKPEEDASPEELQRYYSALRHYYNWLTRQRY | 102 |
| 99 | APEKPEEDAEAEELQRYYVSLRHYYNWLTRQRY | 103 |
| 100 | APAKPPEDASPEELQRYYVSLRHYYNWLTRQRY | 104 |
| 101 | APEKPEEDATPEELQRYYVELRHYYNWLTRQRY | 105 |
| 102 | APEKPEEDASPEELQRYYVTLRHYYNWLTRQRY | 106 |
| 103 | APAKPEEDASEEELQRYYVSLRHYYNWLTRQRY | 107 |
| 104 | APEKPEEDASAEELQRYYVSLRHYYNWLTRQRY | 108 |
| 105 | APEKPEEDAEPEELQEYYVSLRHYYNWLTRQRY | 109 |
| 106 | APAKPEEDATPEELQRYYVSLRHYYNWLTRQRY | 110 |
| 107 | APEKPEEDASPEELQKYYVALRHYYNWLTRQRY | 111 |
| 108 | APEKPEEDASPEELQRYYVELRHYYNWLTRQRY | 112 |
| 109 | APEKPEEAASPEELQRYYVSLRHYYNWLTRQRY | 113 |
| 110 | APEKPEEDAEPEEIQRYYVSLRHYYNWLTRQRY | 114 |
| 111 | APEKPEEDAEPEELQRYYVELRHYYNWLTRQRY | 115 |
| 112 | APEKPEEDATPEELQRYYVSLRHYYNWLTRQRY | 116 |
| 113 | APEKAEEDATPEELQRYYVSLRHYYNWLTRQRY | 117 |
| 114 | APEKPEEDASPEELQRYYVSLRHYHWYLTRQRY | 118 |
| 115 | APEKPEEEASPEALQRYYVSLRHYYNWLTRQRY | 119 |
| 116 | APEKPEEDAAAEELQRYYVSLRHYYNWLTRQRY | 120 |
| 117 | APAKPEEDASPEELQRYYVALRHYYNWLTRQRY | 121 |
| 118 | APEKPEEDASAAELQRYYVSLRHYYNWLTRQRY | 122 |
| 119 | APEKPEEEASPEELQRYYVTLRHYYNWLTRQRY | 123 |
| 120 | APEKPGEDAEEEELQEYYVSLRHYYNWLTRQRY | 124 |
| 121 | APAKPEADASPEELQRYYVSLRHYYNWLTRQRY | 125 |
| 122 | APEKPEEDAAPEELQRYYVSLRHYYNWLTRQRY | 126 |
| 123 | APEKPEEDASPEELQRYYVSLRHYWYHLTRQRY | 127 |
| 124 | APEKPEEDAAPEEIQRYYVSLRHYYNWLTRQRY | 128 |
| 125 | APEKPGEDASPEELQRYYVSLRHYYNWATRQRY | 129 |
| 126 | APAKPEEDSSPEELQRYYVSLRHYYNWLTRQRY | 130 |
| 127 | APAKPEEDASAEELQRYYVSLRHYYNWLTRQRY | 131 |
| 128 | APEKPEEDAPAEEIQRYYVSLRHYYNWLTRQRY | 132 |
| 129 | APEKPEEDASPEETQRYYTALRHYYNWLTRQRY | 133 |
| 130 | APEKPEEDTSAEELQRYYVSLRHYYNWLTRQRY | 134 |
| 131 | APEKAEEDASPEELQRYYVELRHYYNWLTRQRY | 135 |
| 132 | APEKPEEDASPEELQKYYVTLRHYYNWLTRQRY | 136 |
| 133 | APEKPEEDASPEEVQRYYVELRHYYNWLTRQRY | 137 |
| 134 | APEKPEEDASPEEIQRYYTELRHYYNWLTRQRY | 138 |
| 135 | APEKPEADASPEELQRYYVELRHYYNWLTRQRY | 139 |
| 136 | APEKPEADSSPEELQRYYVSLRHYYNWLTRQRY | 140 |
| 137 | APEKPEEDASPEELQRYYVSLRHYYNWITRQRY | 141 |
| 138 | APAKPEEDASPEELQQYYVSLRHYYNWLTRQRY | 142 |
| 139 | APEKPEEESSPEELQRYYVSLRHYYNWLTRQRY | 143 |
| 140 | APEKPGEDASPEELEQYYVSLRHYYNWLTRQRY | 144 |
| 141 | APEKPEEAASPEELQRYYVSARHYYNWLTRQRY | 145 |
| 142 | APEKPPEEASPEELQRYYVSLRHYYNWLTRQRY | 146 |
| 143 | APEKPEEDATPEELQRYYVALRHYYNWLTRQRY | 147 |
| 144 | APEKAEEDASPEELQQYYVSLRHYYNWLTRQRY | 148 |
| 145 | APEKPEEDSSPEEIQRYYVSLRHYYNWLTRQRY | 149 |
| 146 | APEKPEEEASPEELQRYYVSTRHYYNWLTRQRY | 150 |
| 147 | APEKPEEDASPEETQKYYVSLRHYYNWLTRQRY | 151 |
| 148 | APEKPEEDAPAEELQRYYVSLRHYYNWLTRQRY | 152 |
| 149 | APEKPEEAASPEELQRYYVELRHYYNWLTRQRY | 153 |
| 150 | APEKPEEDAEPEELQKYYVSLRHYYNWLTRQRY | 154 |
| 151 | APEKPEAEASPEELQRYYVSLRHYYNWLTRQRY | 155 |
| 152 | APEKAEEDASPEEIQRYYVSLRHYYNWLTRQRY | 156 |
| 153 | APEKVEEEASPEELQRYYVSLRHYYNWLTRQRY | 157 |
| 154 | APEKPEEEASPEELQRYEVSLRHYYNWLTRQRY | 158 |
| 155 | APEKPEEDASPEETQRYYVSLRHYYNWLTRQRY | 159 |
| 156 | APEKPEEDASEEELEQYYVSLRHYYNWLTRQRY | 160 |
| 157 | APAKPEEDASPEETQRYYVSLRHYYNWLTRQRY | 161 |
| 158 | APEKPEEDASPEELQRYYVSLRHYWNYLTRQRY | 162 |
| 159 | APEKPEADASPEELEQYYVSLRHYYNWLTRQRY | 163 |
| 160 | APEKPEADAEPEELQRYYTSLRHYYNWLTRQRY | 164 |
| 161 | APEKPEEDATPEELQQYYVSLRHYYNWLTRQRY | 165 |
| 162 | APEKPEEDASPEELQRYEVSLRAYYNWLTRQRY | 166 |
| 163 | APEKPEEAASPEELQRYYVALRHYYNWLTRQRY | 167 |
| 164 | APEKPEEDASEEELQEYYVSLRHYYNWLTRQRY | 168 |
| 165 | APEKPEEDASPEELQRYQVSLRHYYNWLTRQRY | 169 |
| 166 | APEKPPADASPEELQRYYVELRHYYNWLTRQRY | 170 |
| 167 | APEKPEEDASPEELQQYEVSLRHYYNWLTRQRY | 171 |
| 168 | APEKPEEDAEPEELQRYYVSLRHYYNWITRQRY | 172 |
| 169 | APEKPEEEASPEELQRYYVALRHYYNWLTRQRY | 173 |
| 170 | APEKPEEEASPEELEQYYVSLRHYYNWLTRQRY | 174 |
| 171 | APEKPEEDATPEEIQRYYVSLRHYYNWLTRQRY | 175 |
| 172 | APEKPEEDAEPEELQRYYVALRHYYNWLTRQRY | 176 |
| 173 | APAKPEEDASPEELQRYYVELRHYYNWLTRQRY | 177 |
| 174 | APAKPEEDAEPEELQRYYVSLRHYYNWLTRQRY | 178 |
| 175 | APEKPEEDAAPEELQRYYVALRHYYNWLTRQRY | 179 |
| 176 | APEKPEEDASEEELQRYYVSLRHYYNWLTRQRY | 180 |
| 177 | APEKPEEDSSPEELQRYYVELRHYYNWLTRQRY | 181 |
| 178 | APEKPEEDATPEETERYYVSLRHYYNWLTRQRY | 182 |
| 179 | APAKPEEDASPAELQRYYVSLRHYYNWLTRQRY | 183 |
| 180 | APEKPEEDASPEETQQYYVSLRHYYNWLTRQRY | 184 |
| 181 | APEKPEADASPEELQRYYVALRHYYNWLTRQRY | 185 |
| 182 | APEKPEEDGSPEELQRYYVELRHYYNWLTRQRY | 186 |
| 183 | APEKPPEDASPEELQRYYVSLRHYYNWLTRQRY | 187 |
| 184 | APEKPEEAASPEELQRYYVTLRHYYNWLTRQRY | 188 |
| 185 | APEKPEEEASPEELQRYYVELRHYYNWLTRQRY | 189 |
| 186 | APEKPEEDASAEELQRYYVELRHYYNWLTRQRY | 190 |
| 187 | APEKPEEDASPEELQRYYVSLRHQYNWLTRQRY | 191 |
| 188 | APEKPEEDAEPEELQRYYVSLRHYYNWLTRQRY | 192 |
| 189 | APEKPEEDASEAELQRYYVSLRHYYNWLTRQRY | 193 |
| 190 | APEKPEEEAPPEELQRYYVSLRHYYNWLTRQRY | 194 |
| 191 | APEKPEEDASPEELQRYYISLRHYYNWLTRQRY | 195 |
| 192 | APEKPEEDAAPAELQRYYVSLRHYYNWLTRQRY | 196 |
| 193 | APEKPEEDASEEEIQRYYVSLRHYYNWLTRQRY | 197 |
| 194 | AHypEKPEEDASPEELQRYYVSLRHYYNWTLRQRY | 198 |
| 195 | APEKPEEDASPEALQRYYVELRHYYNWLTRQRY | 199 |
| 196 | APAKPPEDAEPEELQRYYVSLRHYYNWLTRQRY | 200 |
| 197 | APAKPEADASPEELQRYYVELRHYYNWLTRQRY | 201 |
| 198 | APEKPEEDAEPEALQRYYVSLRHYYNWLTRQRY | 202 |
| 199 | APEKPEEDAEPEETQRYYVSLRHYYNWLTRQRY | 203 |
| 200 | APEKHypEEDASPEELQRYYVSLRHYYNWLTRQRY | 204 |
| 201 | APEKPQEDAEPEELQEYYVSLRHYYNWLTRQRY | 205 |
| 202 | APEKPEEDAAPEELQKYYVSLRHYYNWLTRQRY | 206 |
| 203 | APAKPEEDASPEELQRYYVSLRHYYNWLTRQRY | 207 |
| 204 | APEKPEEEASPEEVQRYYVSLRHYYNWLTRQRY | 208 |
| 205 | APEKPEEEASPEELQKYYVSLRHYYNWLTRQRY | 209 |
| 206 | APEKPEEDASPEELEQYEVSLRHYYNWLTRQRY | 210 |
| 207 | APAKPEADAEPEELQRYYVSLRHYYNWLTRQRY | 211 |
| 208 | APEKPEEDASEEETQRYYVSLRHYYNWLTRQRY | 212 |
| 209 | APEKPEEDSEPEELQRYYVSLRHYYNWLTRQRY | 213 |
| 210 | APEKIEEEASPEELQRYYVSLRHYYNWLTRQRY | 214 |
| 211 | APEKPEEDATAEELQRYYVSLRHYYNWLTRQRY | 215 |
| 212 | APEKPEEDTAPEELQRYYVSLRHYYNWLTRQRY | 216 |
| 213 | APEKPEEETSPEELQRYYVSLRHYYNWLTRQRY | 217 |
| 214 | APEKPEEDASEEELQRYYVALRHYYNWLTRQRY | 218 |
| 215 | APEKPEEDASPAELQRYYVELRHYYNWLTRQRY | 219 |
| 216 | APAKPEEDASPEELEQYYVSLRHYYNWLTRQRY | 220 |
| 217 | APEKPEEDASPAELQRYYVALRHYYNWLTRQRY | 221 |
| 218 | APEKPEEDSSPEELQRYYVALRHYYNWLTRQRY | 222 |
| 219 | APEKPEEEAAPEELQRYYVSLRHYYNWLTRQRY | 223 |
| 220 | APEKPEEAASAEELQRYYVSLRHYYNWLTRQRY | 224 |
| 221 | APEKPEEDAPEEELQRYYVSLRHYYNWLTRQRY | 225 |
| 222 | APEKPEEDASAEELQQYYVSLRHYYNWLTRQRY | 226 |
| 223 | APEKPEEAAEPEELQRYYVSLRHYYNWLTRQRY | 227 |
| 224 | APEKPGEEASPEELEQYYVSLRHYYNWLTRQRY | 228 |
| 225 | APEKPGEDAEPEELEQYYVSLRHYYNWLTRQRY | 229 |
| 226 | APEKPEEAASPEETQRYYVSLRHYYNWLTRQRY | 230 |
| 227 | APEKAEEDASPEELEQYYVSLRHYYNWLTRQRY | 231 |
| 228 | APEKPEEDASPEELQQYYVSLRAYYNWLTRQRY | 232 |
| 229 | APEKPEEDASPEELQRYYVSARHYYNWLTRQRY | 233 |
| 230 | APEKPEEDASPEEIQRYYVALRHYYNWLTRQRY | 234 |
| 231 | APEKPEEDATPEELQKYYVSLRHYYNWLTRQRY | 235 |
| 232 | APAKPPEDASPEELQRYYVELRHYYNWLTRQRY | 236 |
| 233 | APEKPEEAASPEELQQYYVSLRHYYNWLTRQRY | 237 |
| 234 | APAKPEEEASPEELQRYYVSLRHYYNWLTRQRY | 238 |
| 235 | APEKPEEDASHypEELQRYYVSLRHYYNWLTRQRY | 239 |
| 236 | APEKPEEEAPAEELQRYYVSLRHYYNWLTRQRY | 240 |
| 237 | APEKPEEDASPEELQRYYVEVRHYYNWLTRQRY | 241 |
| 238 | APEKTEEEASPEELQRYYVSLRHYYNWLTRQRY | 242 |
| 239 | APEKPEEDASEEELQRYEVSLRHYYNWLTRQRY | 243 |
| 240 | APEKPEEDASPEALQRYYVSLRHYYNWLTRQRY | 244 |
| 241 | APEKPEEEASPEEIQRYYVSLRHYYNWLTRQRY | 245 |
| 242 | APEKPEADASAEELQRYYVSLRHYYNWLTRQRY | 246 |
| 243 | APEKPEEEASPEELQQYYVSLRHYYNWLTRQRY | 247 |
| 244 | APEKPEEDAEPAELQRYYVSLRHYYNWLTRQRY | 248 |

10. The PYY analogue according to claim 1, wherein the PYY analogue is in the form of pharmaceutically acceptable salt.

11. The PYY analogue according to claim 1, wherein the PYY analogue is in the form of a non-salt.

12. The PYY analogue according to claim 1, or a pharmaceutically acceptable salt thereof, wherein the binding affinity (Ki) towards hNPY2R is below 100 nM.

13. The PYY analogue according to claim 1, or a pharmaceutically acceptable salt thereof, wherein the solubility of the PYY analogue is greater than 1.0 mg/ml at pH 6.

14. A pharmaceutical composition comprising at least one PYY analogue according to claim 1, or a pharmaceutically acceptable salt thereof, and one or more pharmaceutically acceptable excipients.

15. A method for treating a condition or disease related or caused by excess body weight or excess body weight gain, the method comprising administering to a patient in need thereof a pharmaceutically effective amount of a PYY analogue according to claim 1, or a pharmaceutically acceptable salt thereof.

16. A method for treating obesity or an obesity-related condition, the method comprising administering to a patient in need thereof a PYY analogue according to claim 1, or a pharmaceutically acceptable salt thereof, wherein the obesity-related condition is selected from the group consisting of type 2 diabetes, hypertension, dyslipidemia, sleep apnea and cardiovascular disease.

17. A method for treating obesity and/or an obesity-related condition, the method comprising administering to a patient in need thereof a therapeutically effective amount of a PYY analogue according to claim 1, or a pharmaceutically acceptable salt thereof, wherein the obesity-related condition is selected from the group consisting of dyslipidemia, hepatic steatosis, NAFLD, NASH, kidney failure and atherosclerosis.

18. A method for treating diabetes, obesity, dyslipidemia or hypertension, the method comprising administering to a patient in need thereof a pharmaceutically effective amount of a PYY analogue according to claim 1, or a pharmaceutically acceptable salt thereof.

19. A method for treating diabetes, obesity, or an obesity-related condition, the method comprising administering to a patient in need thereof
a pharmaceutically effective amount of a PYY analogue according claim 1, or a pharmaceutically acceptable salt thereof, and
an agent for the treatment of obesity selected from the group consisting of GIP, a GLP-1 receptor agonist, exendin-4, an exendin-4 analogue, a glucagon-GLP-1 dual agonist, a GLP-1/GIP/glucagon triple agonist, or an amylin receptor agonist.

20. A compound selected from the group consisting of:
N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]-ethoxy}ethoxy) acetamido]-[4A,7K,9E,10A,13T,18Q,22V,28Y,30W,31L]hPYY(4-36)iVal-APEK(C18DA-gGlu-OEG1-OEG2-) (Compound 14),
N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]-ethoxy}ethoxy) acetamido]-[4A,6A,7K,9E,17I,18Q,22V,28Y,30W,31L]hPYY(4-36) (Compound 23),
N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]-ethoxy}ethoxy) acetamido]-[4A,7K,9E,10A,17I,18Q,22V,28Y,30W,31L]hPYY(4-36) (Compound 53),
N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]-ethoxy}ethoxy) acetamido]-[4A,7K,9E,14A,18Q,22V,23A,28Y,30W,31L]hPYY(4-36) (Compound 74),
N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]-ethoxy}ethoxy) acetamido]-[4A,7K,9E,10A,13T,18Q,22V,28Y,30W,31L]hPYY(4-36) (Compound 106)
N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]-ethoxy}ethoxy) acetamido]-[4A,6A,7K,9E,18Q,22V,23A,28Y,30W,31L]hPYY(4-36) (Compound 117),
N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]-ethoxy}ethoxy) acetamido]-[4A,7K,9E,10A,18Q,22V,23E,28Y,30W,31L]hPYY(4-36) (Compound 135),
N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]-ethoxy}ethoxy) acetamido]-[4A,7K,9E,10A,13T,18Q,22V,28Y,30W,31L]hPYY(4-36) (Compound 171)
N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]-ethoxy}ethoxy) acetamido]-[4A,7K,9E,17I,18Q,22V,23A,28Y,30W,31L]hPYY(4-36) (Compound 230), and
N{alpha-4}-(3-methylbutanoyl)-N{epsilon-7}-[2-(2-{2-[2-(2-{2-[(4S)-4-carboxy-4-(17-carboxyheptadecanamido)butanamido]ethoxy}ethoxy)acetamido]-ethoxy}ethoxy) acetamido]-[4A,7K,9E,10A,13T,18Q,22V,28Y,30W,31L]hPYY(4-36) (Compound 231)
or a pharmaceutically acceptable salt thereof.


SEQUENCE LISTING (259 sequences)

SEQ ID NO: 1 - protein, 36 aa, Homo sapiens
  REGION 1..36: note = hNPY
  SITE 36: note = C-term amidation
YPSKPDNPGEDAPAEDMARYYSALRHYINLITRQRY

SEQ ID NO: 2 - protein, 36 aa, Homo sapiens
  REGION 1..36: note = hPYY(1-36)
  SITE 36: note = C-term amidation
YPIKPEAPGEDASPEELNRYYASLRHYLNLVTRQRY

SEQ ID NO: 3 - protein, 36 aa, Homo sapiens
  REGION 1..36: note = hPP
  SITE 36: note = C-term amidation
APLEPVYPGDNATPEQMAQYAADLRRYINMLTRPRY

SEQ ID NO: 4 - protein, 34 aa, Homo sapiens
  REGION 1..34: note = hPYY(3-36)
IKPEAPGEDASPEELNRYYASLRHYLNLVTRQRY

SEQ ID NO: 5 - protein, 33 aa, synthetic construct
  REGION 1..33: note = Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDAQPEELQEYYVSLRHYYNWLTRQRY

SEQ ID NO: 6 - protein, 33 aa, synthetic construct
  REGION 1..33: note = Synthetic PPY analogue polypeptide
  SITE 1: note = N-term 3-methylbutanoyl
  SITE 2: note = (4R)-4-hydroxy-L-proline
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
AXEKPEEDASPEELQRYYVSLRHYYNWLTRQRY

SEQ ID NO: 7 - protein, 33 aa, synthetic construct
  REGION 1..33: note = Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEEASPAELQRYYVSLRHYYNWLTRQRY

SEQ ID NO: 8 - protein, 33 aa, synthetic construct
  REGION 1..33: note = Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEELQRYYVSLRHYYNWLQRQRY

SEQ ID NO: 9 - protein, 33 aa, synthetic construct
  REGION 1..33: note = Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEEVSPEELQRYYVSLRHYYNWLTRQRY

SEQ ID NO: 10 - protein, 33 aa, synthetic construct
  REGION 1..33: note = Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEEASPEELQRYYVSARHYYNWLTRQRY

SEQ ID NO: 11 - protein, 33 aa, synthetic construct
  REGION 1..33: note = Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEELQAYYVSLRHYYNWLTRQRY

SEQ ID NO: 12 - protein, 33 aa, synthetic construct
  REGION 1..33: note = Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASEEELQQYYVSLRHYYNWLTRQRY

SEQ ID NO: 13 - protein, 33 aa, synthetic construct
  REGION 1..33: note = Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEEIQRYYVELRHYYNWLTRQRY

SEQ ID NO: 14 - protein, 33 aa, synthetic construct
  REGION 1..33: note = Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEADASPEETQRYYVSLRHYYNWLTRQRY

SEQ ID NO: 15 - protein, 33 aa, synthetic construct
  REGION 1..33: note = Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEEIQQYYVSLRHYYNWLTRQRY

SEQ ID NO: 16 - protein, 33 aa, synthetic construct
  REGION 1..33: note = Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDAPGEELQRYYVSLRHYYNWLTRQRY

SEQ ID NO: 17 - protein, 33 aa, synthetic construct
  REGION 1..33: note = Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDAAPEELQQYYVSLRHYYNWLTRQRY

SEQ ID NO: 18 - protein, 33 aa, synthetic construct
  REGION 1..33: note = Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEADATPEELQRYYVSLRHYYNWLTRQRY

SEQ ID NO: 19 - protein, 33 aa, synthetic construct
  REGION 1..33: note = Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEEIQRYEVSLRHYYNWLTRQRY

SEQ ID NO: 20 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEETQRYYVTLRHYYNWLTRQRY

SEQ ID NO: 21 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPAELQRYYVSLRHYYNWLTRQRY

SEQ ID NO: 22 - protein, 33 aa, synthetic construct
  REGION 1..33: note = Description of Artificial Sequence: Synthetic PPY analogue polypeptide <
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDAEPEELQQYYVSLRHYYNWLTRQRY

SEQ ID NO: 23 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEADASPEELQQYYVSLRHYYNWLTRQRY

SEQ ID NO: 24 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDAAPEELQRYYVELRHYYNWLTRQRY

SEQ ID NO: 25 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEELQKYYVSLRHYYNWLTRQRY

SEQ ID NO: 26 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEELQRYYTSLRHYYNWLTRQRY

SEQ ID NO: 27 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APAKPEEDASPEEIQRYYVSLRHYYNWLTRQRY

SEQ ID NO: 28 - protein, 33 aa, synthetic construct
  REGION 1..33: note = Description of Artificial Sequence: Synthetic PPY analogue polypeptide <
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEELQRYYVSLRKYYNWLTRQRY

SEQ ID NO: 29 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEEASAEELQRYYVSLRHYYNWLTRQRY

SEQ ID NO: 30 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPPEDATPEELQRYYVSLRHYYNWLTRQRY

SEQ ID NO: 31 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPPEDAEPEELQRYYVSLRHYYNWLTRQRY

SEQ ID NO: 32 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDTSPEELQRYYVELRHYYNWLTRQRY

SEQ ID NO: 33 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEAPASPEELQRYYVELRHYYNWLTRQRY

SEQ ID NO: 34 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEADASEEELQRYYVSLRHYYNWLTRQRY

SEQ ID NO: 35 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEETQRYYVALRHYYNWLTRQRY

SEQ ID NO: 36 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDTSPEELQRYEVSLRHYYNWLTRQRY

SEQ ID NO: 37 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEPASPEELQRYYVELRHYYNWLTRQRY

SEQ ID NO: 38 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEELQRYYVSTRHYYNWLTRQRY

SEQ ID NO: 39 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDAAPEETQRYYVSLRHYYNWLTRQRY

SEQ ID NO: 40 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKAEEDAEPEELQRYYVSLRHYYNWLTRQRY

SEQ ID NO: 41 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEETQRYEVSLRHYYNWLTRQRY

SEQ ID NO: 42 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPPEDASPEELQRYYVALRHYYNWLTRQRY

SEQ ID NO: 43 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPPADAEPEELQRYYVSLRHYYNWLTRQRY

SEQ ID NO: 44 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDATPEETQRYYVSLRHYYNWLTRQRY

SEQ ID NO: 45 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEEATPEELQRYYVSLRHYYNWLTRQRY

SEQ ID NO: 46 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEAASPEELQRYYVSTRHYYNWLTRQRY

SEQ ID NO: 47 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEAPASPEELQRYYVSLRHYYNWLTRQRY

SEQ ID NO: 48 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEEASPEELQRYYVSLRHYYNWITRQRY

SEQ ID NO: 49 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEEASPESLQRYYVSLRHYYNWLTRQRY

SEQ ID NO: 50 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEEASPEELQRYYVSLRHYYNWLTRQRY

SEQ ID NO: 51 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPPADASPEELQRYYVSLRHYYNWLTRQRY

SEQ ID NO: 52 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDTEPEELQRYYVSLRHYYNWLTRQRY

SEQ ID NO: 53 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEELQQYYVELRHYYNWLTRQRY

SEQ ID NO: 54 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASAEELQRYYVSLRHYYNWITRQRY

SEQ ID NO: 55 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKAEEEASPEELQRYYVSLRHYYNWLTRQRY

SEQ ID NO: 56 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKAEEDASEEELQRYYVSLRHYYNWLTRQRY

SEQ ID NO: 57 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEADASPEEIQRYYVSLRHYYNWLTRQRY

SEQ ID NO: 58 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEELQRYYVSLRHYYNWLTRQRY

SEQ ID NO: 59 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEEAQRYYVSLRHYYNWLTRQRY

SEQ ID NO: 60 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEADAEPEELQRYYVSLRHYYNWLTRQRY

SEQ ID NO: 61 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASAEEIQRYYVSLRHYYNWLTRQRY

SEQ ID NO: 62 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEELQQYYVSLRHYYNWLTRQRY

SEQ ID NO: 63 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEETEQYYVSLRHYYNWLTRQRY

SEQ ID NO: 64 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEAASPEELEQYYVSLRHYYNWLTRQRY

SEQ ID NO: 65 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPPEDASPEEIQRYYVSLRHYYNWLTRQRY

SEQ ID NO: 66 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPGEDASPEELERYYVSLRHYYNWLTRQRY

SEQ ID NO: 67 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEELQRYYVSLRAYYNWLTRQRY

SEQ ID NO: 68 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPGEDASPEELQRYYVSLRHYYNWLTRQRY

SEQ ID NO: 69 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEELQRYYVEIRHYYNWLTRQRY

SEQ ID NO: 70 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASAEETQRYYVSLRHYYNWLTRQRY

SEQ ID NO: 71 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDSAPEELQRYYVSLRHYYNWLTRQRY

SEQ ID NO: 72 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEEASPEELQAYYVSLRHYYNWLTRQRY

SEQ ID NO: 73 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDSSAEELQRYYVSLRHYYNWLTRQRY

SEQ ID NO: 74 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEAASPEELQRYEVSLRHYYNWLTRQRY

SEQ ID NO: 75 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEEASPSELQRYYVSLRHYYNWLTRQRY

SEQ ID NO: 76 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEEGSPEELQRYYVSLRHYYNWLTRQRY

SEQ ID NO: 77 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEEASPEELQRYYVSLRAYYNWLTRQRY

SEQ ID NO: 78 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASAEELQRYYVALRHYYNWLTRQRY

SEQ ID NO: 79 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASAEELQRYYVTLRHYYNWLTRQRY

SEQ ID NO: 80 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPGEDASPEELQRYYVSLRHYYNALTRQRY

SEQ ID NO: 81 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASEEELQRYYVELRHYYNWLTRQRY

SEQ ID NO: 82 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDAEPEELQQYYVTLRHYYNWLTRQRY

SEQ ID NO: 83 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEEASEEELQRYYVSLRHYYNWLTRQRY

SEQ ID NO: 84 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEEASPEETQRYYVSLRHYYNWLTRQRY

SEQ ID NO: 85 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEELQRYYVSIRHYYNWLTRQRY

SEQ ID NO: 86 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEALQRYYVALRHYYNWLTRQRY

SEQ ID NO: 87 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEADASPAELQRYYVSLRHYYNWLTRQRY

SEQ ID NO: 88 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEEIQRYYVSLRHYYNWLTRQRY

SEQ ID NO: 89 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEELQRYAVSLRHYYNWLTRQRY

SEQ ID NO: 90 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEADASPEELQRYEVSLRHYYNWLTRQRY

SEQ ID NO: 91 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDSSPAELQRYYVSLRHYYNWLTRQRY

SEQ ID NO: 92 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPPEDASPEELQRYYVELRHYYNWLTRQRY

SEQ ID NO: 93 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEEAEPEELQRYYVSLRHYYNWLTRQRY

SEQ ID NO: 94 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEELQRYEVSLRHYYNWLTRQRY

SEQ ID NO: 95 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDAPAEELQRYYVELRHYYNWLTRQRY

SEQ ID NO: 96 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDTSPEELQRYYVSLRHYYNWLTRQRY

SEQ ID NO: 97 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASAEELQKYYVSLRHYYNWLTRQRY

SEQ ID NO: 98 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEETQRYYVELRHYYNWLTRQRY

SEQ ID NO: 99 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPAEIQRYYVSLRHYYNWLTRQRY

SEQ ID NO: 100 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEADASPEELQRYYVSLRHYYNWLTRQRY

SEQ ID NO: 101 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEELQRYYVALRHYYNWLTRQRY

SEQ ID NO: 102 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEELQRYYSALRHYYNWLTRQRY

SEQ ID NO: 103 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDAEAEELQRYYVSLRHYYNWLTRQRY

SEQ ID NO: 104 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APAKPPEDASPEELQRYYVSLRHYYNWLTRQRY

SEQ ID NO: 105 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDATPEELQRYYVELRHYYNWLTRQRY

SEQ ID NO: 106 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEELQRYYVTLRHYYNWLTRQRY

SEQ ID NO: 107 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APAKPEEDASEEELQRYYVSLRHYYNWLTRQRY

SEQ ID NO: 108 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASAEELQRYYVSLRHYYNWLTRQRY

SEQ ID NO: 109 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDAEPEELQEYYVSLRHYYNWLTRQRY

SEQ ID NO: 110 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APAKPEEDATPEELQRYYVSLRHYYNWLTRQRY

SEQ ID NO: 111 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEELQKYYVALRHYYNWLTRQRY

SEQ ID NO: 112 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEELQRYYVELRHYYNWLTRQRY

SEQ ID NO: 113 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEAASPEELQRYYVSLRHYYNWLTRQRY

SEQ ID NO: 114 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDAEPEEIQRYYVSLRHYYNWLTRQRY

SEQ ID NO: 115 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDAEPEELQRYYVELRHYYNWLTRQRY

SEQ ID NO: 116 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDATPEELQRYYVSLRHYYNWLTRQRY

SEQ ID NO: 117 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKAEEDATPEELQRYYVSLRHYYNWLTRQRY

SEQ ID NO: 118 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEELQRYYVSLRHYHWYLTRQRY

SEQ ID NO: 119 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEEASPEALQRYYVSLRHYYNWLTRQRY

SEQ ID NO: 120 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDAAAEELQRYYVSLRHYYNWLTRQRY

SEQ ID NO: 121 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APAKPEEDASPEELQRYYVALRHYYNWLTRQRY

SEQ ID NO: 122 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASAAELQRYYVSLRHYYNWLTRQRY

SEQ ID NO: 123 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEEASPEELQRYYVTLRHYYNWLTRQRY

SEQ ID NO: 124 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPGEDAEEEELQEYYVSLRHYYNWLTRQRY

SEQ ID NO: 125 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APAKPEADASPEELQRYYVSLRHYYNWLTRQRY

SEQ ID NO: 126 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDAAPEELQRYYVSLRHYYNWLTRQRY

SEQ ID NO: 127 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEELQRYYVSLRHYWYHLTRQRY

SEQ ID NO: 128 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDAAPEEIQRYYVSLRHYYNWLTRQRY

SEQ ID NO: 129 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPGEDASPEELQRYYVSLRHYYNWATRQRY

SEQ ID NO: 130 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APAKPEEDSSPEELQRYYVSLRHYYNWLTRQRY

SEQ ID NO: 131 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APAKPEEDASAEELQRYYVSLRHYYNWLTRQRY

SEQ ID NO: 132 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDAPAEEIQRYYVSLRHYYNWLTRQRY

SEQ ID NO: 133 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEETQRYYTALRHYYNWLTRQRY

SEQ ID NO: 134 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDTSAEELQRYYVSLRHYYNWLTRQRY

SEQ ID NO: 135 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKAEEDASPEELQRYYVELRHYYNWLTRQRY

SEQ ID NO: 136 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEELQKYYVTLRHYYNWLTRQRY

SEQ ID NO: 137 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEEVQRYYVELRHYYNWLTRQRY

SEQ ID NO: 138 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEEIQRYYTELRHYYNWLTRQRY

SEQ ID NO: 139 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEADASPEELQRYYVELRHYYNWLTRQRY

SEQ ID NO: 140 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEADSSPEELQRYYVSLRHYYNWLTRQRY

SEQ ID NO: 141 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEELQRYYVSLRHYYNWITRQRY

SEQ ID NO: 142 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APAKPEEDASPEELQQYYVSLRHYYNWLTRQRY

SEQ ID NO: 143 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEESSPEELQRYYVSLRHYYNWLTRQRY

SEQ ID NO: 144 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPGEDASPEELEQYYVSLRHYYNWLTRQRY

SEQ ID NO: 145 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEAASPEELQRYYVSARHYYNWLTRQRY

SEQ ID NO: 146 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPPEEASPEELQRYYVSLRHYYNWLTRQRY

SEQ ID NO: 147 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDATPEELQRYYVALRHYYNWLTRQRY

SEQ ID NO: 148 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKAEEDASPEELQQYYVSLRHYYNWLTRQRY

SEQ ID NO: 149 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDSSPEEIQRYYVSLRHYYNWLTRQRY

SEQ ID NO: 150 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEEASPEELQRYYVSTRHYYNWLTRQRY

SEQ ID NO: 151 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEETQKYYVSLRHYYNWLTRQRY

SEQ ID NO: 152 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDAPAEELQRYYVSLRHYYNWLTRQRY

SEQ ID NO: 153 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEAASPEELQRYYVELRHYYNWLTRQRY

SEQ ID NO: 154 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDAEPEELQKYYVSLRHYYNWLTRQRY

SEQ ID NO: 155 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEAEASPEELQRYYVSLRHYYNWLTRQRY

SEQ ID NO: 156 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKAEEDASPEEIQRYYVSLRHYYNWLTRQRY

SEQ ID NO: 157 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKVEEEASPEELQRYYVSLRHYYNWLTRQRY

SEQ ID NO: 158 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEEASPEELQRYEVSLRHYYNWLTRQRY

SEQ ID NO: 159 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEETQRYYVSLRHYYNWLTRQRY

SEQ ID NO: 160 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASEEELEQYYVSLRHYYNWLTRQRY

SEQ ID NO: 161 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APAKPEEDASPEETQRYYVSLRHYYNWLTRQRY

SEQ ID NO: 162 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEELQRYYVSLRHYWNYLTRQRY

SEQ ID NO: 163 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEADASPEELEQYYVSLRHYYNWLTRQRY

SEQ ID NO: 164 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEADAEPEELQRYYTSLRHYYNWLTRQRY

SEQ ID NO: 165 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDATPEELQQYYVSLRHYYNWLTRQRY

SEQ ID NO: 166 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEELQRYEVSLRAYYNWLTRQRY

SEQ ID NO: 167 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEAASPEELQRYYVALRHYYNWLTRQRY

SEQ ID NO: 168 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASEEELQEYYVSLRHYYNWLTRQRY

SEQ ID NO: 169 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEELQRYQVSLRHYYNWLTRQRY

SEQ ID NO: 170 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPPADASPEELQRYYVELRHYYNWLTRQRY

SEQ ID NO: 171 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEELQQYEVSLRHYYNWLTRQRY

SEQ ID NO: 172 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDAEPEELQRYYVSLRHYYNWITRQRY

SEQ ID NO: 173 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEEASPEELQRYYVALRHYYNWLTRQRY

SEQ ID NO: 174 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEEASPEELEQYYVSLRHYYNWLTRQRY

SEQ ID NO: 175 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDATPEEIQRYYVSLRHYYNWLTRQRY

SEQ ID NO: 176 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDAEPEELQRYYVALRHYYNWLTRQRY

SEQ ID NO: 177 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APAKPEEDASPEELQRYYVELRHYYNWLTRQRY

SEQ ID NO: 178 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APAKPEEDAEPEELQRYYVSLRHYYNWLTRQRY

SEQ ID NO: 179 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDAAPEELQRYYVALRHYYNWLTRQRY

SEQ ID NO: 180 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASEEELQRYYVSLRHYYNWLTRQRY

SEQ ID NO: 181 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDSSPEELQRYYVELRHYYNWLTRQRY

SEQ ID NO: 182 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDATPEETERYYVSLRHYYNWLTRQRY

SEQ ID NO: 183 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APAKPEEDASPAELQRYYVSLRHYYNWLTRQRY

SEQ ID NO: 184 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEETQQYYVSLRHYYNWLTRQRY

SEQ ID NO: 185 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEADASPEELQRYYVALRHYYNWLTRQRY

SEQ ID NO: 186 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDGSPEELQRYYVELRHYYNWLTRQRY

SEQ ID NO: 187 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPPEDASPEELQRYYVSLRHYYNWLTRQRY

SEQ ID NO: 188 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEAASPEELQRYYVTLRHYYNWLTRQRY

SEQ ID NO: 189 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEEASPEELQRYYVELRHYYNWLTRQRY

SEQ ID NO: 190 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASAEELQRYYVELRHYYNWLTRQRY

SEQ ID NO: 191 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEELQRYYVSLRHQYNWLTRQRY

SEQ ID NO: 192 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDAEPEELQRYYVSLRHYYNWLTRQRY

SEQ ID NO: 193 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASEAELQRYYVSLRHYYNWLTRQRY

SEQ ID NO: 194 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEEAPPEELQRYYVSLRHYYNWLTRQRY

SEQ ID NO: 195 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEELQRYYISLRHYYNWLTRQRY

SEQ ID NO: 196 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDAAPAELQRYYVSLRHYYNWLTRQRY

SEQ ID NO: 197 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASEEEIQRYYVSLRHYYNWLTRQRY

SEQ ID NO: 198 - protein, 33 aa, synthetic construct
  REGION 1..33: note = Description of Artificial Sequence: Synthetic PPY analogue polypeptide
  SITE 1: note = N-term 3-methylbutanoyl
  SITE 2: note = (4R)-4-hydroxy-L-proline
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
AXEKPEEDASPEELQRYYVSLRHYYNWTLRQRY

SEQ ID NO: 199 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEALQRYYVELRHYYNWLTRQRY

SEQ ID NO: 200 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APAKPPEDAEPEELQRYYVSLRHYYNWLTRQRY

SEQ ID NO: 201 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APAKPEADASPEELQRYYVELRHYYNWLTRQRY

SEQ ID NO: 202 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDAEPEALQRYYVSLRHYYNWLTRQRY

SEQ ID NO: 203 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDAEPEETQRYYVSLRHYYNWLTRQRY

SEQ ID NO: 204 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 5: note = (4R)-4-hydroxy-L-proline
  SITE 33: note = C-term amidation
APEKXEEDASPEELQRYYVSLRHYYNWLTRQRY

SEQ ID NO: 205 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPQEDAEPEELQEYYVSLRHYYNWLTRQRY

SEQ ID NO: 206 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDAAPEELQKYYVSLRHYYNWLTRQRY

SEQ ID NO: 207 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APAKPEEDASPEELQRYYVSLRHYYNWLTRQRY

SEQ ID NO: 208 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEEASPEEVQRYYVSLRHYYNWLTRQRY

SEQ ID NO: 209 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEEASPEELQKYYVSLRHYYNWLTRQRY

SEQ ID NO: 210 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEELEQYEVSLRHYYNWLTRQRY

SEQ ID NO: 211 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APAKPEADAEPEELQRYYVSLRHYYNWLTRQRY

SEQ ID NO: 212 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASEEETQRYYVSLRHYYNWLTRQRY

SEQ ID NO: 213 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDSEPEELQRYYVSLRHYYNWLTRQRY

SEQ ID NO: 214 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKIEEEASPEELQRYYVSLRHYYNWLTRQRY

SEQ ID NO: 215 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDATAEELQRYYVSLRHYYNWLTRQRY

SEQ ID NO: 216 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDTAPEELQRYYVSLRHYYNWLTRQRY

SEQ ID NO: 217 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEETSPEELQRYYVSLRHYYNWLTRQRY

SEQ ID NO: 218 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASEEELQRYYVALRHYYNWLTRQRY

SEQ ID NO: 219 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPAELQRYYVELRHYYNWLTRQRY

SEQ ID NO: 220 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APAKPEEDASPEELEQYYVSLRHYYNWLTRQRY

SEQ ID NO: 221 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPAELQRYYVALRHYYNWLTRQRY

SEQ ID NO: 222 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDSSPEELQRYYVALRHYYNWLTRQRY

SEQ ID NO: 223 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEEAAPEELQRYYVSLRHYYNWLTRQRY

SEQ ID NO: 224 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEAASAEELQRYYVSLRHYYNWLTRQRY

SEQ ID NO: 225 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDAPEEELQRYYVSLRHYYNWLTRQRY

SEQ ID NO: 226 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASAEELQQYYVSLRHYYNWLTRQRY

SEQ ID NO: 227 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEAAEPEELQRYYVSLRHYYNWLTRQRY

SEQ ID NO: 228 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPGEEASPEELEQYYVSLRHYYNWLTRQRY

SEQ ID NO: 229 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPGEDAEPEELEQYYVSLRHYYNWLTRQRY

SEQ ID NO: 230 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEAASPEETQRYYVSLRHYYNWLTRQRY

SEQ ID NO: 231 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKAEEDASPEELEQYYVSLRHYYNWLTRQRY

SEQ ID NO: 232 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEELQQYYVSLRAYYNWLTRQRY

SEQ ID NO: 233 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEELQRYYVSARHYYNWLTRQRY

SEQ ID NO: 234 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEEIQRYYVALRHYYNWLTRQRY

SEQ ID NO: 235 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDATPEELQKYYVSLRHYYNWLTRQRY

SEQ ID NO: 236 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APAKPPEDASPEELQRYYVELRHYYNWLTRQRY

SEQ ID NO: 237 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEAASPEELQQYYVSLRHYYNWLTRQRY

SEQ ID NO: 238 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APAKPEEEASPEELQRYYVSLRHYYNWLTRQRY

SEQ ID NO: 239 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 11: note = (4R)-4-hydroxy-L-proline
  SITE 33: note = C-term amidation
APEKPEEDASXEELQRYYVSLRHYYNWLTRQRY

SEQ ID NO: 240 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEEAPAEELQRYYVSLRHYYNWLTRQRY

SEQ ID NO: 241 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEELQRYYVEVRHYYNWLTRQRY

SEQ ID NO: 242 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKTEEEASPEELQRYYVSLRHYYNWLTRQRY

SEQ ID NO: 243 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASEEELQRYEVSLRHYYNWLTRQRY

SEQ ID NO: 244 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDASPEALQRYYVSLRHYYNWLTRQRY

SEQ ID NO: 245 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEEASPEEIQRYYVSLRHYYNWLTRQRY

SEQ ID NO: 246 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEADASAEELQRYYVSLRHYYNWLTRQRY

SEQ ID NO: 247 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEEASPEELQQYYVSLRHYYNWLTRQRY

SEQ ID NO: 248 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
APEKPEEDAEPAELQRYYVSLRHYYNWLTRQRY

SEQ ID NO: 249 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
RPEKPGEDASPEELQRYYVSLRHYYNWLTRQRY

SEQ ID NO: 250 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
RPEKPGEDASPEELQRYYVSLAHYYNWLTRQRY

SEQ ID NO: 251 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
RPEKPGEDASPEELQRYYVSLRAYYNWLTRQRY

SEQ ID NO: 252 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
RPEKPGEDASPEELQRYYVSLRHYYNWLTAQRY

SEQ ID NO: 253 - protein, 33 aa, synthetic construct
  REGION 1..33: note = Description of Artificial Sequence: Synthetic PPY analogue polypeptide
  SITE 1: note = N-term 3-methylbutanoyl
  SITE 4: note = The epsilon amino group of Lys has the following substituent: [2- (2-{2-[2-(2-{2-[(4S)-4-carboxy-4-(17-carb oxyheptadecanamido)butan amido]ethoxy}ethoxy)acetamido]ethoxy}ethoxy)acetyl
  SITE 33: note = C-term amidation
RPEKPGEDASPEELQRYYVSLRHYYNWLTRQAY

SEQ ID NO: 254 - protein, 33 aa, synthetic construct
  REGION 1..33: note = Amino acid sequence of formula I
  REGION 1..33: note = Description of Artificial Sequence: Synthetic polypeptide
  VARIANT 2: note = Pro or 4Hyp
  VARIANT 3: note = Ala or Glu
  VARIANT 5: note = Ala, Ile, Pro, Thr, Val or 4Hyp
  VARIANT 6: note = Glu, Gly, Gln or Pro
  VARIANT 7: note = Ala or Glu
  VARIANT 8: note = Ala, Asp, Glu or Pro
  VARIANT 9: note = Ala, Gly, Ser, Thr or Val
  VARIANT 10: note = Ala, Glu, Ser, Gln, Thr or Pro
  VARIANT 11: note = Ala, Glu, Gly, Pro or 4Hyp
  VARIANT 12: note = Ala, Glu or Ser
  VARIANT 13: note = Ala, Glu or Ser
  VARIANT 14: note = Ala, Ile, Leu, Thr or Val
  VARIANT 15: note = Glu or Gln
  VARIANT 16: note = Ala, Glu, Arg, Lys or Gln
  VARIANT 18: note = Ala, Glu, Gln or Tyr
  VARIANT 19: note = Ile, Ser, Thr or Val
  VARIANT 20: note = Ala, Glu, Ser or Thr
  VARIANT 21: note = Ala, Ile, Leu, Thr or Val
  VARIANT 23: note = Ala, His or Lys
  VARIANT 24: note = Gln or Tyr
  VARIANT 25: note = His, Trp or Tyr
  VARIANT 26: note = Asn, Trp or Tyr
  VARIANT 27: note = Ala, His, Trp or Tyr
  VARIANT 28: note = Ala, Ile, Leu or Thr
  VARIANT 29: note = Gln, Leu or Thr
AXXKXXXXXXXXXXXXYXXXXRXXXXXXXRQRY

SEQ ID NO: 255 - protein, 33 aa, synthetic construct
  REGION 1..33: note = Description of Artificial Sequence: Synthetic polypeptide
  REGION 1..33: note = Amino acid sequence of formula II
  VARIANT 2: note = Pro or 4Hyp
  VARIANT 3: note = Ala or Glu
  VARIANT 5: note = Ala, Pro or 4Hyp
  VARIANT 6: note = Gln, Gly, Glu or Pro
  VARIANT 7: note = Ala or Glu
  VARIANT 8: note = Ala, Asp, Glu or Pro
  VARIANT 9: note = Ala, Gly or Ser
  VARIANT 10: note = Ala, Glu, Ser, Thr or Pro
  VARIANT 11: note = Ala, Glu, Pro or 4Hyp
  VARIANT 12: note = Ala, Glu or Ser
  VARIANT 13: note = Ala, Glu or Ser
  VARIANT 14: note = Ile, Leu, Thr or Val
  VARIANT 15: note = Gln or Glu
  VARIANT 16: note = Ala, Glu, Arg, Lys or Gln
  VARIANT 18: note = Ala, Glu, Gln or Tyr
  VARIANT 19: note = Ile, Ser, Thr or Val
  VARIANT 20: note = Ala, Glu, Ser or Thr
  VARIANT 21: note = Ala, Ile, Leu, Thr or Val
  VARIANT 23: note = Ala, His or Lys
  VARIANT 25: note = Trp or Tyr
  VARIANT 26: note = Asn or Tyr
  VARIANT 27: note = His, Trp or Tyr
  VARIANT 28: note = Ala, Ile or Leu
  VARIANT 29: note = Gln or Thr
AXXKXXXXXXXXXXXXYXXXXRXYXXXXXRQRY

SEQ ID NO: 256 - protein, 33 aa, synthetic construct
  REGION 1..33: note = Description of Artificial Sequence: Synthetic polypeptide
  REGION 1..33: note = Amino acid sequence of formula III
  VARIANT 3: note = Ala or Glu
  VARIANT 6: note = Glu or Gly
  VARIANT 7: note = Ala or Glu
  VARIANT 8: note = Ala, Asp, Glu or Pro
  VARIANT 9: note = Ala or Ser
  VARIANT 10: note = Ala, Glu, Ser, Thr or Pro
  VARIANT 11: note = Ala, Glu or Pro
  VARIANT 12: note = Ala or Glu
  VARIANT 13: note = Ala or Glu
  VARIANT 14: note = Ile, Leu, Thr or Val
  VARIANT 15: note = Glu or Gln
  VARIANT 16: note = Ala, Glu, Arg, Lys or Gln
  VARIANT 18: note = Glu or Tyr
  VARIANT 20: note = Ala, Glu, Ser or Thr
APXKPXXXXXXXXXXXYXVXLRHYYNWLTRQRY

SEQ ID NO: 257 - protein, 33 aa, synthetic construct
  REGION 1..33: note = Description of Artificial Sequence: Synthetic polypeptide
  REGION 1..33: note = Amino acid sequence of formula IV
  VARIANT 3: note = Ala or Glu
  VARIANT 7: note = Ala or Glu
  VARIANT 8: note = Ala, Asp or Glu
  VARIANT 10: note = Ala, Glu, Ser or Thr
  VARIANT 11: note = Ala, Pro or 4Hyp
  VARIANT 14: note = Ile or Leu
  VARIANT 16: note = Arg, Lys or Gln
  VARIANT 20: note = Ala, Glu or Ser
APXKPEXXAXXEEXQXYYVXLRHYYNWLTRQRY

SEQ ID NO: 258 - protein, 4 aa, synthetic construct
  REGION 1..4: note = Description of Artificial Sequence: Synthetic peptide
RQRY

SEQ ID NO: 259 - protein, 7 aa, synthetic construct
  REGION 1..7: note = Description of Artificial Sequence: Synthetic peptide
WLTRQRY